(19) United States
(12) Patent Application Publication
(10) Pub. No.: US 2025/0230150 A1
(43) Pub. Date: Jul. 17, 2025

(54) PROCESS AND INTERMEDIATES USEFUL FOR PREPARING NIRMATRELVIR

(71) Applicant: Pfizer Inc., New York, NY (US)

(72) Inventors: Christophe Philippe Allais, Ledyard, CT (US); Nga My Do, Groton, CT (US); Samir Ashok Kulkarni, East Lyme, CT (US); David William Place, East Greenwich, RI (US); John Anthony Ragan, Mystic, CT (US); Emma Leigh Rincon, Essex, CT (US); Rodney Matthew Weekly, Norwich, CT (US)

(73) Assignee: Pfizer Inc., New York, NY (US)

(21) Appl. No.: 18/876,789

(22) PCT Filed: Jun. 27, 2023
(86) PCT No.: PCT/IB2023/056621

§ 371 Date: Dec. 19, 2024

Related U.S. Application Data
(60) Provisional application No. 63/367,365, filed on Jun. 30, 2022.

(51) Int. Cl. C07D 413/12

(52) U.S. Cl. CPC C07D 413/12

(57) ABSTRACT

The present invention is directed to intermediates and an efficient process for preparing nirmatrelvir (compound of Formula I) and intermediates useful in the preparation of nirmatrelvir.

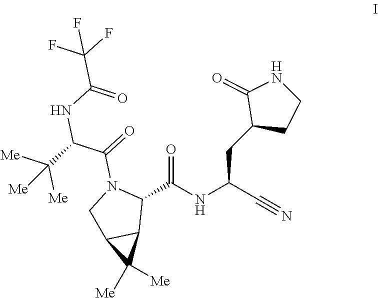 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C]

The present invention is directed to intermediates and an efficient process for preparing nirmatrelvir and intermediates useful in the preparation of nirmatrelvir. Nirmatrelvir is an antiviral compound with potent inhibitory activity against coronavirus 3CL proteases and is an active ingredient in the product Paxlovid® which has been authorized for use in the treatment of COVID-19. Nirmatrelvir and processes for its preparation have been disclosed in PCT International Patent Application WO 2021/250648 and US Patent Application Publication 2022/0062232 A1 and U.S. Pat. No. 11,351,149.

### SUMMARY OF THE INVENTION

The present invention provides intermediates and synthetic processes for preparing intermediates used in the preparation of nirmatrelvir which is depicted in Reaction Scheme 1, and which contains several process modifications compared to the previously disclosed processes. The product of Step 1 in Reaction Scheme 1 is sodium (1R,2S,5S)-6,6-dimethyl-3-azabicyclo[3.1.0]hexane-2-carboxylate which is a new salt form (sodium replacing lithium) compared to the prior process, and the Step 2 and Step 3 reaction conditions are modified. Additional solid form characterization data for several starting materials and intermediates used in the process are provided. A process for preparation and isolation of (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide, methyl ethyl ketone (MEK) solvate is presented along with characterization data for that compound.

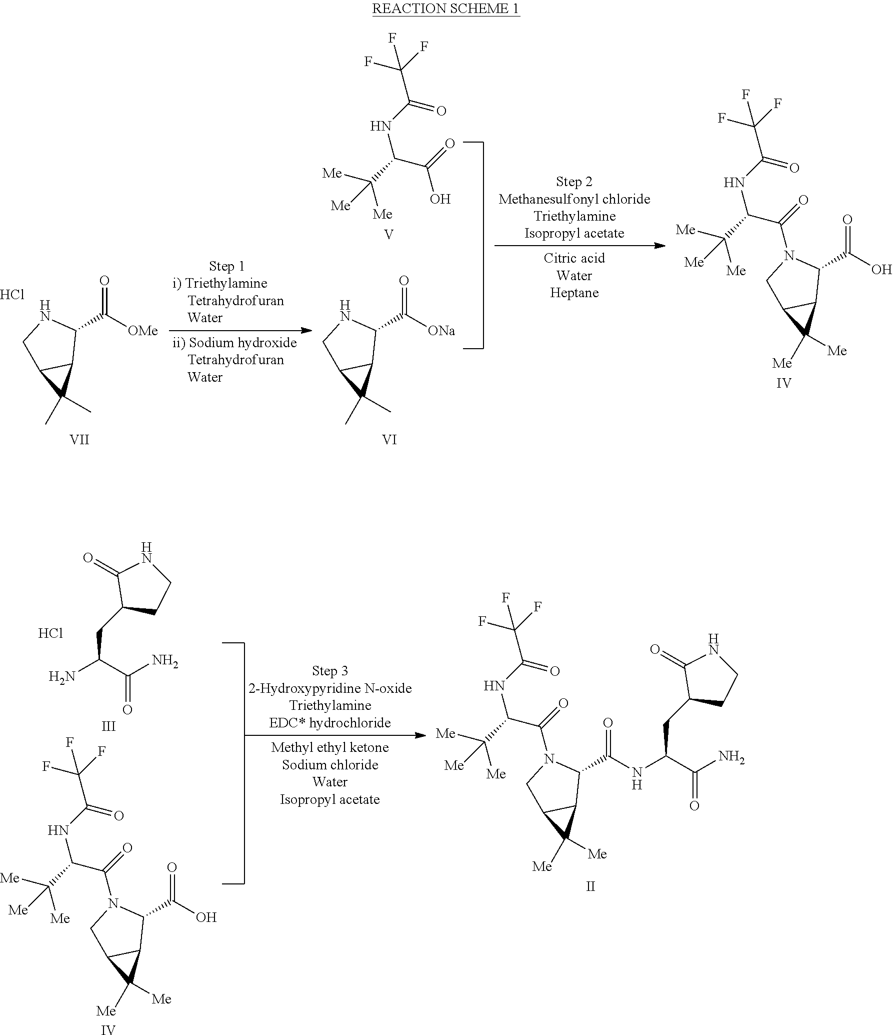

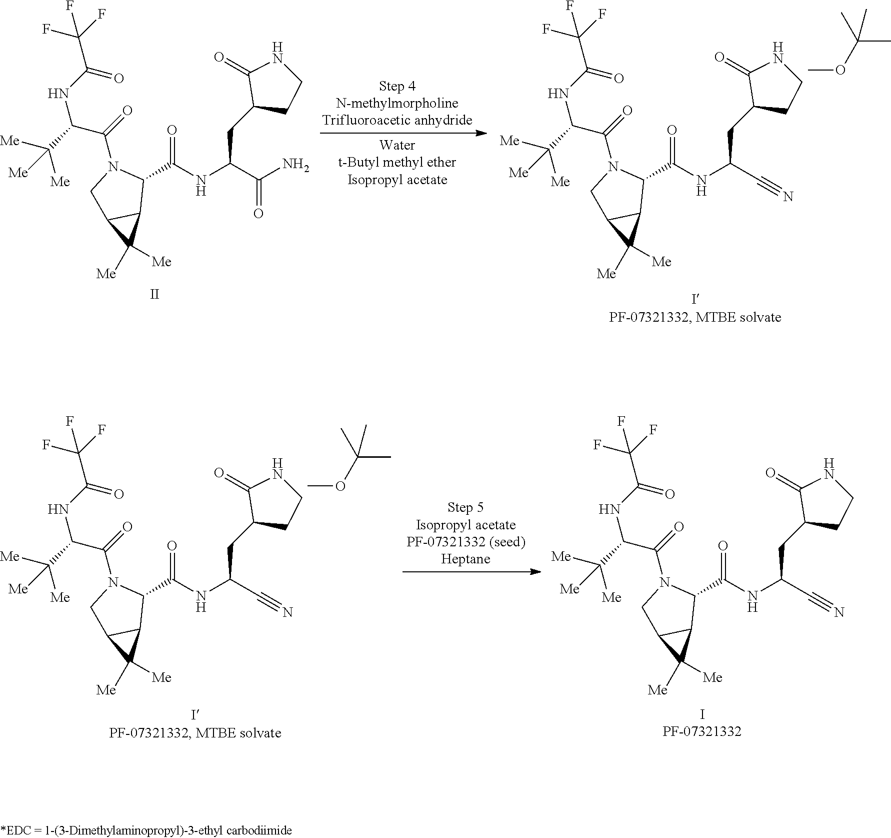

### BRIEF DESCRIPTION OF THE DRAWINGS

FIG. 1: Relationship between the initial concentration of API (PF-07321332, MTBE solvate) in isopropyl acetate (mL/g), the heptane addition time (in hours), the seed load (% wt PF-07321332, Form 1/wt of PF-07321332 MTBE solvate) and the seed size (in microns) on the final D[v, 0.5] counts in micron of particle size distributions (PSD's) of PF-07321332, Form 1.

FIG. 2: Correlation between seed size and final particle size distribution after crystallization of PF-07321332, Form 1 with 0.75 wt % seed load (blue circles) and 0.5 wt % seed load (orange circles) on laboratory scale (100 mL) experiments.

FIG. 3: Particle size distributions (PSDs) obtained for PF-07321332, Form 1 showing the D[v,0.5] in microns of more than 50 batches at one location.

FIG. 4: Particle size distributions (PSDs) obtained for PF-07321332, Form 1 showing the D[v,0.5] in microns of more than 50 batches at one location.

FIG. 5: PXRD pattern for(S)-3,3-dimethyl-2-(2,2,2-trifluoroacetamido)butanoic acid, compound V

FIG. 6: PXRD pattern for methyl (1R,2S,5S)-6,6-dimethyl-3-azabicyclo[3.1.0]hexane-2-carboxylate, hydrochloride salt; compound VII

FIG. 7: PXRD pattern of sodium (1R,2S,5S)-6,6-dimethyl-3-azabicyclo[3.1.0]hexane-2-carboxylate, compound VI, initial form

FIG. 8: PXRD pattern of sodium (1R,2S,5S)-6,6-dimethyl-3-azabicyclo[3.1.0]hexane-2-carboxylate, Form B

FIG. 9: PXRD pattern of sodium (1R,2S,5S)-6,6-dimethyl-3-azabicyclo[3.1.0]hexane-2-carboxylate, Material A

FIG. 10: PXRD pattern of (1R,2S,5S)-3-((S)-3,3-dimethyl-2-(2,2,2-trifluoroacetamido) butanoyl)-6,6-dimethyl-3-azabicyclo[3.1.0]hexane-2-carboxylic acid, Form 1

FIG. 11: PXRD pattern of (1R,2S,5S)-3-((S)-3,3-dimethyl-2-(2,2,2-trifluoroacetamido) butanoyl)-6,6-dimethyl-3-azabicyclo[3.1.0]hexane-2-carboxylic acid, New Form

FIG. 12: PXRD pattern for(S)-2-amino-3-((S)-2-oxopyrrolidin-3-yl)propanamide hydrochloride, Form 1

FIG. 13: PXRD pattern for (S)-2-amino-3-((S)-2-oxopyrrolidin-3-yl)propanamide hydrochloride, Form 2

FIG. 14: PXRD pattern for (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide, methyl ethyl ketone solvate

FIG. 15: PXRD pattern for PF-07321332 isopropyl acetate solvate

### DETAILED DESCRIPTION OF THE INVENTION

The following embodiments, EMB-1 to EMB-32 are representative embodiments of the present invention which should be construed in a non-limiting manner.

EMB-1 is the compound (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo [3.1.0]hexane-2-carboxamide, methyl ethyl ketone solvate.

EMB-2 is a process for preparing (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide, compound II

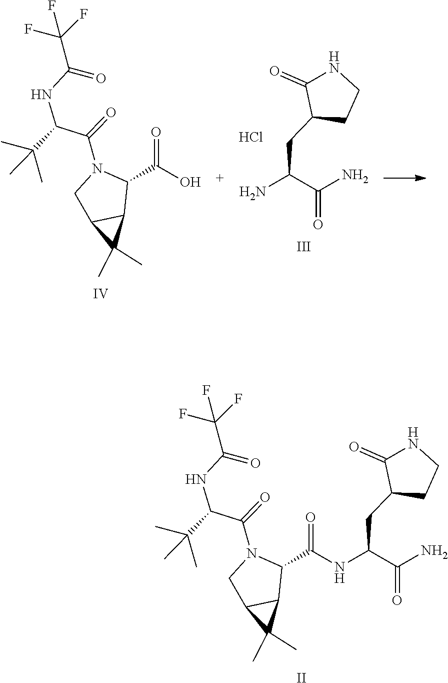

the process of reacting the compound of formula IV with the compound of formula III comprising the steps (a)-(d):
(a) (1R,2S,5S)-3-((S)-3,3-dimethyl-2-(2,2,2-trifluoroacetamido) butanoyl)-6,6-dimethyl-3-azabicyclo [3.1.0]hexane-2-carboxylic acid, compound IV and methyl ethyl ketone are combined followed by addition of 2-Hydroxypyridine N-oxide and triethylamine to provide a first mixture, S3-M1;
(b) (S)-2-amino-3-((S)-2-oxopyrrolidin-3-yl)propanamide hydrochloride, compound III, 1-(3-dimethyl aminopropyl)-3-ethyl-carbodiimide hydrochloride and methyl ethyl ketone are combined to provide a second mixture, S3-M2;
(c) combining the first mixture S3-M1 from step (a) with the second mixture S3-M2 from step (b) to provide a third mixture S3-M3; and
(d) stirring the third mixture S3-M3 from step (c) to provide the compound (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo [3.1.0]hexane-2-carboxamide, compound II.
   EMB-3 is the process of EMB-2 wherein in step (a) the first mixture, S3-M1, comprises 1.0 equivalents of (1R,2S,5S)-3-((S)-3,3-dimethyl-2-(2,2,2-trifluoroacetamido)butanoyl)-6,6-dimethyl-3-azabicyclo [3.1.0]hexane-2-carboxylic acid, compound IV, 2 L of methyl ethyl ketone per kg of compound IV, 0.9 equivalents of 2-Hydroxypyridine N-oxide and 2.50 equivalents of triethylamine.
EMB-4 is the process of EMB-2 or EMB-3 wherein in step (a) the first mixture, S3-M1, is prepared at about 25° C. and is stirred for about 30 minutes at about 25° C. and then is warmed to about 50° C.
EMB-5 is the process of any one of EMB-2 to EMB-4 wherein in step (b) the second mixture, S3-M2, comprises 1.05 equivalents of (S)-2-amino-3-((S)-2-oxopyrrolidin-3-yl)propanamide hydrochloride, compound III, 1.30 equivalents of 1-(3-dimethyl aminopropyl)-3-ethyl-carbodiimide hydrochloride and 3 L of methyl ethyl ketone per kg of compound IV.
EMB-6 is the process of any one of EMB-2 to EMB-5 wherein in step (b) the second mixture, S3-M2, is prepared at about 25° C. and is stirred for 30 minutes at about 25° C. and then is warmed to about 50° C.
EMB-7 is the process of any one of EMB-2 to EMB-6 wherein in step (c) the first mixture, S3-M1, from step (a) is at about 50° C. and is combined with the second mixture, S3-M2, from step (b) which is at about 50° C. to provide the third mixture, S3-M3, while maintaining the temperature of the third mixture, S3-M3, at about 50° C.
EMB-8 is the process of any one of EMB-2 to EMB-7 wherein in step (d) the third mixture, S3-M2, from step (c) is stirred for at least 6 hours at about 50° C.
EMB-9 is a process for preparing (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido) butanoyl)-6,6-dimethyl-3-azabicyclo[3.1.0]hexane-2-carboxamide, compound II

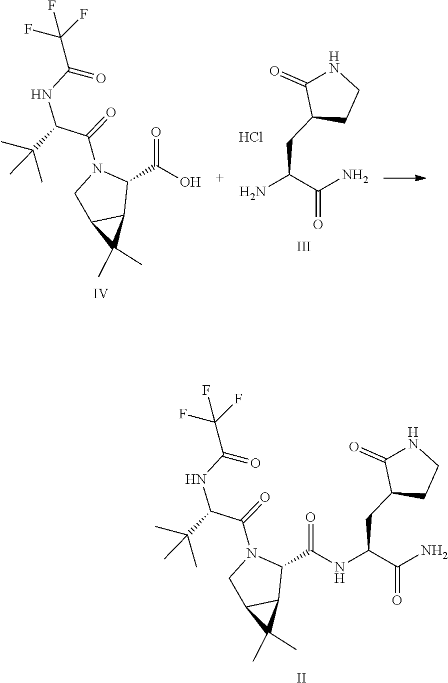

the process of reacting the compound of formula IV with the compound of formula III comprising the steps (a)-(d):
(a) (1R,2S,5S)-3-((S)-3,3-dimethyl-2-(2,2,2-trifluoroacetamido) butanoyl)-6,6-dimethyl-3-azabicyclo [3.1.0]hexane-2-carboxylic acid, compound IV (1.0 equivalent) and methyl ethyl ketone (2 L/kg of compound IV) at about 25° C. are combined followed by addition of 2-Hydroxypyridine N-oxide (0.90 equivalents) and triethylamine (2.50 equivalents), to provide a first mixture, S3-M1, which is stirred for about 30 minutes and then is warmed to about 50° C.;
(b) (S)-2-amino-3-((S)-2-oxopyrrolidin-3-yl)propanamide hydrochloride, compound III, (1.05 equivalents), 1-(3-dimethyl aminopropyl)-3-ethyl-carbodiimide hydrochloride (1.30 equivalents) and methyl ethyl ketone (3 L/kg of compound IV) are combined to provide a second mixture, S3-M2, which is stirred for about 30 minutes then warmed to about 50° C.;
(c) combining the first mixture, S3-M1, from step (a) with the second mixture, S3-M2, from step (b) while maintaining the temperature at about 50° C. to provide a third mixture, S3-M3; and
(d) stirring the third mixture, S3-M3, from step (c) at about 50° C. for at least 6 hours to provide the compound (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido) butanoyl)-6,6-dimethyl-3-azabicyclo [3.1.0]hexane-2-carboxamide, compound II.
EMB-10 is the process of EMB-9 wherein the amount of acylurea impurities IMP-S3-3, (2S,4S)-4-(2-aminoethyl)-5-oxopyrrolidine-2-carboxamide, and IMP-S3-4, (1R,2S,5S)-N-(2-((3S,5S)-5-carbamoyl-2-oxopyrrolidin-3-yl)ethyl)-3-((S)-3,3-dimethyl-2-(2,2,2-trifluoroacetamido)butanoyl)-6,6-dimethyl-3-azabicyclo [3.1.0]hexane-2-carboxamide formed is not more than 10%.
EMB-11 is the process of EMB-9 wherein the amount of acylurea impurities IMP-S3-3, (2S,4S)-4-(2-aminoethyl)-5-oxopyrrolidine-2-carboxamide, and IMP-S3-4, (1R,2S,5S)-N-(2-((3S,5S)-5-carbamoyl-2-oxopyrrolidin-3-yl)ethyl)-3-((S)-3,3-dimethyl-2-(2,2,2-trifluoroacetamido)butanoyl)-6,6-dimethyl-3-azabicyclo [3.1.0]hexane-2-carboxamide formed is not more than 5%.
EMB-12 is the process of any one of EMB-9 to EMB-11 wherein the amount of rearrangement impurity IMP-S3-3, (2S,4S)-4-(2-aminoethyl)-5-oxopyrrolidine-2-carboxamide formed is not more than 2%.
EMB-13 is the process of any one of EMB-9 to EMB-11 wherein the amount of rearrangement impurity IMP-S3-4, (1R,2S,5S)-N-(2-((3S,5S)-5-carbamoyl-2-oxopyrrolidin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide formed is not more than 2%.
EMB-14 is a process for preparing (1R,2S,5S)-3-((S)-3,3-dimethyl-2-(2,2,2-trifluoroacetamido)butanoyl)-6,6-dimethyl-3-azabicyclo [3.1.0]hexane-2-carboxylic acid, compound IV

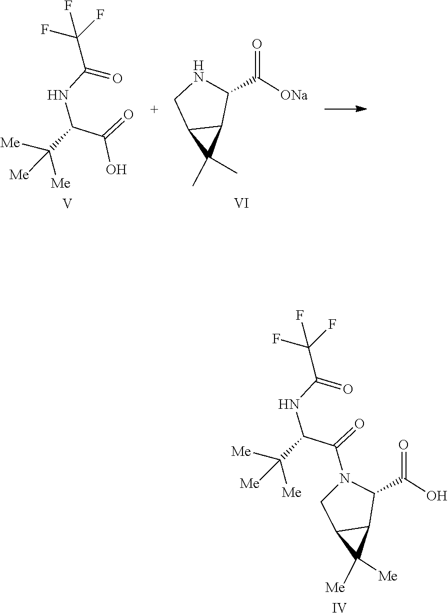

the process of reacting the compound of formula V with the compound of formula VI comprising the steps (a) to (c):
(a) combining(S)-3,3-dimethyl-2-(2,2,2-trifluoroacetamido)butanoic acid (compound V), methanesulfonyl chloride and isopropyl acetate to provide a first mixture, S2-M1;
(b) adding triethylamine to the first mixture, S2-M1, to provide a second mixture, S2-M2;
(c) adding Sodium (1R,2S,5S)-6,6-dimethyl-3-azabicyclo[3.1.0] hexane-2-carboxylate, compound VI, to the second mixture, S2-M2, to provide a third mixture, S2-M3; and
(d) stirring the third mixture, S2-M3, to provide (1R,2S,5S)-3-((S)-3,3-dimethyl-2-(2,2,2-trifluoroacetamido) butanoyl)-6,6-dimethyl-3-azabicyclo [3.1.0]hexane-2-carboxylic acid, compound IV.

EMB-15 is the process of EMB-14 wherein the first mixture, S2-M1, in step (a) comprises 1.2 equivalents of (S)-3,3-dimethyl-2-(2,2,2-trifluoroacetamido) butanoic acid, compound V, 1.1 equivalents of methanesulfonyl chloride and 20 mL of isopropyl acetate per g of compound V.
EMB-16 is the process of EMB-14 or EMB-15 wherein in step (b) 2.5 equivalents of triethylamine is added to the first mixture, S2-M1 which is at about 20° C., at a rate such that the temperature does not exceed 25° C. to provide the second mixture S2-M2.
EMB-17 is the process of any one of EMB-14 to EMB-16 wherein in step (c) 1.0 equivalents of Sodium (1R,2S,5S)-6,6-dimethyl-3-azabicyclo[3.1.0] hexane-2-carboxylate, compound VI, is added to the second mixture, S2-M2, to provide the third mixture S2-M3 which is stirred for about 4 hours.
EMB-18 is the process of EMB-17 wherein 2.5 equivalents of aqueous citric acid is added to the third mixture, S2-M3, and the resulting mixture is stirred for at least 10 minutes at about 40° C.
EMB-19 is the process of EMB-18 wherein the organic and aqueous layers of the resulting mixture are allowed to settle and the organic isopropyl acetate layer is separated from the aqueous layer, washed with water and concentrated to approximately 40% of its initial volume to provide organic layer, S2-M4.
EMB-20 is the process of EMB-19 wherein the organic layer S2-M4 is heated to 60° C. and to it is added one volume of heptane, then the resulting mixture is cooled to 10° C., stirred for 3 hours and the resulting solid is collected by filtration, washed with 1:1 isopropyl acetate/heptane and dried to provide (1R,2S,5S)-3-((S)-3,3-dimethyl-2-(2,2,2-trifluoroacetamido)butanoyl)-6,6-dimethyl-3-azabicyclo [3.1.0]hexane-2-carboxylic acid, IV.
EMB-21 is a process for preparing (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1, compound I

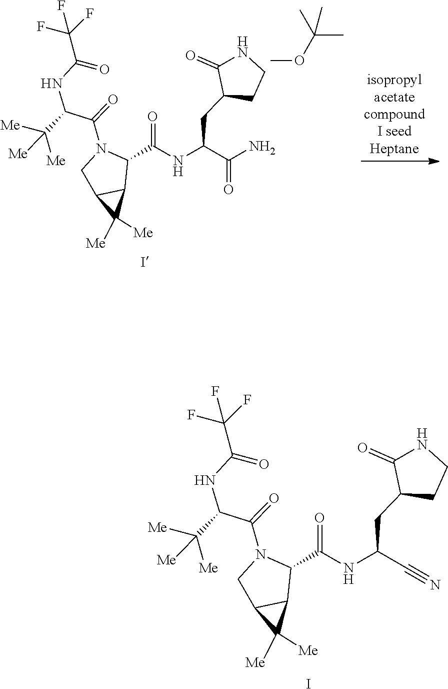

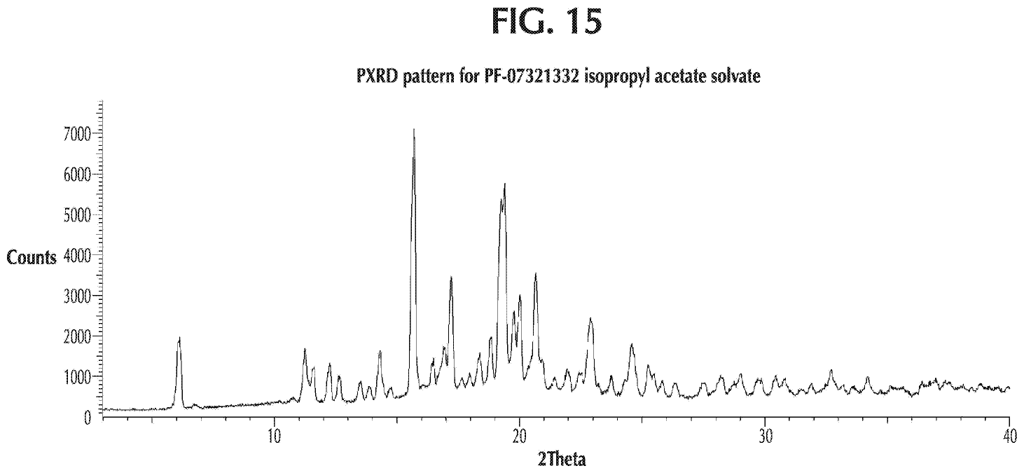

the process comprising the steps (a) to (d):
(a) dissolving (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, methyl tert-butyl ether, compound I′, in isopropyl acetate wherein the concentration of compound I′in isopropyl acetate is about 7 mL to about 9 mL of isopropyl acetate/1 gram of compound I′;
(b) seeding the solution with 0.5 weight % to 0.75 weight % of (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1
(c) adding heptane to the mixture from step (b) over a period of 6 to 15 hours wherein the amount of heptane added is about 10 mL to about 14 mL heptane/gram of compound I′; and
(d) isolating the resulting (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1.
EMB-22 is the process of EMB-22 wherein the amount of isopropyl acetate used in step (a) is about 8 mL of isopropyl acetate/gram of compound I′.
EMB-23 is the process of EMB-21 or EMB-22 wherein the amount of (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1 used to seed the solution in step (b) is about 0.75 w %.
EMB-24 is the process of any one of EMB-21 to EMB-23 wherein the amount of heptane added in step (c) is about 12 mL/gram of compound I′.
EMB-25 is the process of any one of EMB-21 to EMB-24 wherein the heptane is added over a period of about 10 hours.
EMB-26 is the process of any one of EMB-21 to EMB-25 wherein the (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1 is isolated by filtration.
EMB-27 is the process of any one of EMB-21 to EMB-26 wherein the (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1 isolated in step (d) has a particle size distribution with a D[v, 0.5] count of about 12 microns to about 18 microns.
EMB-28 is the process of any one of EMB-21 to EMB-27 wherein the (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1 isolated in step (d) has a particle size distribution with a D[v, 0.5] count of about 14 microns to about 16 microns.
EMB-29 is the process of any one of EMB-21 to EMB-28 wherein the (1R,2S,5S)-N-{(1S)-1-cyano-2-[(3S)-2-oxopyrrolidin-3-yl]ethyl}-6,6-dimethyl-3-[3methyl-N-(trifluoroacetyl)-L-valyl]-3-azabicyclo[3.1.0]hexane-2-carboxamide, Form 1 isolated in step (d) has a particle size distribution with a D[v, 0.5] count of about 15 microns.
EMB-30 is the compound (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0] hexane-2-carboxamide, isopropyl acetate solvate.
EMB-31 is the compound of EMB-30 wherein the compound is crystalline.
EMB-32 is the compound of EMB-31 which is characterized by the PXRD pattern as depicted in FIG. 15.

### Preparation of Starting Materials and Intermediates

Synthetic routes for the preparation of the intermediate compound of formula 1 is provided in Reaction Schemes A and B and for the preparation of the compound of formula 2 in Reaction Schemes C, D and E.

The compound of Formula 1 may be prepared by the following methods as described below and depicted in Reaction Schemes A and B. For the compound of formula 1 it is to be understood that when R¹ is methyl and the compound is in the form of its hydrochloride salt the compound is methyl (1R,2S,5S)-6,6-dimethyl-3-azabicyclo[3.1.0]hexane-2-carboxylate, hydrochloride salt which is also referred to as compound VII in Reaction Scheme 1.

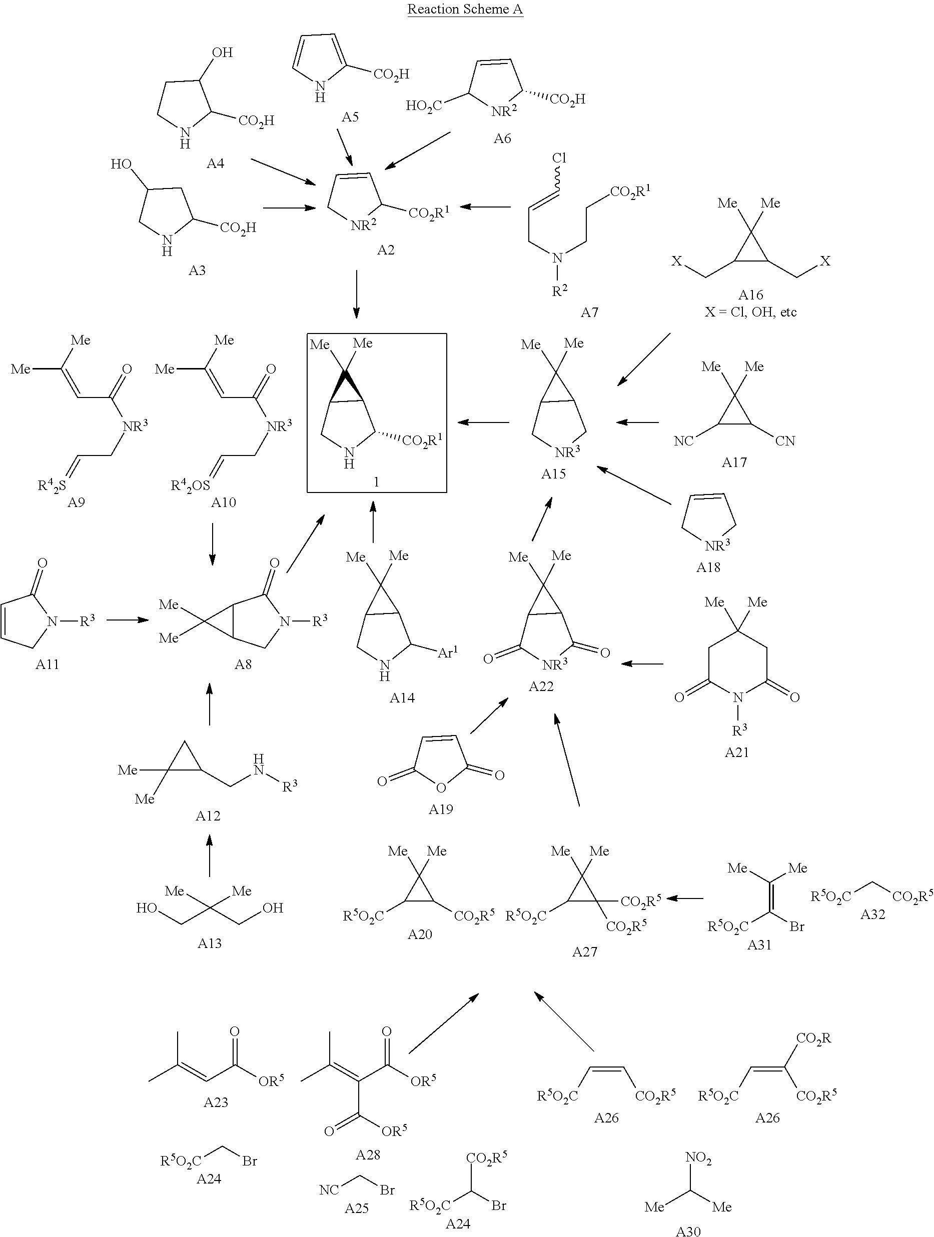

In Reaction Scheme A the variables R¹, R⁴ and R⁵ represent alkyl groups including, but not limited to, methyl, ethyl and isopropyl groups. The variable Ar¹ represents an aryl group including, but not limited to, a 2-methoxyphenyl group. R² and R³ represent amine protecting groups that are well-known to those skilled in the art. (see for example Wuts, P. G. M; Greene, T. W. Greene's Protective Groups in Organic Synthesis, 4^{th} ed.; John Wiley & Sons, Inc., 2006.

The compound of formula 1 may be prepared from cyclopropanation of A2 followed by deprotection. Compound A2 may be prepared from A3 or A4 by conversion of the hydroxyl group of the A3 or A4 compound into an activated group with reactions with a reagent including, but not limited to, tosyl chloride, mesyl chloride, triflic anhydride and sodium iodide, followed by elimination with a base to provide the compound A2. The R¹ group may be installed before (i.e. by replacing the hydrogen of the carboxylic acid group in A3 or A4 with R¹) or after this step using a method well known to those skilled in the art. A2 may also be prepared by a reduction of the pyrrole A5 or decarboxylation of A6. A2 may also be prepared by an intermolecular cyclization of A7 under the conditions including, but not limited, to metal catalyzed alpha vinylation.

The compound of formula 1 may also be prepared from a functionalization of A8 via transformations including, but not limited to, reduction of amide to imine followed by cyanation and esterification. These transformations may require a chemical or enzymatic catalyst(s). A8 may be formed via intramolecular cyclopropanation of A9 or A10, by cyclopropanation of A11, or by a metal catalyzed carbonylative C—H functionalization of A12 and A12 may be prepared from A13. The compound of formula 1 may be prepared from oxidation of A14 to a carboxylic acid followed by esterification. The transformation may require a metal catalyst. The compound of formula 1 may be prepared via a functionalization of A15 via transformations including, but not limited to, metalation with an organometallic reagent followed by carboxylation and borylation followed by carboxylation. A15 may be prepared from reactions including, but not limited to, coupling of A16 with an amine source, reduction and cyclization of A17, cyclopropanation of A18, or reduction of A22. A22 can be synthesized by cyclopropanation and aminolysis of A19, aminolysis and cyclization of A20, aminolysis, cyclization and decarboxylation of A27, or oxidative ring contraction of A21. A20 or A27 may be formed from a reaction of A23 or A28 with A24, A25 or A33 in presence of dialkylsulfide or cyclopropanation of A26 or A29 with cyclopropanating reagents including, but not limited to, A30. A27 may also be prepared by reactions between A31 and A32 in presence of a base.

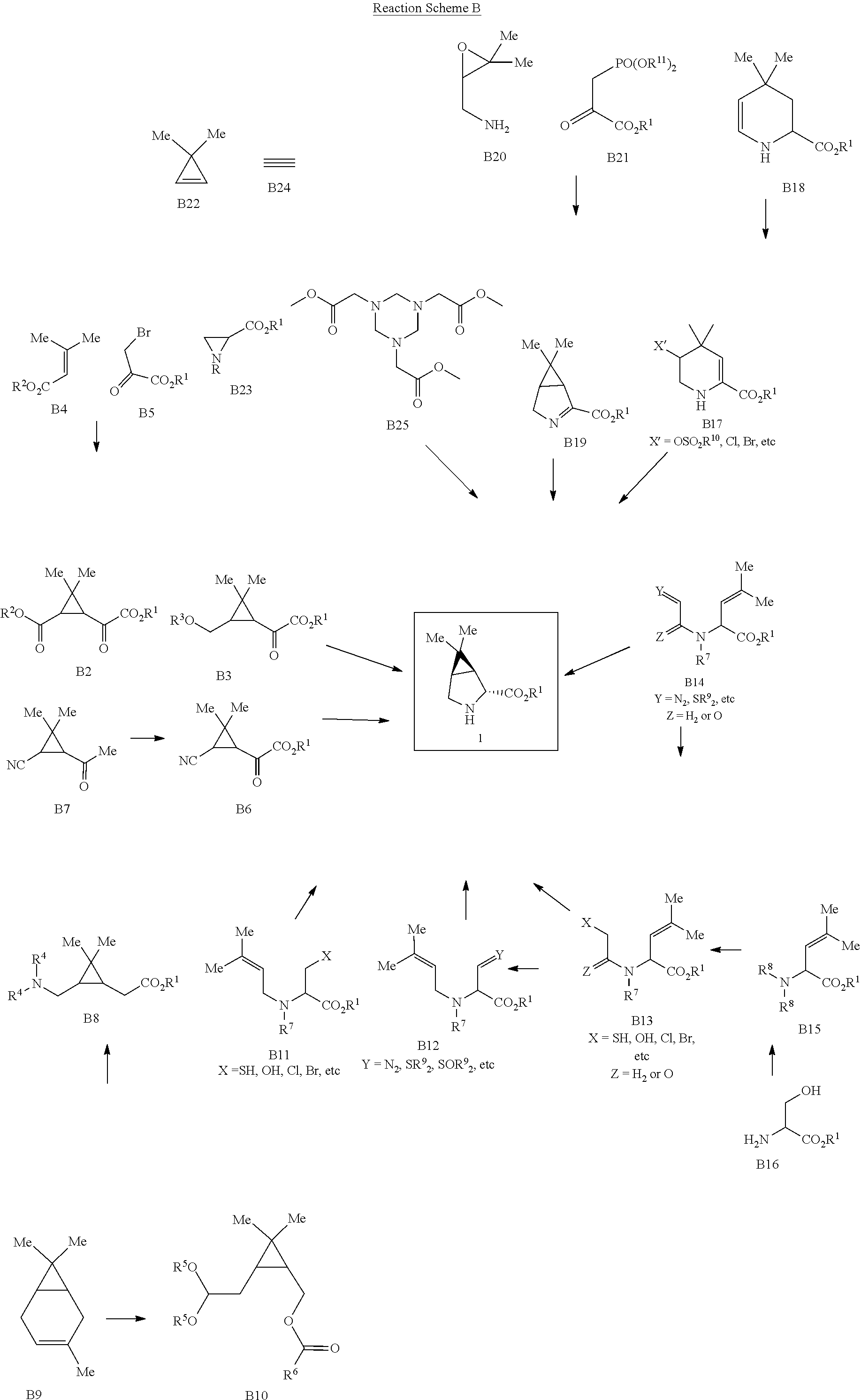

In Reaction Scheme B, the variables R¹, R², R³, R⁵, R⁶, R⁹ and R¹⁰ represent alkyl or aryl groups including, but not limited to, methyl, ethyl, isopropyl and tolyl groups. The variables R⁴, R⁷ and R⁸ represent amine protecting groups that are well-known to those skilled in the art.

The compound of formula 1 may be prepared by cyclization of B2 and B3 with an ammonia source in the presence of a chemical or enzymatic catalyst(s) and a reducing reagent. The resulting product may be subjected to another reduction reaction if necessary. B2 may be prepared by a coupling of B4 and B5.

The compound of formula 1 may be prepared from cyclization of B6 under reducing conditions in presence of a chemical or enzymatic catalyst(s). B6 may be prepared from B7 under conditions well known to those skilled at the art. B8 may be treated with a chlorination reagent in presence of a base to form the compound of formula 1. B8 may also be prepared from B9 via B10 by transformations including, but not limited to, ozonolysis.

The compound of formula 1 may be prepared from an intramolecular cyclopropanation of B11, B12, B13 or B14. This cyclopropanation reaction may require a chemical and/or enzymatic catalyst(s) and the resulting product may need to be reduced to form the compound of formula 1. B12 may be prepared from B11 and B14 may be prepared from B13. B16 may undergo oxidation and olefination to form B15, which then may be transformed to B13 under conditions well known to those skilled at the art. The compound of formula 1 may also be prepared from intramolecular carbon-carbon bond formation of B17 under a reducing condition and B17 may be formed from functionalization (i.e. introduction of the group OSO₂R¹⁰, Cl, Br, etc. with double bond rearrangement) of B18. B20 and B21 may be reacted in presence of a base to form B19, which may be then be reduced to compound of formula 1 in the presence of a chemical and/or enzymatic catalyst(s). B22 or B24 may be reacted with azomethine ylides derived from B23 or B25 to form either the compound of formula 1 or A2, and A2 may then be cyclopropanated to form the compound of formula 1.

The compound of formula 2 may be synthesized by the following methods as described in Scheme C, D and E.

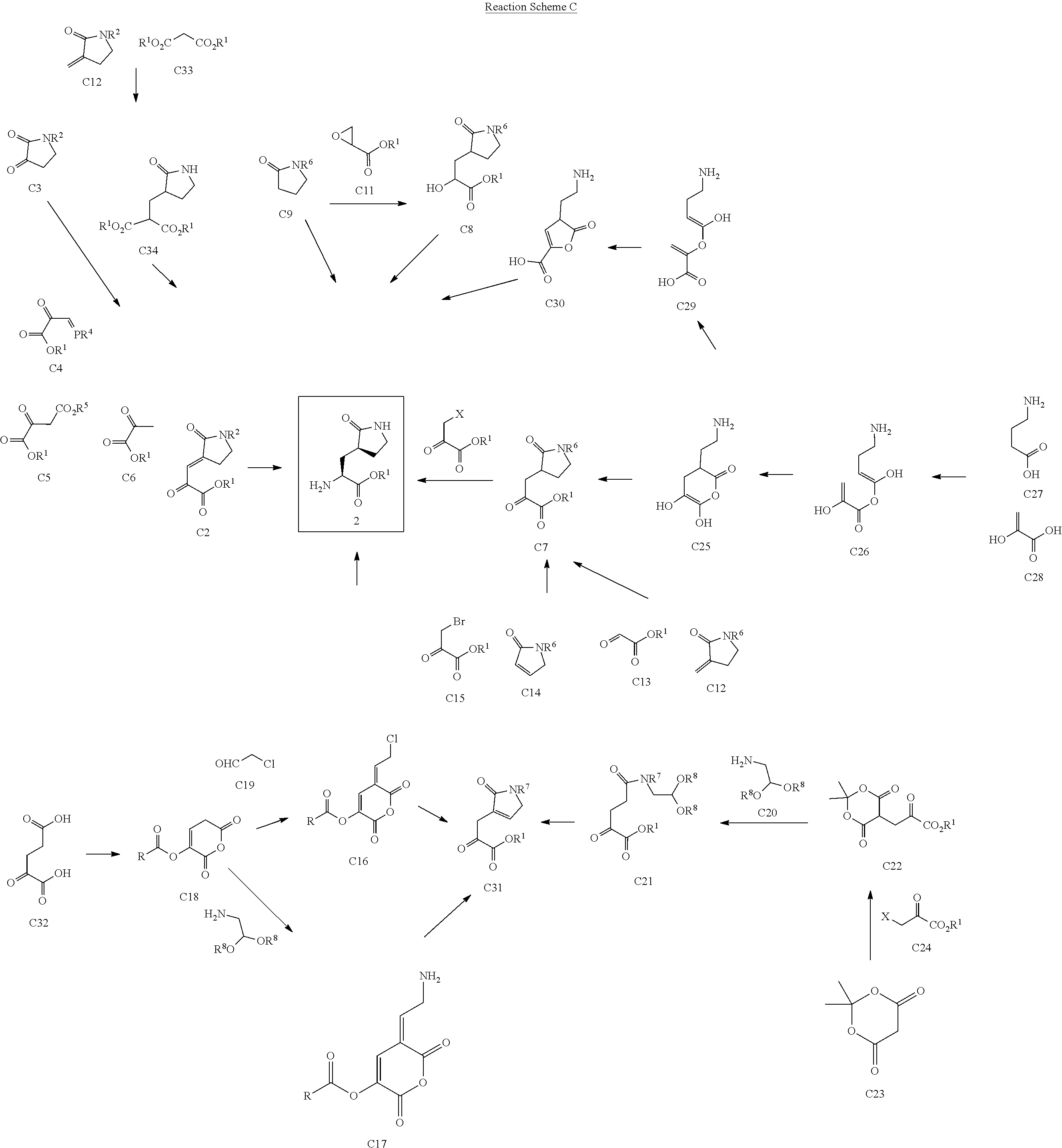

In Scheme C, the variables R¹, R⁴, R⁵ and R⁸ represent alkyl or aryl groups including, but not limited to, methyl, ethyl, isopropyl and tolyl groups. The variables R², R⁶ and R⁷ represent amine protecting groups that are well-known to those skilled in the art.

The compound of formula 2 may be prepared by a conversion of compound C2 by reductive amination of the ketone moiety and reduction of the enone moiety of C2. This conversion may require a chemical or enzymatic catalyst(s). The compound C2 may be prepared from the reactions including, but not limited to, Wittig reaction of compound C3 with compound C4 or aldol reactions of compound C3 with compound C5 or C6 followed by decarboxylation if needed under conditions well known to those skilled in the art. Compound C12 may be reacted with C33 to form C34 in the presence of a base. C34 may undergo stereoselective hydrolysis of ester (desymmetrization wherein one of the R¹ is then hydrogen) in presence of chemical or enzymatic catalyst. The resulting acid moiety may be reacted with a nitrogen source to prepare an amide, which then may be reacted under the Curtius, Lossen or Hofmann rearrangement conditions to yield compound 2.

The compound of formula 2 may also be prepared by a reductive amination of compound C7 and this conversion can require a chemical or enzymatic catalyst(s). Compound C7 may be prepared from alkylation of compound C9 with compound C10 or alkylation of compound C9 with compound C11 followed by oxidation of the resulting product C8. This conversion may require a chemical or enzymatic catalyst(s). Compound C7 may also be prepared from a reaction of compound C12 with compound C13 in presence of a thiazolium salt or a reaction of compound C14 with compound C15 in presence of a catalyst such as tertiary amines and phosphines. Compound C7 may also be prepared from a coupling of C27 and C28 to form C26 or C29, followed by rearrangements via C25 or C30.

The compound of formula 2 may also be prepared by a conversion of compound C31 by reductive amination of the ketone moiety and reduction of the enone moiety of C31. This conversion may require a chemical or enzymatic catalyst(s). Compound C31 may be prepared from C16 in presence of ammonia or from C17 in presence of acid or base. Compounds C16 and C17 may be prepared from reactions of compound C18 with C19 or C20, respectively, under the conditions well known to those skilled in the art. Compound C18 may be prepared from compound C32 in presence of a reagent including, but not limited to, acetic anhydride. Compound C31 may also be prepared from Compound C21 under the conditions involving acid or base. Compound C21 may be prepared from alkylation of C23 with C24 followed by reaction with C20 and decarboxylation.

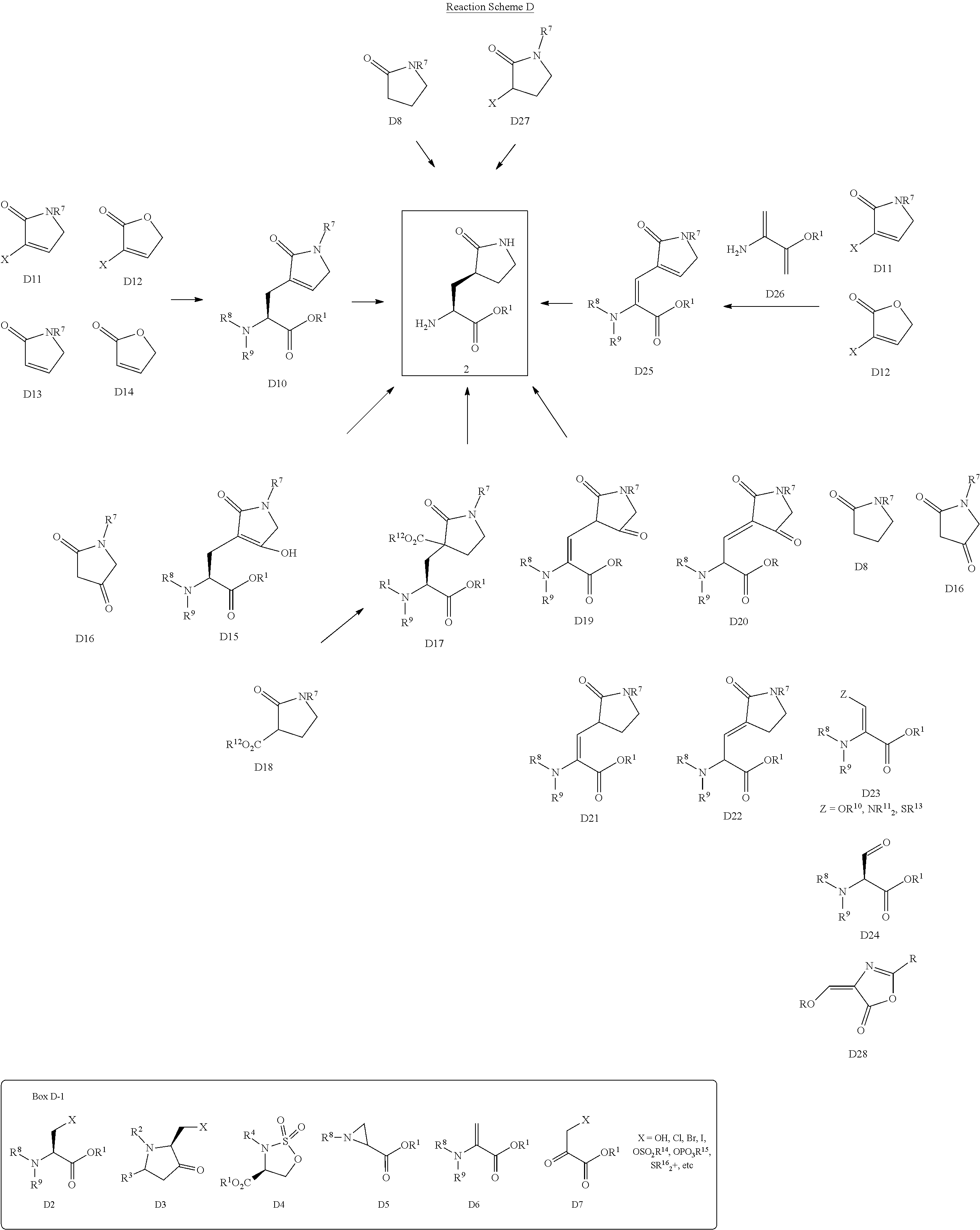

In Reaction Scheme D the variables R¹, R³, R¹⁰, R¹³, R¹⁴, R¹⁵ and R¹⁶ represent alkyl or aryl groups including, but not limited to, methyl, ethyl, isopropyl and tolyl groups. The variables R², R⁴, R⁵, R⁷, R⁸, R⁹ and R¹¹ represent amine protecting groups that are well-known to those skilled in the art.

The compound of formula 2 may be prepared by reactions of compound D8 with partners including, but not limited to, compounds D2, D3, D4, D5, D6 and D7. The conversions may require components including, but not limited to, chemical or enzymatic catalysts and acids or bases. One skilled in the art will appreciate that the resulting products from these transformations may require further transformations. For example, the resulting product from alkylation of D8 with D7 may require reductive amination in presence of a chemical catalyst or an enzyme.

The compound of formula 2 may be prepared by a reduction of compound D10 in presence of an enzyme(s) or a chemical catalyst(s), followed by deprotection if necessary. Compound D10 may be prepared by a reaction of compound D11 or D12 with D2, D3 or D7 in presence of a metal catalyst and zinc or other metals as a stoichiometric reagent, followed by additional transformations such as aminolysis, reductive amination and/or deprotection. Compound D10 may also be prepared by reactions of D13 or D14 with D2, D3 or D7 in presence of a metal or organic catalyst(s) followed by additional transformations such as aminolysis, reductive amination, and/or deprotection.

The compound of formula 2 may be prepared by reduction of compound D15. This conversion may require a chemical or enzymatic catalyst(s). D15 may be prepared by a reaction of D16 with D2, D3, D4, D5, D6 or D7 in the presence of a chemical catalyst(s), an enzyme(s) and/or a base(s), followed by additional transformations such as reductive amination, and/or deprotection. The compound of formula 2 may be prepared by decarboxylation of compound D17 in presence or absence of a catalyst. Compound D17 then may be prepared by a reaction of D18 with D2, D3, D4, D5, D6 or D7 in the presence of a chemical catalyst(s), an enzyme(s) and/or a base(s), followed by additional transformations such as reductive amination, and/or deprotection.

The compound of formula 2 may be prepared by a reduction of compound D19, D20, D21 or D22. This conversion may require a chemical or enzymatic catalyst(s). D19 and D20 may be prepared by a reaction of D16 with D23 or D24 in the presence of acid or base. D21 and D22 may be prepared by a reaction of D8 with D23 or D24 in the presence of acid or base. Alternatively, D8 or D16 may be reacted with D28 in the presence of a base followed by alcoholysis with R¹OH to produce D19, D20, D21 or D22.

The compound of formula 2 may be prepared by a reduction of compound D25. This conversion may require a chemical or enzymatic catalyst(s). Compound D25 may be prepared by a reaction of D11 with D26 in presence of a metal catalyst or a reaction of D12 with D26 in presence of a metal catalyst followed by aminolysis.

The compound of formula 2 may be prepared by a reaction of compound D27 with compound D2, D3, D4, D5, D6 or D7 in the presence of a reductant including but not limited to zinc and manganese and a metal catalyst, followed by additional transformations including, but not limited to, deprotection and reductive amination.

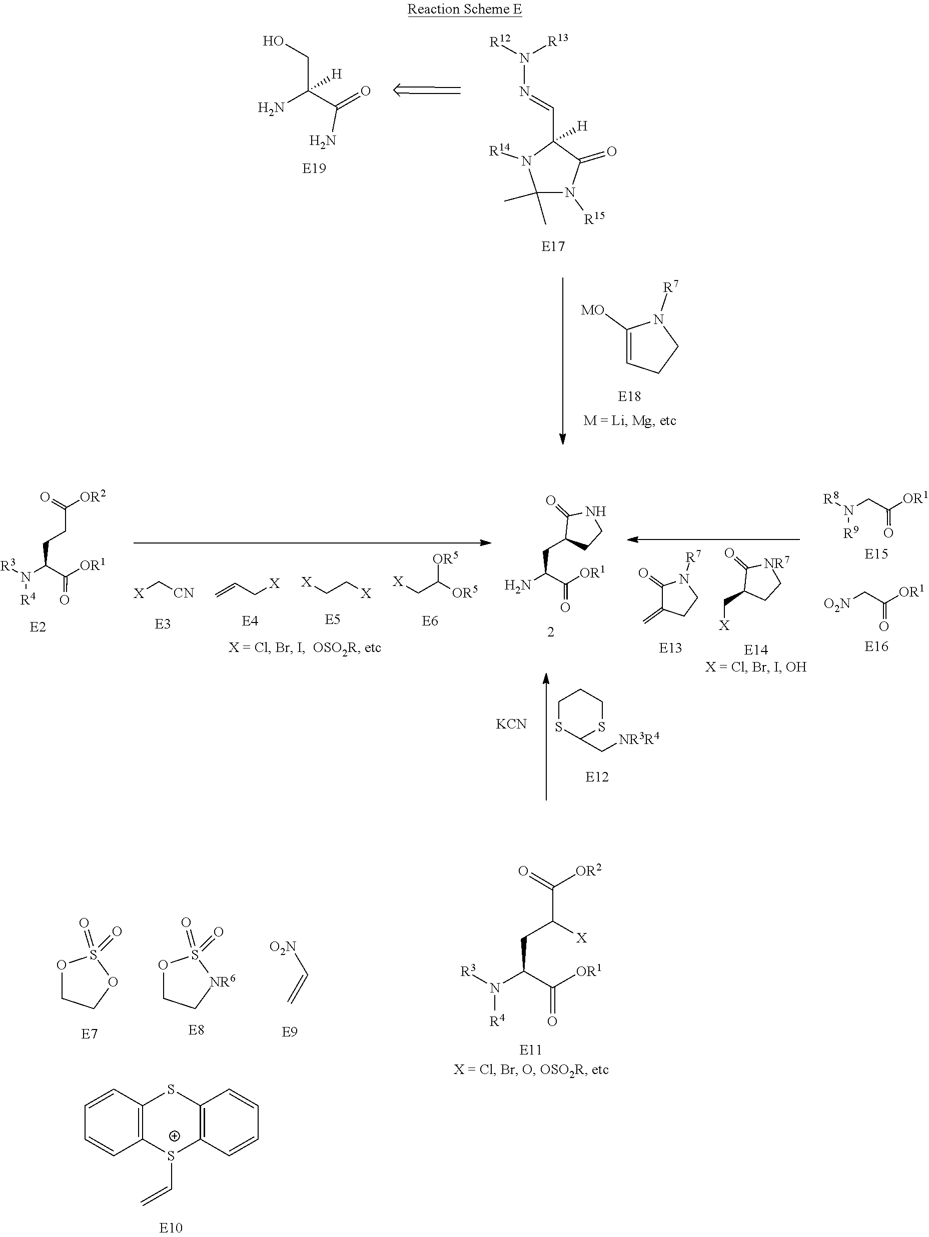

In Scheme E, R¹, R², R⁵, and R¹⁶ groups are alkyl or aryl group including, but not limited to, methyl, ethyl, isopropyl, and tolyl groups. R³, R⁴, R⁶, R⁷, R⁸, R⁹, R¹², R¹³, R¹⁴, R¹⁵, and R¹⁷ groups are protecting groups that are well-known to those skilled in the art.¹

N-protected glutamic acid ester E2 may be reacted with a base, and the resulting anion may be reacted with a reagent including, but not limited to, E3, E4, E5, E6, E7, E8, E9, and E10. Resulting products may then be converted to 2 by using methods well known to those skilled in the art.

N-protected glutamic acid ester E20 may be reacted with a base, and the resulting product may be reacted with a reagent including, but not limited to, E3, E4, E5, E6, E7, E8, E9, and E10. The alkylation may or may not be stereoselective. The resulting product may undergo reductive amination in presence of chemical or enzymatic catalyst such as transaminase. Resulting products may then be converted to 2 by using methods well known to those skilled in the art.

E2 may be converted to E23 via amidation. E23 may then be reacted with a base to yield Compound 2.

Compound 2 may be prepared by rearrangement and deprotection (if needed) of E24. E24 may be prepared by cyanide addition to E25 followed by alkene and nitrile reduction or reduction and deprotection of E26. E25 or E26 may be prepared from E27 under the conditions well known to those skilled in the art.

E28 or E29 may be converted to Compound 2 under the conditions well known to those skilled at art. For example, E28 may be subjected to an olefin reduction condition in presence of a chemical or enzymatic catalyst such as, but not limited to, Ene Reductase, followed by saponification of ester, asymmetric decarboxylation and reduction of nitrile to yield E30, which may be performed in presence of a chemical or enzymatic catalyst. Alternatively, E29 may be subjected to reductions of olefin and cyano groups in presence of a chemical and enzymatic catalyst to yield E30. Intramolecular cyclization of E30 may then produce Compound 2 after deprotection is performed if needed.

Compound 2 may be converted to Compound E11 by a method including, but not limited to, halogenation with N-halosuccinimide and enzymatic halogenation. Compound E11 may then be reacted with a reagent including, but not limited to, E12 and KCN. Resulting product may then be converted to 2 by using methods well known to those skilled in the art.

Compound E15 or E16 may be reacted with a reagent such as E13 or E14 in presence of a base and/or a chiral catalyst to form 1.

Compound E22 may be subjected to Strecker reaction conditions, well known to those skilled in art, to prepare E21, which then can be transformed into 1 under the conditions, well known to those skilled at art. Synthesis of E21 may be performed in a stereoselective manner; otherwise, conversion from E21 to 1 may be performed via enzymatic resolution or dynamic kinetic asymmetric transformation.

Compound 2 may be prepared by a reaction of E17 and E18 followed by deprotections, if necessary. E17 may be prepared from E19 via methods well known to those skilled in the art.

### Process for the Preparation of Nirmatrelvir

In Step 2 of Reaction Scheme 1 it was found that under certain reaction conditions impurities such as (S)-N,N-diethyl-3,3-dimethyl-2-(2,2,2-trifluoroacetamido)butanamide (denoted as diethylamide impurity, IMP-S2-1), (1R,2S,5S)-3-((R)-3,3-dimethyl-2-(2,2,2-trifluoroacetamido)butanoyl)-6,6-dimethyl-3-azabicyclo[3.1.0]hexane-2-carboxylic acid (denoted as epimer impurity, IMP-S2-2) and N-((S)-1-((1R,2S,5S)-2-((1R,5S)-6,6-dimethyl-3-azabicyclo[3.1.0]hexane-3-carbonyl)-6,6-dimethyl-3-azabicyclo[3.1.0]hexan-3-yl)-3,3-dimethyl-1-oxobutan-2-yl)-2,2,2-trifluoroacetamide (denoted as bisamide impurity, IMP-S2-3) were formed (see Reaction Scheme below). Advantageously, the present invention minimizes the formation of these undesired impurities. The mechanism of formation of the(S)-N, N-diethyl-3,3-dimethyl-2-(2,2,2-trifluoroacetamido) butanamide, IMP-S2-1, is unclear but is unrelated to the presence of diethylamine in the triethylamine (TEA) used in the reaction. The diethylamide impurity was observed to form when the methanesulfonyl choride was added to the mixture of (S)-3,3-dimethyl-2-(2,2,2-trifluoroacetamido)butanoic acid, compound V, in the presence of triethylamine or when a larger excess of triethylamine (3.0 equivalents vs. 2.5 equivalents of TEA) was used in the reaction. In both instances, the amount of diethylamide impurity and bisamide impurity are minimized by addition of the methanesulfonyl chloride to the compound of Formula V in isopropyl acetate prior to the addition of the triethylamine. The use of methanesulfonyl chloride and TEA was also found to be advantageous as minimal epimerizaton occurred when using those reagents in the amidation reaction. The present invention therefore provides significant advantages as the claimed Step 2 process minimizes the formation of the undesired diethyl amide and bisamide impurities, IMP-S2-1 and IMP-S2-3, by controlling the order of addition of the methane sulfonyl chloride to the reaction mixture and also minimizes the amount of epimerized product, IMP-S2-2, formed by control of the amount of triethylamine base used.

### Step 2 of Reaction Scheme 1 With Potential Impurities

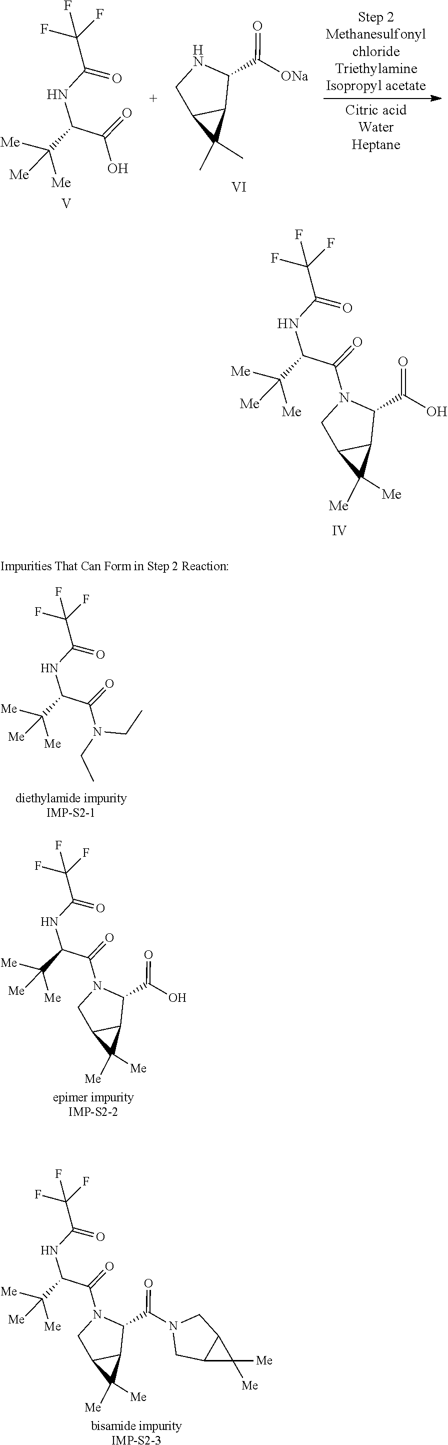

In Step 3 of the Reaction Scheme 1 process it was found that under certain reaction conditions acylurea impurities as well as rearrangement related impurities can form. The structure of the acylurea impurities that can form in Step 3 are depicted below as IMP-S3-1 and IMP-S3-2. IMP-S3-1 is (1R,2S,5S)-3-((S)-3,3-dimethyl-2-(2,2,2-trifluoroacetamido)butanoyl)-N-(3-(dimethylamino)propyl)-N-(ethylcarbamoyl)-6,6-dimethyl-3-azabicyclo[3.1.0]hexane-2-carboxamide and IMP-S3-2 is (1R,2S,5S)-3-((S)-3,3-dimethyl-2-(2,2,2-trifluoroacetamido)butanoyl)-N-((3-(dimethylamino)propyl) carbamoyl)-N-ethyl-6,6-dimethyl-3-azabicyclo[3.1.0]hexane-2-carboxamide. Under certain reaction conditions it was found that(S)-2-amino-3-((S)-2-oxopyrrolidin-3-yl)propenamide can undergo a rearrangement reaction to form the rearrangement impurity IMP-S3-3 which can further react under the Step 3 amidation reaction conditions to form an additional impurity designated IMP-S3-4. IMP-S3-3 is (2S,4S)-4-(2-aminoethyl)-5-oxopyrrolidine-2-carboxamide and IMP-S3-4 is (1R,2S,5S)-N-(2-((3S,5S)-5-carbamoyl-2-oxopyrrolidin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide. Minimizing the amount of IMP-S3-4 formed is desirable to avoid further carry through of this impurity and subsequent reaction under the Step 4 reaction conditions wherein the amido moiety on the lactam ring of IMP-S3-4 can be converted into a nitrile moiety forming the impurity designated IMP-S4-1 which is (1R,2S,5S)-N-(2-((3S,5S)-5-cyano-2-oxopyrrolidin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide.

### Step 3 Reaction With Potential Impurities Formed in Step 3 and Step 4 of Reaction

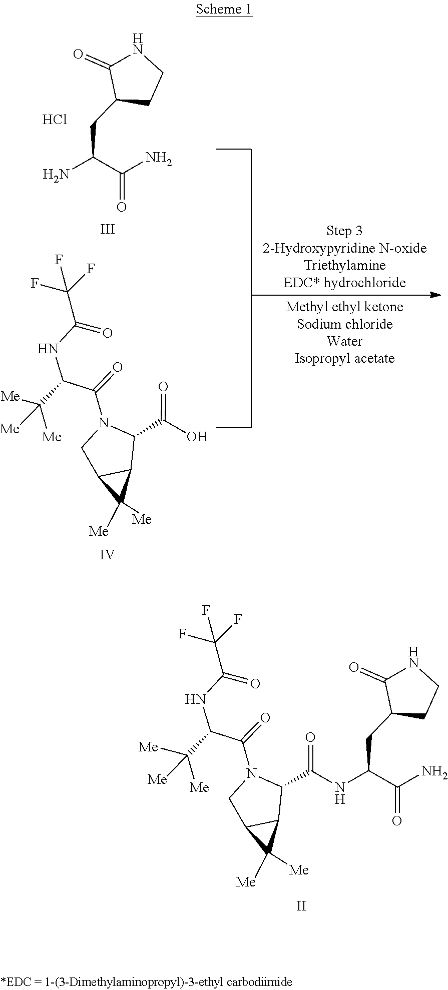

### Acylurea Impurities Formed in Step 3

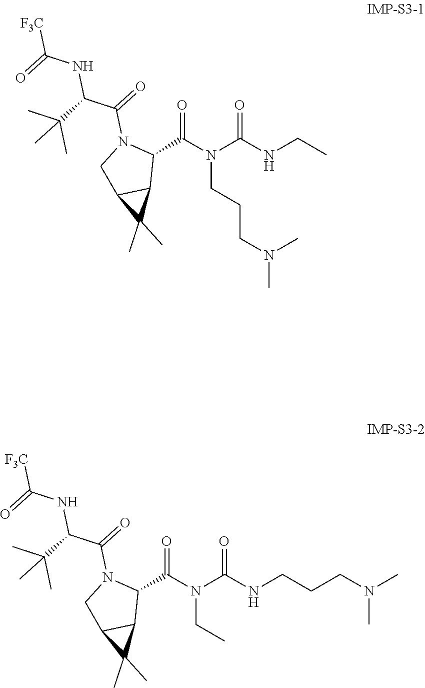 [SMILES: CCNC(=O)N(CCCN(C)C)C(=O)[C@@H]1[C@@H]2[C@H](CN1C(=O)[C@@H](NC(=O)C(F)(F)F)C(C)(C)C)C2(C)C ; CCN(C(=O)NCCCN(C)C)C(=O)[C@@H]1[C@@H]2[C@H](CN1C(=O)[C@@H](NC(=O)C(F)(F)F)C(C)(C)C)C2(C)C]

### Rearrangement By-Product From Step 3 and Products Formed in Step 3 and Step 4

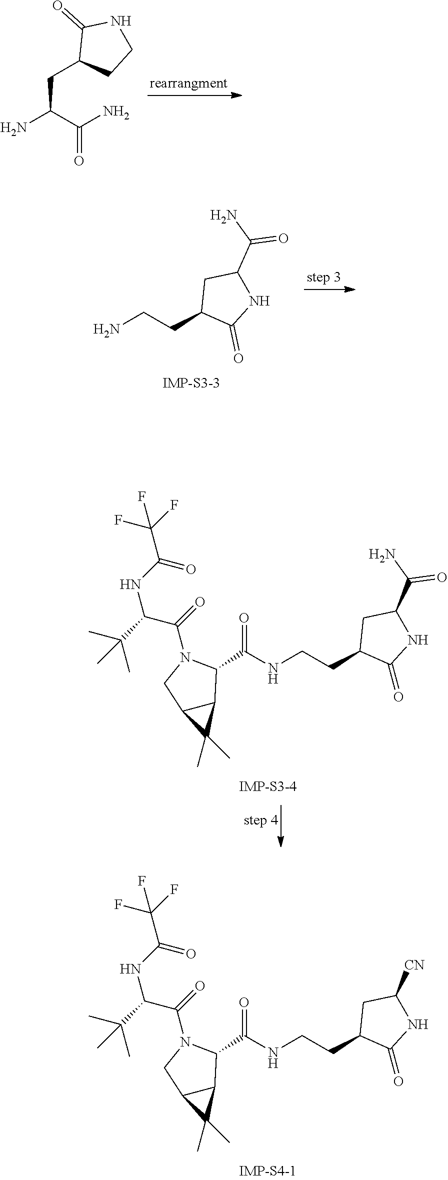 [SMILES: NC(=O)[C@@H](N)C[C@@H]1CCNC1=O ; NCC[C@H]1CC(C(N)=O)NC1=O ; CC(C)(C)[C@H](NC(=O)C(F)(F)F)C(=O)N1C[C@H]2[C@@H]([C@H]1C(=O)NCC[C@H]1C[C@@H](C(N)=O)NC1=O)C2(C)C ; CC(C)(C)[C@H](NC(=O)C(F)(F)F)C(=O)N1C[C@H]2[C@@H]([C@H]1C(=O)NCC[C@H]1C[C@@H](C#N)NC1=O)C2(C)C]

In Step 3 it was found that the use of non-recrystallized (S)-2-amino-3-((S)-2-oxopyrrolidin-3-yl)propanamide hydrochloride, compound III or recrystallized (S)-2-amino-3-((S)-2-oxopyrrolidin-3-yl)propanamide hydrochloride, compound III had an impact on the amount of the acylurea impurities IMP-S3-1 and IMP-S3-2 as shown in column 4 of Table: Step 3 Reaction Conditions below. When non-recrystallized (S)-2-amino-3-((S)-2-oxopyrrolidin-3-yl)propanamide hydrochloride, compound IlI was used in the two reactor set-up at 50° C. not more than 5% of the acylurea impurities IMP-S3-1 and IMP-S3-2 were formed, not more than 2% of the rearrangement impurity IMP-S3-3 was formed with favorable reaction kinetics (less than 3% of starting compound IV remains after ˜2 hours reaction time). When recrystallized (S)-2-amino-3-((S)-2-oxopyrrolidin-3-yl)propanamide hydrochloride, compound IlI was used in the two reactor set-up at 50° C. not more than 10% of the acylurea impurities IMP-S3-1 and IMP-S3-2 were formed, not more than 2% of the rearrangement impurity IMP-S3-3 was formed with favorable reaction kinetics (less than 3% of starting compound IV remains after ˜6 hours reaction time). The two-reactor process when run at 50° C. provides favorable reaction kinetics while maintaining relatively low levels of the acylurea and rearrangement impurities that can form. The initial process conditions (row 2 of table below) suffered from less than desired reaction kinetics (16 hours or 60-100 hours reaction time depending on the type of compound III employed) while the one reactor process when run at 50° C. resulted in large amounts of rearrangement impurity being formed (see row 3 of table, not less than 50% or not less than 40% or rearrangement impurity being formed).

TABLE
Step 3 Reaction Conditions

| | | | Percent | Reaction | | |
|---|---|---|---|---|---|---|
| | | | Acylurea | Kinetics | | Percent |
| | | | Impurities | (time to reach | | Rearrangement |
| | | Type of | IMP-S3-1 | not more | | impurity |
| | Number of | Compound III | and IMP-S3-2 | than 3% of | | IMP-S3-3 |
| Procedure | Reactors | Used in Step 3 | Formed | compound IV) | | Formed |
| Initial | 1 | Non- | Not more | 16 | hours | Not more |
| Process | Reactor | recrystallized | than 2% | | | than 1% |
| HOPO: 0.75 | | Recrystallized | ~20% | 60-100 | hours | Not more |
| equivalents; | | | | | | than 2% |
| TEA: 1.5 | | | | | | |
| equivalents; | | | | | | |
| Temperature: | | | | | | |
| 25° C. | | | | | | |
| Optimized | 1 | Non- | Not more | ~2 | hours | Not less |
| Process | Reactor | recrystallized | than 5% | | | than 50% |
| HOPO: 0.90 | | Recrystallized | Not more | ~6 | hours | Not less |
| equivalents; | | | than 10% | | | than 40% |
| TEA: 2.5 | | | | | | |
| equivalents; | | | | | | |
| Temperature: | | | | | | |
| 50° C. | | | | | | |
| Optimized | 2 | Non- | Not more | ~2 | h | Not more |
| Process | Reactors | recrystallized | than 5% | | | than 2% |
| HOPO: 0.90 | | Recrystallized | Not more | ~6 | h | Not more |
| equivalents; | | | than 10% | | | than 2% |
| TEA: 2.5 | | | | | | |
| equivalents; | | | | | | |
| Temperature: | | | | | | |
| 50° C. | | | | | | |

### Step 5: Nirmatrelvir API Crystallization Process With Optimal Particle Size Control

The crystallization of PF-07321332 anhydrous Form 1, compound I, begins with a solution of PF-07321332 MTBE solvate, compound I′, in isopropyl acetate solvent followed by seeding with anhydrous form 1 solids and addition of heptane as an antisolvent. It is this step, that is important in controlling particle size, and polymorphic Form. Therefore, the crystallization and isolation of PF-07321332 is an important process to control API physical properties for optimal drug product performance.

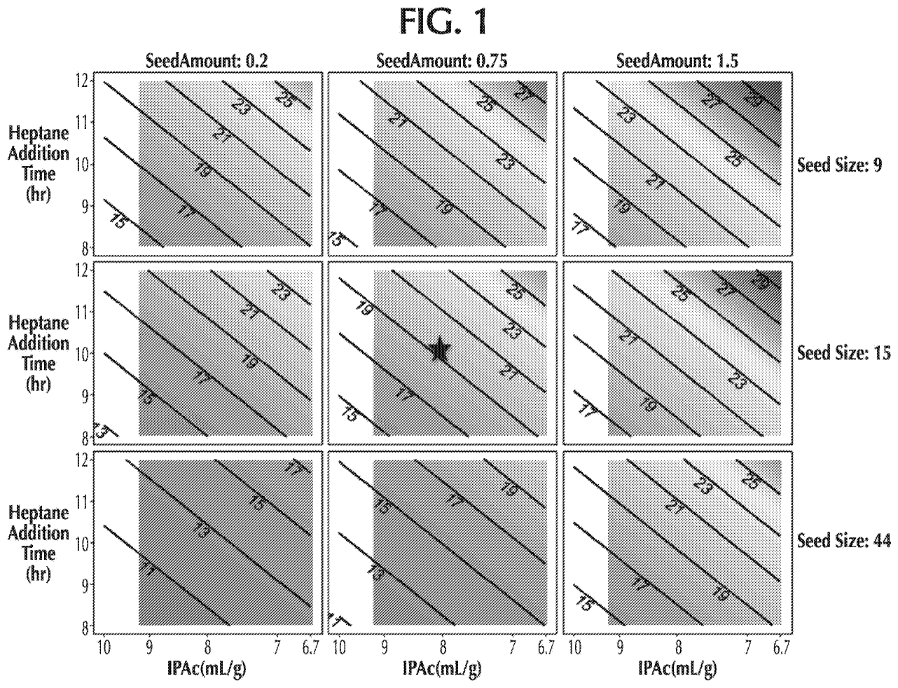

The initial concentration of API (PF-07321332 MTBE solvate, compound I′) in isopropyl acetate, heptane addition time, seed size and seed amount had detectable impacts on final particle size distribution. Statistical analysis was carried out using Design Expert. Analysis results are summarized graphically using main effect plots and contour plots to facilitate visualization of the statistically significant factor effects and to allow their practical importance to be assessed. FIG. 1 shows the relationship between the initial concentration of API in isopropyl acetate (mL/g), the heptane addition time (in hours), the seed load (% wt compound I, Form I/wt of compound I′, MTBE solvate) and the seed size (in microns) on the final D[v, 0.5] counts in micron of particle size distributions (PSD's).

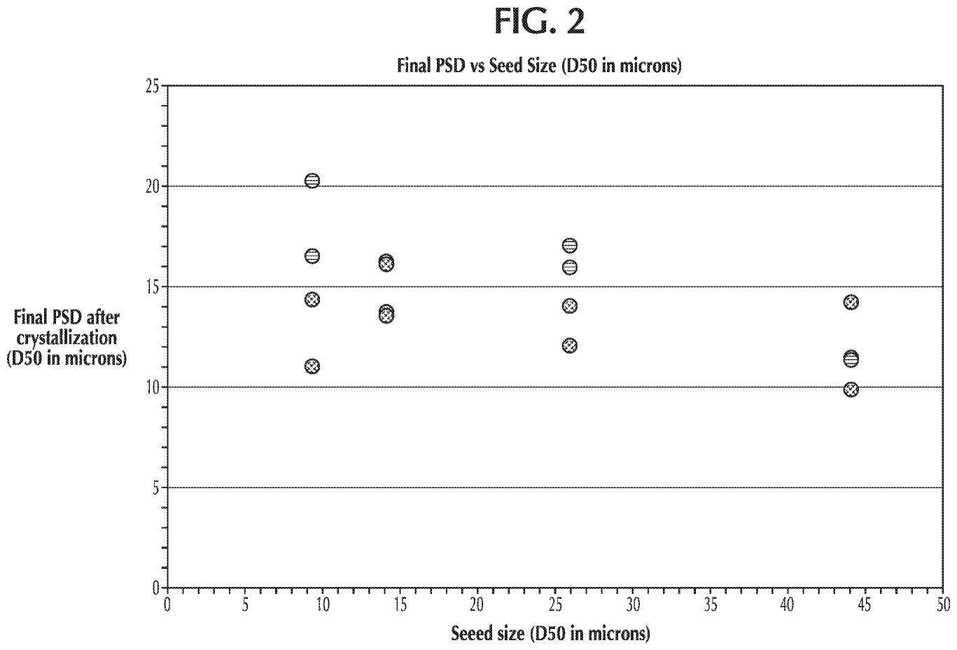

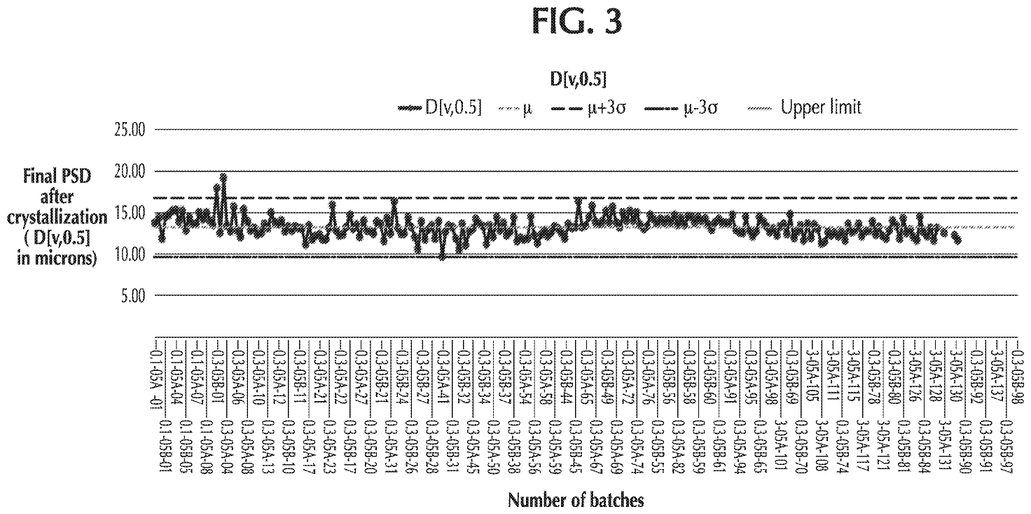

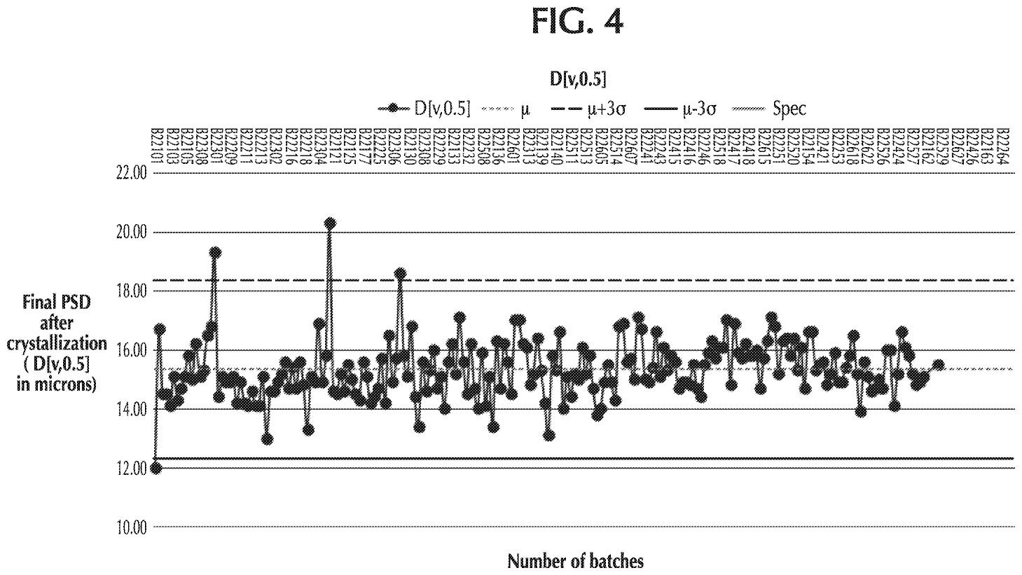

The crystallization process has been run on small to large scale in a robust and consistent manner. Furthermore, the target particle size was achieved via direct crystallization with no post crystallization milling operation necessary. The process shows size dependent growth which relies on the size of the seed material. To minimize the dependence on the seed size, the seed load, and the duration of heptane addition was adjusted. The seed load was optimized between 0.2 wt % to 1.5 wt % with a target value of 0.75 wt % throughout all scales. The duration of heptane addition was adjusted to 10 hours with a range of 6 hours to 15 hours. These changes helped achieve the desired final particle size independent to ingoing seed particle size. The process relies on primary and secondary nucleation instead of size dependent crystal growth. The data obtained from different scales shows consistent particle size delivery with minimal variations. The process wherein the seed load is about 0.75 wt % and the heptane is added over about 10 hours provides for consistent production of nirmatrelvir with the particle size distribution being well controlled. FIG. 2 shows the correlation between seed size and final particle size distribution after crystallization of an API with 0.75 wt % seed load (blue circles) and 0.5 wt % seed load (orange circles) on laboratory scale (100 mL) experiments. Batch history data shown in FIG. 3 and FIG. 4 from two different locations shows the process delivers the particle size distributions in a robust and consistent manner, in each instance showing the PSDs obtained for D[v,0.5] of more than 50 batches. Particle size distributions for the individual batches were generally found to have a D[v,0.5] in the ranges of about 12 microns to about 18 microns, about 14 microns to about 16 microns with most having a D[v,0.5] of about 15 microns.

### EXPERIMENTAL PROCEDURES

The following illustrate the synthesis of various compounds of the present invention. Additional compounds within the scope of this invention may be prepared using the methods illustrated in these Examples, either alone or in combination with techniques generally known in the art. All starting materials in these Preparations and Examples are either commercially available or can be prepared by methods known in the art or as described herein.

All reactions were carried out using continuous stirring under an atmosphere of nitrogen or argon gas unless otherwise noted. When appropriate, reaction apparatuses were dried under dynamic vacuum using a heat gun, and anhydrous solvents (Sure-Seal™ products from Aldrich Chemical Company, Milwaukee, Wisconsin or DriSolv™ products from EMD Chemicals, Gibbstown, NJ) were employed. In some cases, commercial solvents were passed through columns packed with 4 Å molecular sieves, until the following QC standards for water were attained: a) <100 ppm for dichloromethane, toluene, N,N-dimethylformamide, and tetrahydrofuran; b) <180 ppm for methanol, ethanol, 1,4-dioxane, and diisopropylamine. For very sensitive reactions, solvents were further treated with metallic sodium, calcium hydride, or molecular sieves, and distilled just prior to use. Other commercial solvents and reagents were used without further purification. For syntheses referencing procedures in other Examples or Methods, reaction conditions (reaction time and temperature) may vary. Products were generally dried under vacuum before being carried on to further reactions or.

Reaction progress can be monitored using thin-layer chromatography (TLC), liquid chromatography-mass spectrometry (LCMS), high-performance liquid chromatography (HPLC), and/or gas chromatography-mass spectrometry (GCMS) analyses. TLC can be performed on pre-coated silica gel plates with a fluorescence indicator (254 nm excitation wavelength) and visualized under UV light and/or with 12, KMnO₄, CoCl₂, phosphomolybdic acid, and/or ceric ammonium molybdate stains. LCMS data can be acquired on an Agilent 1100 Series instrument with a Leap Technologies autosampler, Gemini C18 columns, acetonitrile/water gradients, and either trifluoroacetic acid, formic acid, or ammonium hydroxide modifiers. The column eluate is analyzed using a Waters ZQ mass spectrometer scanning in both positive and negative ion modes from 100 to 1200 Da. Other similar instruments can also be used. HPLC data were generally acquired on an Agilent 1100 Series instrument, using the columns indicated, acetonitrile/water gradients, and either trifluoroacetic acid or ammonium hydroxide modifiers. GCMS data are acquired using a Hewlett Packard 6890 oven with an HP 6890 injector, HP-1 column (12 m×0.2 mm×0.33 μm), and helium carrier gas. The sample can be analyzed on an HP 5973 mass selective detector scanning from 50 to 550 Da using electron ionization. Purifications are performed by medium performance liquid chromatography (MPLC) using Isco CombiFlash Companion, AnaLogix IntelliFlash 280, Biotage SP1, or Biotage Isolera One instruments and pre-packed Isco RediSep or Biotage Snap silica cartridges. Chiral purifications were performed by chiral supercritical fluid chromatography (SFC), generally using Berger or Thar instruments; columns such as ChiralPAK-AD, -AS, -IC, Chiralcel-OD, or -OJ columns; and CO₂ mixtures with methanol, ethanol, 2-propanol, or acetonitrile, alone or modified using trifluoroacetic acid or propan-2-amine. UV detection can be used to trigger fraction collection. For syntheses referencing procedures in other Examples or Methods, purifications may vary: in general, solvents and the solvent ratios used for eluents/gradients were chosen to provide appropriate Ris or retention times.

Mass spectrometry data are reported from LCMS analyses. Mass spectrometry (MS) is performed via atmospheric pressure chemical ionization (APCI), electrospray ionization (ESI), electron impact ionization (EI) or electron scatter ionization(ES) sources. Proton nuclear magnetic spectroscopy (¹H NMR) chemical shifts are given in parts per million downfield from tetramethylsilane and were recorded on 300, 400, 500, or 600 MHz Varian, Bruker, or Jeol spectrometers. Chemical shifts are expressed in parts per million (ppm, 8) referenced to the deuterated solvent residual peaks (chloroform, 7.26 ppm; CD₂HOD, 3.31 ppm; acetonitrile-d₂, 1.94 ppm; dimethyl sulfoxide-d₅, 2.50 ppm; DHO, 4.79 ppm). The peak shapes are described as follows: s, singlet; d, doublet; dd, doublet of doublet; ddd, doublet of doublet of doublet; dt, doublet of triplet; t, triplet; q, quartet; qd, quartet of doublet; quin, quintet; m, multiplet; br s, broad singlet; app, apparent. Analytical SFC data were generally acquired on a Berger analytical instrument as described above. Optical rotation data were acquired on a PerkinElmer model 343 polarimeter using a 1 dm cell. Microanalyses were performed by Quantitative Technologies Inc. and were within 0.4% of the calculated values.

Unless otherwise noted, chemical reactions were performed at room temperature (about 23 degrees Celsius). Unless noted otherwise, all reactants were obtained commercially and used without further purification, or were prepared using methods known in the literature.

The terms “concentrated”, “evaporated”, and “concentrated in vacuo” refer to the removal of solvent at reduced pressure on a rotary evaporator with a bath temperature less than 60° C., or at a temperature as specified. The abbreviations “min” and “h” stand for “minutes” and “hours,” respectively. The term “TLC” refers to thin-layer chromatography, “room temperature or ambient temperature” means a temperature between 18 to 25° C., “GCMS” refers to gas chromatography-mass spectrometry, “LCMS” refers to liquid chromatography-mass spectrometry, “UPLC” refers to ultra-performance liquid chromatography, “HPLC” refers to high-performance liquid chromatography, and “SFC” refers to supercritical fluid chromatography. Other abbreviations used include “° C.” is degrees Celsius; “CO₂” is carbon dioxide; “eq.” or “equiv.” is equivalents; “DMSO-d₆” is hexadeutero dimethylsulfoxide; “g” is gram; “HCl” is hydrogen chloride; “HOPO” is 2-Hydroxypyridin-N-oxide; “HRMS” is high resolution mass spectroscopy; “Hz” is hertz; “iPrOAc” is isopropyl acetate; “K” is Kelvin; “kg” is kilogram; “L” is liter; “M” is mole or molar; “mbar” is millibar; “MEK” is methyl ethyl ketone; “MeOH” is methanol; “MHz” is megahertz; “mg” is milligrams; “μg” is micrograms; “min” is minutes; “mL” is milliliter; “μL” is microliter; “mm” is millimeter; “mmol” is millimole; “μmol” is micromole; “MTBE” is methyl tert-butyl ether; “NaCl” is sodium chloride; “NaHCO₃” is sodium bicarbonate; “Na₂SO₄” is sodium sulfate; “PXRD” is powder x-ray diffraction; and “THF” is tetrahydrofuran.

Hydrogenation may be performed in a Parr shaker under pressurized hydrogen gas, or in a Thales-nano H-Cube flow hydrogenation apparatus at full hydrogen and a flow rate between 1-2 mL/min at specified temperature or as otherwise specified.

HPLC, UPLC, LCMS, GCMS, and SFC retention times are measured using the methods noted in the procedures.

The optical rotation of an enantiomer can be measured using a polarimeter.

According to its observed rotation data (or its specific rotation data), an enantiomer with a clockwise rotation was designated as the (+)-enantiomer and an enantiomer with a counter-clockwise rotation was designated as the (−)-enantiomer. Racemic compounds are indicated either by the absence of drawn or described stereochemistry, or by the presence of (+/−) adjacent to the structure; in this latter case, the indicated stereochemistry represents just one of the two enantiomers that make up the racemic mixture.

The compounds and intermediates described below were named using the naming convention provided with ACD/ChemSketch 2019.1.1, File Version C05H41, Build 110712 (Advanced Chemistry Development, Inc., Toronto, Ontario, Canada) or using the structure naming function in ChemDraw 18.0 (PerkinElmer® Informatics; perkinelmer.com). The naming convention provided with ACD/ChemSketch 2019.1.1 or ChemDraw 18.0 is well known by those skilled in the art and it is believed that the naming convention provided with ACD/ChemSketch 2019.1.1 generally comports with the IUPAC (International Union for Pure and Applied Chemistry) recommendations on Nomenclature of Organic Chemistry and the CAS Index rules.

### General Method for Solid Form Characterization

Solid Form Data was obtained for certain of the starting materials, intermediates and products using the Powder X-Ray Diffraction Method below.

### Powder X-Ray Diffraction

The powder X-ray diffraction analysis was conducted using a Bruker AXS D4 Endeavor diffractometer equipped with a Cu radiation source. The divergence slit was set at 0.6 mm while the secondary optics used variable slits. Diffracted radiation was detected by a PSD-Lynx Eye detector. The X-ray tube voltage and amperage were set to 40 kV and 40 mA respectively. Data was collected in the Theta-2Theta goniometer at the Cu wavelength from 3.0 to 40.0 degrees 2-Theta using a step size of 0.020 degrees and a step time of 0.3 second. Samples were prepared by placing them in a silicon low background sample holder and rotated during collection.

The powder X-ray diffraction analysis was conducted using a Bruker AXS D8 Advance diffractometer equipped with a Cu radiation source. Diffracted radiation was detected by a LYNXEYE_EX detector with motorized slits. Both primary and secondary equipped with 2.5 soller slits. The X-ray tube voltage and amperage were set at 40 kV and 40 mA respectively. Data was collected in the Theta-Theta goniometer in a locked couple scan at Cu K-alpha (average) wavelength from 3.0 to 40.0 degrees 2-Theta with an increment of 0.02 degrees, using a scan speed of 0.5 seconds per step. Samples were prepared by placement in a silicon low background sample holder.

Data were collected with both instruments using Bruker DIFFRAC Plus software and analysis was performed by EVA diffract plus software. The PXRD data file was not processed prior to peak searching. Using the peak search algorithm in the EVA software, peaks selected with a threshold value of 1 were used to make preliminary peak assignments. To ensure validity, adjustments were manually made; the output of automated assignments was visually checked, and peak positions were adjusted to the peak maximum. Peaks with relative intensity of >3% were generally chosen. Typically, the peaks which were not resolved or were consistent with noise were not selected. A typical error associated with the peak position from PXRD stated in USP up to +/−0.2° 2-Theta (USP-941). Unless stated otherwise the variance for each peak reported in the PXRD peak tables is +/−0.2° 2-theta.

### Preparation of Starting Materials

### Preparation of Methyl (1R,2S,5S)-6,6-dimethyl-3-azabicyclo[3.1.0]hexane-2-carboxylate, Hydrochloride salt; Compound VII

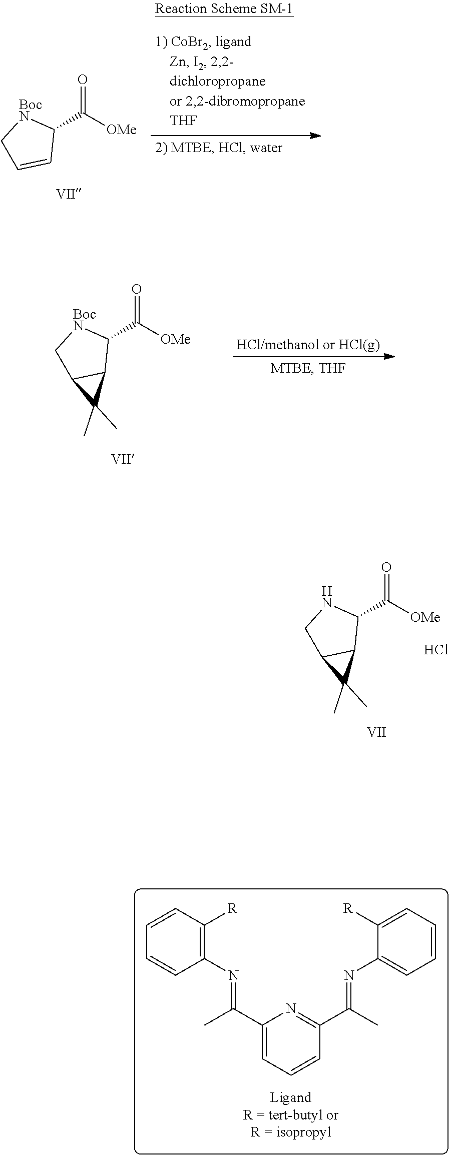

The title compound is prepared as depicted above in Reaction Scheme SM-1 according to the following procedure.

CoBr₂ (0.05-0.15 equiv), (1E,1′E)-1,1′-(pyridine-2,6-diyl)bis(N-(2-(tert-butyl)phenyl)ethan-1-imine) or (1E,1′E)-1,1′-(pyridine-2,6-diyl)bis(N-(2-isopropylphenyl)ethan-1-imine) (0.05-0.15 equiv, i.e. the ligand), and tetrahydrofuran (10 vol) were charged to a reactor. Zn (2.25-2.5 equiv.) was charged. I₂ (0.25 equiv.) in tetrahydrofuran (1-2 vol) was charged. A purple solution was obtained. 1-(tert-butyl) 2-methyl (S)-2,5-dihydro-1H-pyrrole-1,2-dicarboxylate (1 equiv., compound VII″) was charged as a neat oil. 2,2-dichloropropane or 2,2-dibromopropane (1.5-2.0 equiv.) in tetrahydrofuran (1-3 vol) was slowly added and stirred until reaction completion (formation of compound VII′). The reaction mixture was filtered through Celite. Methyl tert-butyl ether (MTBE), HCl, and water were used during this operation. The organic phase was washed with HCl, water and dried with magnesium sulfate or sodium sulfate. The solution was concentrated to 1-2 volume. The resulting solution was treated with HCl (3 equiv.) in methanol or with tetrahydrofuran and HCl gas (3 equiv.). Upon reaction completion, methyl (1R,2S,5S)-6,6-dimethyl-3-azabicyclo[3.1.0]hexane-2-carboxylate, hydrochloride salt was isolated from MTBE and THE as a solid. Typical yields are 50-80%. For example, 3.00 g of 1-(tert-butyl) 2-methyl (S)-2,5-dihydro-1H-pyrrole-1,2-dicarboxylate was converted to 1.98 g of methyl (1R,2S,5S)-6,6-dimethyl-3-azabicyclo[3.1.0]hexane-2-carboxylate, hydrochloride salt (80% yield). Product characterization data was consistent with that reported previously: Oruganti, S. et al. Tetrahedron, 2017, 73, 4285.

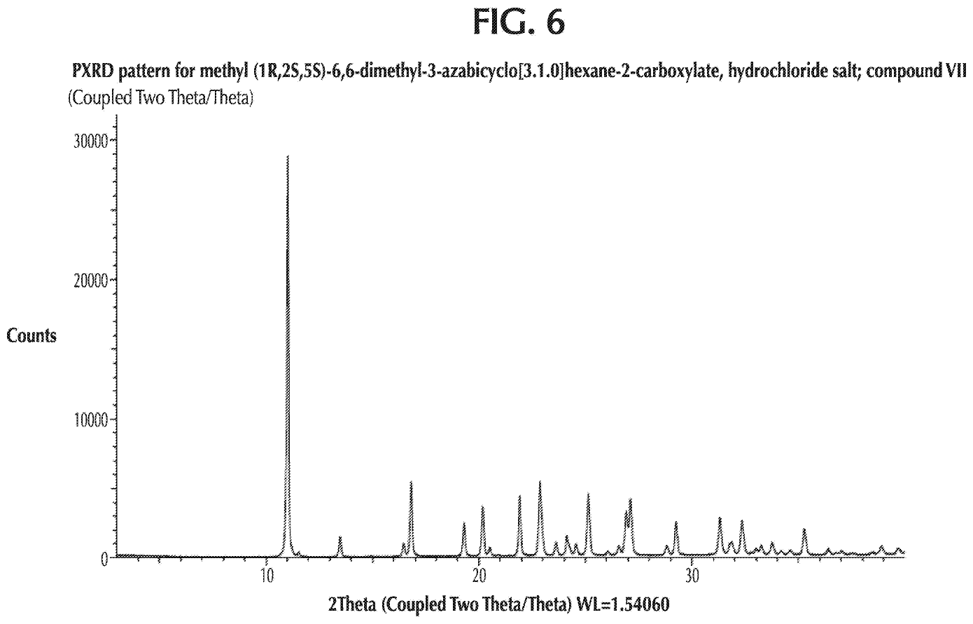

The PXRD for the product methyl (1R,2S,5S)-6,6-dimethyl-3-azabicyclo[3.1.0]hexane-2-carboxylate, hydrochloride salt; compound VII is provided in FIG. 6.

### Preparation of Methyl(S)-2-amino-3-((S)-2-oxopyrrolidin-3-yl)propanoate Para-Toluene Sulfonic Acid Salt

The title compound is prepared as depicted in Reaction Scheme SM-2 and as described below.

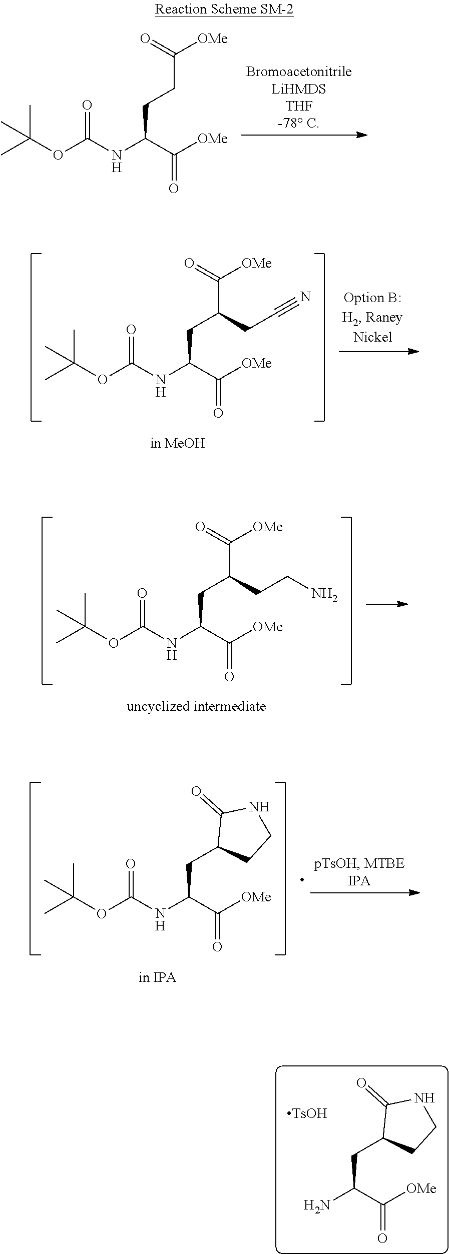

To a reactor was charged dimethyl (tert-butoxycarbonyl)-L-glutamate (50.2 g, 1.00 equiv) and Tetrahydrofuran (753 mL, 15 mL/g). The solution was cooled to −78° C. in a dry ice/acetone bath. A solution of Lithium Bis (trimethylsilyl) amide (1 M) in THF (371 mL 2.1 equiv) was precooled and added via cannula. A solution of Bromoacetonitrile (13.5 mL, 1.07 eq) in THF (70 mL, 1.4 mL/g) was precooled and charged to the reactor. The mixture was stirred for at least 45 min after addition was complete, maintaining temperature below −70° C. To the reaction mixture, a solution of Methanol (20 mL, 0.4 mL/g) in Tetrahydrofuran (25 mL, 0.5 mL/g) was charged. To the reaction mixture was charged Acetic acid (40.5 mL, 4 equiv) in tetrahydrofuran (100 mL, 2 mL/g) via addition funnel. The mixture was warmed to −20° C. over 30 minutes. To the mixture, charge Sodium chloride (12 mass %) in water (251 mL, 5 mL/g). The layers were separated. The organic layer was concentrated to 2-3 mL/g. To the residue, Toluene (1000 L, 20 mL/g) was charged. The mixture was concentrated to ˜20 mL/g, then a constant volume distillation was performed, maintaining 20 mL/g. To the solution was charged

Diatomaceous earth (10 g, 0.2 g/g) and the slurry stirred for 2 h. The slurry was filtered over a bed of Diatomaceous earth (10 g, 0.2 g/g). The filter cake was washed with toluene (1 mL/g). The filtrate was concentrated to ˜2-3 mL/g. Methanol (250 mL, 5 mL/g) was charged. A constant volume distillation was performed, displacing with Methanol (650 mL, 13 mL/g). The resulting dimethyl (2S,4R)-2-((tert-butoxycarbonyl)amino)-4-(cyanomethyl)pentanedioate is carried forward as a pale amber solution (44.5 g in solution, 80% in situ yield)

To a reactor was charged Raney nickel 2400 (6.05 g, 100 wt %). A solution of dimethyl (2S,4R)-2-((tert-butoxycarbonyl)amino)-4-(cyanomethyl)pentanedioate in methanol (6 g input, 40.83 g solution) was charged, followed by additional methanol (120 mL, 20 mL/g quantity) and ammonia (7N in MeOH, 2.7 mL, 1.0 eq) The reactor was purged with N₂ three times, then H₂ three times. The reactor was pressurized to 5 bar and heated to 24° C. and stirred for 24 h. The reactor was purged and sampled to confirm reaction was complete (i.e. that the uncyclized intermediate dimethyl (2S,4S)-2-(2-aminoethyl)-4-((tert-butoxycarbonyl) amino) pentanedioate was converted to the cyclized intermediate methyl (S)-2-((tert-butoxycarbonyl)amino)-3-((S)-2-oxopyrrolidin-3-yl)propanoate). The reaction was filtered, and catalyst washed with twice with methanol (5 mL/g, 30 mL). To prepare for the next step, the filtrate was concentrated to ˜2-3 mL/g in vacuo. Isopropanol (20 mL/g, 125 mL) was charged and concentrated to remove residual MeOH and water to target 7 mL/g total volume (6 mL/g IPA). To the solution was charged para-toluene sulfonic acid (pTsOH) monohydrate (1.5 equiv). The mixture was diluted with Isopropanol (20 mL/g) and the solution re-concentrated to remove water. To the reactor was charged MTBE (4 mL/g). The slurry was warmed to 50° C. and held overnight. The mixture was cooled to 10° C. over 2 h. The mixture was held for 2 h, then filtered. The filter cake was washed with isopropanol and product dried in a vacuum oven at 45-50° C. 5.86 g of methyl (S)-2-amino-3-((S)-2-oxopyrrolidin-3-yl)propanoate para-toluene sulfonic acid salt is isolated as a white, crystalline solid (86% yield over 2 steps).

¹H NMR (600 MHz, DMSO-d₆, 298K): δ 8.54 (s, 2H), 7.98 (s, 1H), 7.95 (s, 1H), 7.65 (s, 1H), 7.49 (d, J=8.0 Hz, 2H), 7.12 (d, J=8.0 Hz, 2H), 4.32 (m, 1H), 4.21 (m, 1H), 3.75 (s, 3H), 3.19 (m, 2H), 2.56 (m, 1H), 2.55 (m, 1H), 2.29 (s, 3H), 2.26 (m, 1H), 2.01 (m, 1H), 1.89 (m, 1H), 1.66 (m, 1H). ¹³C NMR (150 MHz, DMSO-d₆, 298K): δ 178.2, 169.7, 145.5, 137.6, 128.0, 125.4, 52.8, 51.2, 39.8, 38.3, 31.6, 27.6, 20.7. HRMS: (ESI⁺) Calcd for C₈H₁₅N₂O₃⁺: 187.1077, Found: 187.1077

### Alternate Preparation of Methyl (S)-2-amino-3-((S)-2-oxopyrrolidin-3-yl)propanoate Para-Toluene Sulfonic Acid Salt

The title compound is prepared as depicted in Reaction Scheme SM-3 and as described below.

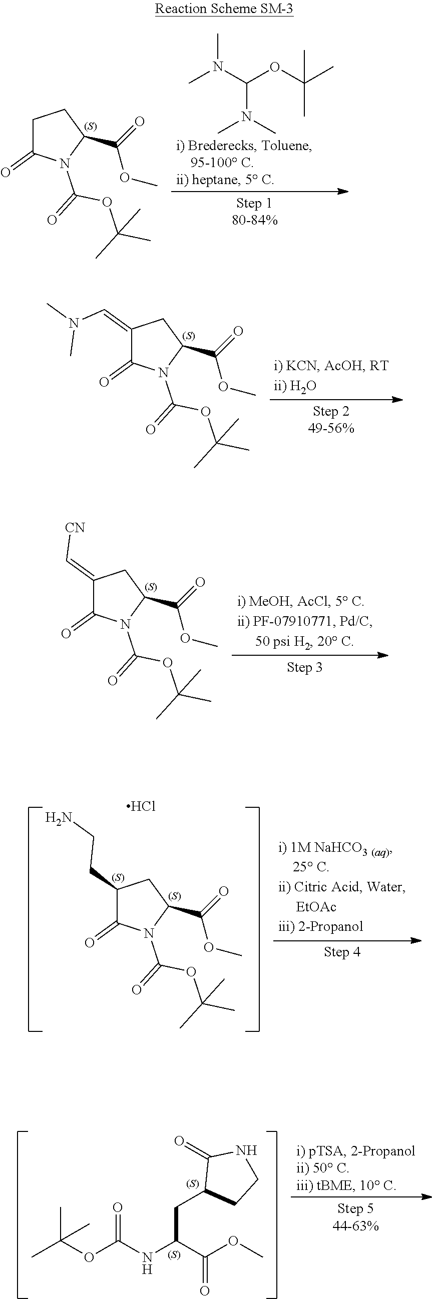

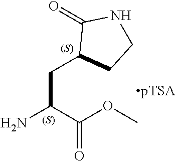

### Step 1: Preparation of 1-(tert-butyl) 2-methyl (S,Z)-4-((dimethylamino)methylene)-5-oxopyrrolidine-1,2-dicarboxylate

1-(tert-butyl) 2-methyl (S)-5-oxopyrrolidine-1,2-dicarboxylate (10.00 g) was added to toluene (60 mL) at 20° C. with stirring to provide a clear colorless solution. To this solution was added tert-Butoxy bis(dimethylamino) methane (Brederecks reagent) (8.60 g, 10.2 mL, 1.2eq.) in a single portion and the resulting reaction mixture was stirred at temperature 95-100° C. for 16 hours. Reaction was checked for completion by UPLC (2.7% staring material remained) and the temperature was then adjusted to 80° C. 40 mL of n-heptane was added to provide a yellow solution and the mixture was cooled to 60° C. at which point product seed can be added if available. The mixture was cooled to 5° C. over 2 hours and the resulting slurry was stirred at 5° C. for 2 hours. The mixture was filtered and the resulting filter cake was washed with toluene (10 mL). The resulting solid was dried under vacuum overnight at 40° C. to provide 1-(tert-butyl) 2-methyl (S,Z)-4-((dimethylamino) methylene)-5-oxopyrrolidine-1,2-dicarboxylate as a white to off-white solid (10.3 g, 84% yield). ¹H NMR (400 MHz, Dimethylsulfoxide-d6) δ 6.97 (t, 1H), 4.50 (dd, 1H), 3.69 (s, 3H), 3.22 (m, 1H), 2.99 (s, 6H), 2.81 (m, 1H), 1.39 (s, 9H). >97% purity by ¹H NMR

### Step 2: Preparation of 1-(tert-butyl) 2-methyl (S,E)-4-(cyanomethylene)-5-oxopyrrolidine-1,2-dicarboxylate

To a 1-neck 250 mL round bottom flask equipped with a toxic gas scrubber and under an inert atmosphere was added 1-(tert-butyl) 2-methyl (S,Z)-4-((dimethylamino) methylene)-5-oxopyrrolidine-1,2-dicarboxylate (5.00 g, 1.00 eq.) and acetic acid (30.0 mL). The mixture was stirred at 20° C. to form a solution and to it was added potassium cyanide (1.20 g, 1.10 eq). The reaction mixture was stirred for 24 hours at 20° C. resulting in a dark yellow solution. To the dark yellow solution was slowly add water (210 mL, 7 volumes) over 25 min to precipitate the product. The mixture was then stirred for 5 minutes before filtration on a sinter funnel. The resulting white solid was washed with water (3×30 mL) and dried on the sinter funnel under vacuum with air flow. The resulting 1-(tert-butyl) 2-methyl (S,E)-4-(cyanomethylene)-5-oxopyrrolidine-1,2-dicarboxylate was obtained as a white solid (2.47 g, 51% yield). ¹H NMR (396 MHz, Chloroform-d) δ 6.34 (t, J=3.0 Hz, 1H), 4.74 (dd, J=9.7, 3.0 Hz, 1H), 3.81 (s, 3H), 3.29 (qd, J=9.9, 3.5 Hz, 1H), 3.01 (dt, J=19.6, 2.7 Hz, 1H), 1.52 (s, 9H). >95% purity by ¹H NMR. This material was used in the next step without further purification.

### Step 3: Preparation of 1-(tert-butyl) 2-methyl (2S,4S)-4-(2-aminoethyl)-5-oxopyrrolidine-1,2-dicarboxylate Hydrochloride Salt (as a Solution in Methanol)

Methanol (200 ml, 20 ml/g) is stirred in a reactor at 5° C. and to it is added acetyl chloride (2.79mL, 1.1 eq.) which is added dropwise to control the resulting exotherm to generate anhydrous HCl (and 1.1 eq methyl acetate by-product). The solution is then warmed to 20° C. 1-(tert-butyl) 2-methyl (S,E)-4-(cyanomethylene)-5-oxopyrrolidine-1,2-dicarboxylate (10.0 g, 1.0 eq., limiting reagent) is added to the methanolic HCl solution in a single portion as a solid to give a clear colourless solution. To this solution was charged 5% Pd/C type A503023-5 (2.0 g). The reactor was purged with nitrogen three times, then with hydrogen three times and then was pressurised to 50 psi with hydrogen and the mixture was stirred at 600 rpm for 16 hours. The reactor was purged and sampled to confirm reaction completion. The reaction was incomplete (>3% residual nitrile intermediate) therefore was hydrogenated for an additional 16 hours to reduce the nitrile intermediate content from 20% to 3%. The reaction mixture was filtered through Arbocel to remove the catalyst and the filter washed with methanol (2×10 mL, 2×1 mL/g). The resulting methanol filtrate containing 1-(tert-butyl) 2-methyl (2S,4S)-4-(2-aminoethyl)-5-oxopyrrolidine-1,2-dicarboxylate hydrochloride salt is used as is in the next step.

### Alternate Step 3: Preparation of 1-(tert-butyl) 2-methyl (2S,4S)-4-(2-aminoethyl)-5-oxopyrrolidine-1,2-dicarboxylate Hydrochloride Salt (as a Solution in Methanol/Isopropanol)

Methanol (200 ml) is stirred at 5° C. and acetyl chloride (2.79 mL, 1.1 eq.) is added dropwise to control the resulting exotherm to generate anhydrous HCl (and 1.1 eq methyl acetate by-product). In a separate hydrogenation vessel 2-propanol (50 mL) is stirred at 20° C. and to it is added 1-(tert-butyl) 2-methyl (S,E)-4-(cyanomethylene)-5-oxopyrrolidine-1,2-dicarboxylate (10.0 g, 1.0 eq., limiting reagent) resulting in a slurry. 5% Pd/C type A503023-5 (2.0 g) is charged to the slurry as a solid. The reactor was purged with nitrogen three times, then hydrogen three times then was pressurized to 50 psi with hydrogen and stirred at 600 rpm for 2 hours. The reactor was purged with nitrogen and then the methanolic HCl solution was added. The reactor was purged with nitrogen three times, then hydrogen three times then was pressurized to 50 psi with hydrogen and stirred at 600 rpm for 3 days. The reactor was purged and sampled to confirm reaction completion (>3% residual nitrile intermediate) then was filtered through Arbocel to remove the catalyst and the filter washed with methanol (2×10 mL). The resulting methanol/2-propanol filtrate containing 1-(tert-butyl) 2-methyl (2S,4S)-4-(2-aminoethyl)-5-oxopyrrolidine-1,2-dicarboxylate hydrochloride salt is used as is in the next step.

### Step 4: Preparation of Methyl (S)-2-((tert-butoxycarbonyl)amino)-3-((S)-2-oxopyrrolidin-3-yl)propanoate (as a Solution in Isopropanol)

The methanol solution of 1-(tert-butyl) 2-methyl (2S,4S)-4-(2-aminoethyl)-5-oxopyrrolidine-1,2-dicarboxylate hydrochloride salt from the preceding step 3 is stirred at 25° C. and 1M aquesous NaHCO₃ (89.2 mL, 2.5 eq.) is slowly added to it to control the rate of resulting CO₂ off-gas. The resulting fine suspension is stirred at 25° C. for 18 hours or until reaction is confirmed complete by liquid chromatography (not more than 0.5% area residual starting material remaining). The reaction is then quenched by portion wise addition of citric acid (4.11 g, 0.60 eq.) in water (30 mL) and tested to confirm that the pH is between 5-7 by pH indicator paper. The mixture is then concentrated in vacuo (90-100 mbar) to a volume of approximately 100-120 mL. To this is added ethyl acetate (100 mL), the mixture is stirred at 25° C. for 10 minutes and then the organic and aqueous layers are separated. Ethyl acetate (100 mL) is added to the aqueous layer and stirred for 10 minutes at 25° C. and then the layers are separated. The organic layers are combined and concentrated in vacuo (200 mbar) to a final volume of approximately 30 mL. To this is added 2-Propanol (200 mL) and the solution is concentrated in vacuo (100 mbar) to a final volume of approximately 70 mL. The isopropanol solution of methyl (S)-2-((tert-butoxycarbonyl)amino)-3-((S)-2-oxopyrrolidin-3-yl)propanoate is checked by Karl Fischer analysis to confirm the water content is less than 1% by weight and is then used as is in the next step.

### Step 5: Preparation of Methyl (S)-2-amino-3-((S)-2-oxopyrrolidin-3-yl)propanoate Para-Toluene Sulfonic Acid Salt

The isopropanol solution of methyl (S)-2-((tert-butoxycarbonyl)amino)-3-((S)-2-oxopyrrolidin-3-yl)propanoate from step 4 is stirred at 20° C. In a separate vessel add isopropanol (200 mL) and p-Toluenesulfonic acid monohydrate (10.33 g, 1.5 eq.). Concentrate the p-TSA solution in vacuo (90-100 mbar) to a final volume of approximately 70 mL to remove water. The concentrated p-TSA solution is then added to the methyl (S)-2-((tert-butoxycarbonyl)amino)-3-((S)-2-oxopyrrolidin-3-yl)propanoate solution followed by a line rinse of 2-Propanol (20 mL). The solution is distilled under vacuum (90-100 mbar) with jacket temperature 40° C. to a final volume of approximately 70 mL (7 mL/g) to remove further water. The solution is sampled for Karl-Fischer analysis (not more than 1 wt % water). If >1% wt water add additional 2-Propanol as required and repeat the vacuum distillation in step 5 until the target water content is achieved. The reaction mixture is heated to 50° C. at atmospheric pressure and stirred for 12-18 hours until reaction completion achieved (no starting material observed by liquid chromatography). The desired product crystallizes during this hold. Upon reaction completion tert-butylmethyl ether (50 mL) is added in a single portion. The resulting slurry is cooled from 50° C. to 10° C. over 2 hours. Stir the slurry at 10° C. for 1 hour and then filter under vacuum. Rinse the crystallization vessel with tert-butylmethyl ether (40 mL) and transfer to the filter as a cake wash. Pull the product cake dry under vacuum to de-liquor and then dry the product under vacuum at 40° C. The desired methyl (S)-2-amino-3-((S)-2-oxopyrrolidin-3-yl)propanoate para-toluene sulfonic acid salt is isolated as a white crystalline solid.

The Steps 3-5 yield from the process above using the MeOH/AcCI hydrogenation is 44% (20% overall yield for steps 1-5) with the product having 98.7% Achiral purity, 0.58% RRT 0.194, 0.28% RRT 1.783; 1% diastereomer 1, 0.68% diastereomer 2, approx. 1% enantiomer and 98.7% wt by Q-NMR. The Steps 3-5 yield from the process using isopropanol 2 stage hydrogenation is 63% (30% overall yield for steps 1-5) with the product having 98.7% Achiral purity, 0.65% RRT 0.194, 0.24% RRT 1.784; 1% diastereomer 1, 0.40% diastereomer 2, approx. 1% enantiomer and 99.2% wt by Q-NMR.

### Preparation of [(1R)-7,7-Dimethyl-2-oxo-norbornan-1-yl] Methane Sulfonic Acid Methyl (2S)-2-amino-3-[(3S)-2-oxopyrrolidin-3-yl]propanoate

The title compound is prepared as depicted in Reaction Scheme SM-4 and as described below.

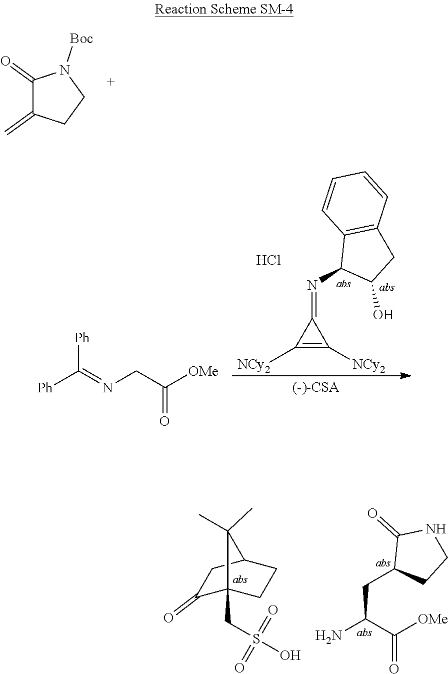

A 2M aqueous solution of NaOH (4.8 mL, 4.8 mmol) was added to a mixture of (1S,2S)-1-[(2,3-Dimethylcycloprop-2-en-1-ylidene)amino]indan-2-ol hydrochloride (557 mg, 0.96 mmol) in MTBE (6 mL) at 20° C., and stirred for 10 min. The organic layer was collected, and the aqueous layer was rinsed with MTBE (4 mL). The combined organic fractions were filtered over Na₂SO₄ and the filtrate was added directly to a mixture of methyl 2-(benzhydrylideneamino) acetate (97.0%, 5.00 g, 19.1 mmol) and tert-butyl 3-methylene-2-oxo-pyrrolidine-1-carboxylate (4.15 g, 21.1 mmol) in MTBE (10 mL). The mixture was stirred at 35° C. for 3.5 h, then (−)-CSA (9.10 g, 38.4 mmol) was added in one portion at 35° C. The mixture was heated to 65° C. for 18 h and then diluted with acetone (70 mL). The mixture was refluxed for 20 min and diluted with MTBE (130 mL). The mixture was refluxed for 4 h, then cooled to 20° C. and filtered. The solid was washed with a 2:1 mixture of MTBE and acetone (3×30 mL). The solid was collected and dried under high vacuum to afford title compound [(1R)-7,7-Dimethyl-2-oxo-norbornan-1-yl]methanesulfonic acid methyl (2S)-2-amino-3-[(3S)-2-oxopyrrolidin-3-yl]propanoate; (5.79 g, 69%, ≥25:1 dr) as a solid. MS (ESI) [M+H−CSA]⁺187.1. ¹H NMR (500 MHz, Methanol-d4) δ 4.24 (dd, J=9.7, 3.6 Hz, 1H), 3.85 (s, 3H), 3.42-3.35 (m, 2H), 3.33-3.26 (m, 1H), 2.85-2.77 (m, 1H), 2.76 (d, J=14.8 Hz, 1H), 2.70-2.61 (m, 1H), 2.47-2.37 (m, 1H), 2.38-2.29 (m, 1H), 2.23 (ddd, J=15.1, 4.8, 3.7 Hz, 1H), 2.08-1.98 (m, 3H), 1.93-1.81 (m, 2H), 1.61 (ddd, J=13.7, 9.3, 4.3 Hz, 1H), 1.41 (ddd, J=11.9, 9.5, 4.2 Hz, 1H), 1.13 (s, 3H), 0.86 (s, 3H). Note: Four exchangeable protons not visible. ¹³C NMR (126 MHz, Methanol-d₄) δ 218.28, 181.61, 170.69, 59.57, 53.81, 53.79, 48.17, 44.05, 43.61, 42.04, 41.65, 32.96, 29.50, 27.79, 25.73, 20.43, 20.12.

### Preparation of tert-butyl(S)-3-((S)-2-((diphenylmethylene)amino)-3-methoxy-3-oxopropyl)-2-oxopyrrolidine-1-carboxylate

The title compound is prepared as depicted in Reaction Scheme SM-5 and described below.

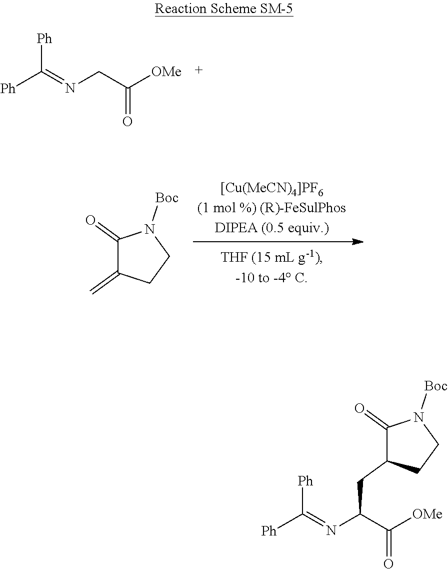 [SMILES: COC(=O)CN=C(c1ccccc1)c1ccccc1 ; C=C1CCN(C(=O)OC(C)(C)C)C1=O ; COC(=O)[C@H](C[C@@H]1CCN(C(=O)OC(C)(C)C)C1=O)N=C(c1ccccc1)c1ccccc1]

Note that all solvents used in this procedure were purchased as anhydrous grade and were degassed by bubbling with nitrogen for around 30 minutes. The reaction was performed inside an N2-filled glovebox. A solution of [Cu (MeCN)₄]PF₆ in THF (0.020 M, 25 μL, 0.5 μmol, 1 mol %) was dispensed into a 1 mL vial with a stir disc, followed by a solution of (R)-FeSulPhos in THF (0.020 M, 25 μL, 0.5 μmol, 1 mol %). This mixture was stirred at 25° C. for 23 hours, after which the THF had evaporated. A solution of diisopropylamine in THF was added (1.0 M, 25 μL, 25 μmol, 0.5 equiv.), and the vial was then cooled to −10° C. while stirring at 500 rpm. A solution of methyl 2-((diphenylmethylene)amino)acetate (12.5 mg, 49 μmol, 1.0 equiv.) and tert-butyl 3-methylene-2-oxopyrrolidine-1-carboxylate (10.7 mg, 54 μmol, 1.1 equiv.) in isopropanol (170 μL) was then added, and the vial was sealed and stirred at 500 rpm at between −10° C. and −4° C. for 24 hours. A 25 μL aliquot of the reaction mixture was then removed and diluted with 1 mL of MeCN. This sample was then analysed via chiral SFC, which showed that tert-butyl(S)-3-((S)-2-((diphenylmethylene) amino)-3-methoxy-3-oxopropyl)-2-oxopyrrolidine-1-carboxylate the desired compound was formed in 74% assay yield (rt 6.17 min, m/z+ve =451). The reference time was compared to an independently synthesised sample of tert-butyl(S)-3-((S)-2-((diphenylmethylene)amino)-3-methoxy-3-oxopropyl)-2-oxopyrrolidine-1-carboxylate to confirm the identity of the product.

SFC method: (Chiracel OX-H 250 mm×4.6 mm×5 μm (P/N: 63325), mobile phase A: CO₂, mobile phase B: isopropyl alcohol+0.2% 7N ammonia in methanol, 3 mL min⁻¹, 40° C., UV detection at 210 nm. 0-1 min 5% B, 1-9 mins 5-60% B, 9-9.5 mins 60% B, 9.5-10 mins 60-5% B.

Using the procedure described above the resulting tert-butyl (S)-3-((S)-2-((diphenylmethylene)amino)-3-methoxy-3-oxopropyl)-2-oxopyrrolidine-1-carboxylate can be converted to [(1R)-7,7-Dimethyl-2-oxo-norbornan-1-yl]methanesulfonic acid methyl (2S)-2-amino-3-[(3S)-2-oxopyrrolidin-3-yl]propanoate

### Preparation of (S)-3,3-dimethyl-2-(2,2,2-trifluoroacetamido)butanoic Acid, Compound V

The title compound is prepared as depicted in Reaction Scheme SM-6 and as described below.

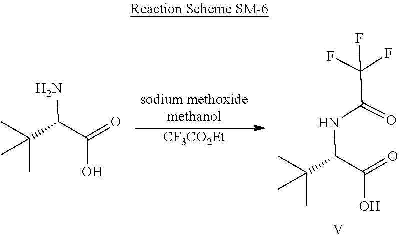

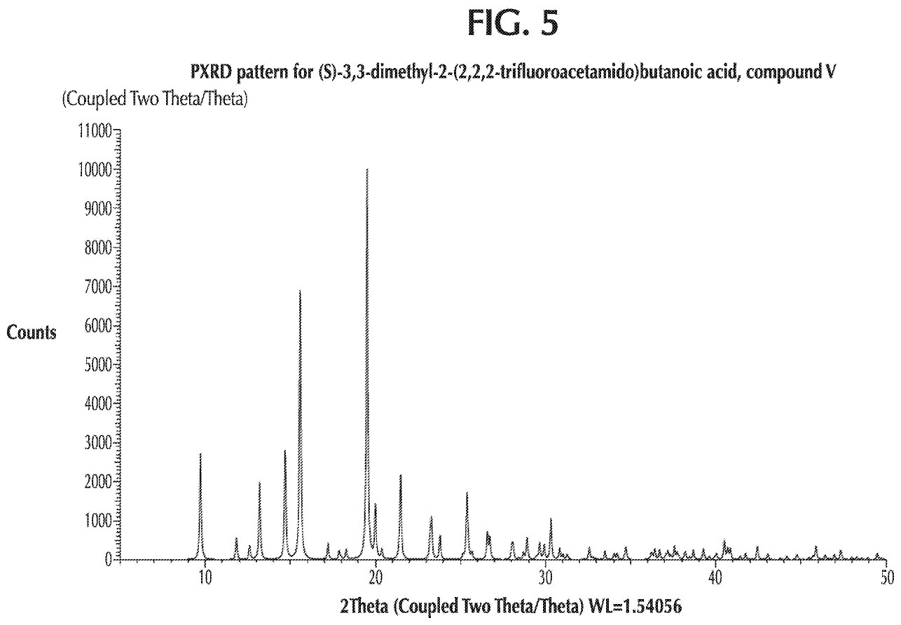

A solution of (S)-2-amino-3,3-dimethylbutanoic acid was prepared in methanol and sodium methoxide was added. Ethyl trifluoroacetate was added and the mixture stirred until the reaction was complete. The reaction was diluted with ethyl acetate and washed with brine. Heptane was added to the organic layer containing(S)-3,3-dimethyl-2-(2,2,2-trifluoroacetamido)butanoic acid and the solution concentrated to 10 mL/g at 50° C. This was repeated three times to remove as much ethyl acetate as possible. After the 2^{nd} distillation, seed crystals of (S)-3,3-dimethyl-2-(2,2,2-trifluoroacetamido)butanoic acid could be introduced. After the final distillation, the mixture was cooled to 20° C. and granulated, then filtered and washed with heptane. The solid was dried at 40° C. Crystal data was obtained and is provided in the table below and the PXRD pattern for this material is provided as FIG. 5.

TABLE
Crystal data and structure refinement for (S)-3,3-dimethyl-
2-(2,2,2-trifluoroacetamido)butanoic acid, compound V:

| Identification code | E178 | |
|---|---|---|
| Empirical formula | C8 H12 F3 N O3 | |
| Formula weight | 227.19 | |
| Temperature | 173(2) K | |
| Wavelength | 1.54178 Å | |
| Crystal system | Tetragonal | |
| Space group | P4₁2₁2 | |
| Unit cell dimensions | a = 9.9168(6) Å | α = 90°. |
| | b = 9.9168(6) Å | β = 90°. |
| | c = 22.721(2) Å | γ = 90°. |

| | |
|---|---|
| Volume | 2234.5(4) Å³ |
| Z | 8 |
| Density (calculated) | 1.351 Mg/m³ |
| Absorption coefficient | 1.184 mm⁻¹ |
| F(000) | 944 |
| Crystal size | 0.200 × 0.170 × 0.080 mm³ |
| Theta range for data collection | 4.866 to 70.114°. |
| Index ranges | −11 <= h <= 10, −12 <= k <= |
| | 12, −27 <= l <= 27 |
| Reflections collected | 48160 |
| Independent reflections | 2122 [R(int) = 0.0392] |
| Completeness to theta = 67.679° | 99.8% |
| Absorption correction | Empirical |
| Refinement method | Full-matrix least-squares on F² |
| Data/restraints/parameters | 2122/9/158 |
| Goodness-of-fit on F² | 1.010 |
| Final R indices [I > 2sigma(I)] | R1 = 0.0408, wR2 = 0.1012 |
| R indices (all data) | R1 = 0.0429, wR2 = 0.1039 |
| Absolute structure parameter | 0.03(4) |
| Extinction coefficient | n/a |
| Largest diff. peak and hole | 0.280 and −0.215 e · Å⁻³ |

FIG. 5 provides the PXRD pattern for(S)-3,3-dimethyl-2-(2,2,2-trifluoroacetamido)butanoic acid which is characterized by the PXRD peaks in the following table.

TABLE
PXRD peaks for (S)-3,3-dimethyl-2-(2,2,2-
trifluoroacetamido)butanoic acid:

| Angle | Rel. |
|---|---|
| (degrees 2 THETA) | Intensity |
| 9.7 | 27 |
| 11.8 | 6 |
| 12.6 | 3 |
| 13.2 | 20 |
| 14.7 | 28 |
| 15.6 | 69 |
| 17.2 | 4 |
| 17.9 | 2 |
| 18.3 | 2 |
| 19.5 | 100 |
| 20.0 | 14 |
| 20.4 | 2 |
| 21.5 | 22 |
| 23.2 | 8 |
| 23.3 | 11 |
| 23.8 | 6 |
| 25.2 | 1 |
| 25.4 | 17 |
| 25.7 | 2 |
| 26.6 | 7 |
| 26.7 | 6 |
| 28.1 | 4 |
| 28.7 | 2 |
| 28.9 | 5 |
| 29.7 | 4 |
| 29.9 | 4 |
| 30.3 | 10 |
| 30.8 | 3 |
| 31.0 | 1 |
| 31.3 | 1 |
| 32.6 | 3 |
| 33.5 | 2 |
| 34.0 | 1 |
| 34.2 | 2 |
| 34.7 | 3 |
| 36.2 | 2 |
| 36.4 | 3 |
| 36.7 | 2 |
| 37.1 | 1 |
| 37.2 | 2 |
| 37.4 | 1 |
| 37.6 | 3 |
| 37.7 | 1 |
| 38.2 | 2 |
| 38.7 | 2 |
| 39.3 | 3 |
| 39.6 | 1 |
| 40.0 | 1 |
| 40.5 | 5 |
| 40.7 | 3 |
| 40.9 | 3 |
| 41.5 | 1 |
| 41.8 | 1 |
| 42.4 | 3 |
| 43.1 | 1 |
| 44.2 | 1 |
| 44.8 | 1 |
| 45.7 | 1 |
| 45.9 | 3 |
| 46.4 | 1 |
| 47.0 | 1 |
| 47.3 | 2 |
| 49.5 | 2 |

### EXAMPLES

### Step 1: Preparation of sodium (1R,2S,5S)-6,6-dimethyl-3-azabicyclo[3.1.0]hexane-2-carboxylate

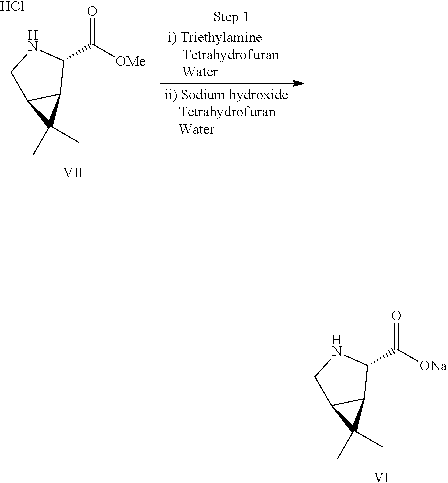

methyl (1R,2S,5S)-6,6-dimethyl-3-azabicyclo[3.1.0]hexane-2-carboxylate, hydrochloride salt (compound VII, 40 g, 195 mmol, 1.0 equivalents)), tetrahydrofuran (80 mL, 2 mL/g of compound VII) and water (40 mL, 1 mL/g of compound VII) are combined and stirred at 25° C. Triethylamine (40.7 mL, 292 mmol, 1.5 equivalents) is charged and the mixture is stirred for 30 minutes. The pH of the agitated mixture should be not less than 8.5. Agitation is stopped and the phases allowed to separate. The aqueous phase is removed to provide an organic of solution methyl (1R,2S,5S)-6,6-dimethyl-3-azabicyclo[3.1.0]hexane-2-carboxylate. In a separate vessel, sodium hydroxide (8.16 g, 204 mmol, 1.05 equivalents), tetrahydrofuran (360 mL, 9 mL/g of compound VII) and water (40 mL, 1 mL/g of compound VII) are combined and heated to 40° C. with stirring. The organic solution of methyl (1R,2S,5S)-6,6-dimethyl-3-azabicyclo[3.1.0]hexane-2-carboxylate is added to this mixture over not less than 15 minutes, and the resulting mixture is stirred for 4 hours at 40° C. A sample is analyzed for reaction completion by UPLC (target of not more than 4% methyl (1R,2S,5S)-6,6-dimethyl-3-azabicyclo[3.1.0]hexane-2-carboxylate remaining, continue stirring if the reaction is not complete). Upon completion, the mixture is cooled to 20° C. and stirred for not less than 2 hours. The solids are collected by filtration, rinsed with 96:4 THF/water (80 mL), and dried at 70° C. in a vacuum oven to provide sodium (1R,2S,5S)-6,6-dimethyl-3-azabicyclo[3.1.0]hexane-2-carboxylate, compound VI.

¹H NMR (600 MHz, CD₃OD-d₄, 298K): δ 3.37 (d, 1H), 3.31 (dd, 1H), 2.76 (dd, 1H), 1.62 (dd, 1H), 1.36 (m, 1H), 1.06 (s, 3H), 1.03 (s, 3H).

¹³C NMR (150 MHz, CD₃OD-d₄, 298K): δ 181.6, 64.3, 47.2, 37.7, 31.6, 27.1, 20.9, 14.2.

HRMS: (ESI⁺) Calcd for C₈H₁₄O₂N⁺: 156.1019, Found: 156.1020 (mass deviation +0.83 ppm)

Multiple solid forms of sodium (1R,2S,5S)-6,6-dimethyl-3-azabicyclo[3.1.0]hexane-2-carboxylate, compound VI have been isolated and characterized by PXRD.

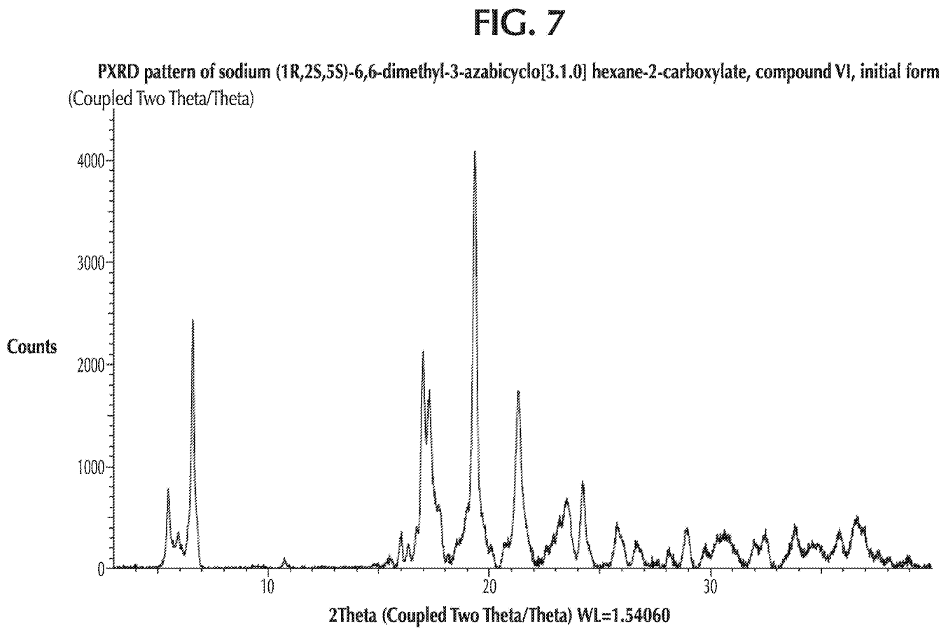

FIG. 7 provides the PXRD pattern of (1R,2S,5S)-6,6-dimethyl-3-azabicyclo[3.1.0] hexane-2-carboxylate, Initial form which is characterized by the peaks in the following table.

TABLE
PXRD peaks (degrees 2-theta) for sodium (1R,2S,5S)-6,6-dimethyl-
3-azabicyclo[3.1.0]hexane-2-carboxylate, Initial Form:

| Angle (2 theta) | Rel. Intensity |
|---|---|
| 5.5 | 19 |
| 5.9 | 9 |
| 6.6 | 59 |
| 10.7 | 2 |
| 16.0 | 9 |
| 16.3 | 6 |
| 16.7 | 10 |
| 17.0 | 52 |
| 17.3 | 42 |
| 17.7 | 15 |
| 18.5 | 7 |
| 19.0 | 15 |
| 19.3 | 100 |
| 21.3 | 45 |
| 23.2 | 12 |
| 23.5 | 17 |
| 24.2 | 20 |
| 25.8 | 11 |
| 26.0 | 8 |
| 26.7 | 4 |
| 28.1 | 3 |
| 28.9 | 6 |
| 29.8 | 4 |
| 30.3 | 8 |
| 30.8 | 8 |
| 32.0 | 3 |
| 32.5 | 5 |
| 33.8 | 7 |
| 34.6 | 4 |
| 35.8 | 6 |
| 36.6 | 8 |
| 36.9 | 7 |
| 37.6 | 3 |

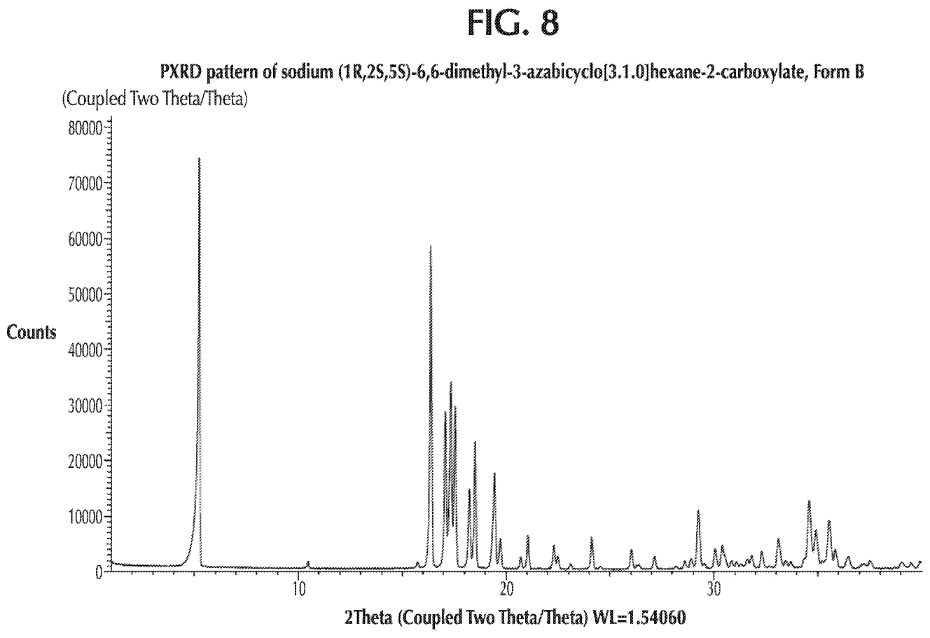

FIG. 8 provides the PXRD pattern for sodium (1R,2S,5S)-6,6-dimethyl-3-azabicyclo[3.1.0]hexane-2-carboxylate designated Form B which is characterized by the peaks in the following table.

TABLE
PXRD peaks for sodium (1R,2S,5S)-6,6-dimethyl-3-
azabicyclo[3.1.0]hexane-2-carboxylate, Form B:

| Angle | Rel. |
|---|---|
| (degrees 2 THETA) | Intensity |
| 5.2 | 100 |
| 10.5 | 2 |
| 15.7 | 1 |
| 16.4 | 80 |
| 17.1 | 38 |
| 17.3 | 46 |
| 17.5 | 40 |
| 18.2 | 20 |
| 18.5 | 35 |
| 19.4 | 23 |
| 19.7 | 7 |
| 20.7 | 3 |
| 21.1 | 8 |
| 22.3 | 6 |
| 22.5 | 3 |
| 23.1 | 1 |
| 24.1 | 8 |
| 24.5 | 1 |
| 26.0 | 4 |
| 26.3 | 1 |
| 26.4 | 1 |
| 27.1 | 3 |
| 28.2 | 1 |
| 28.6 | 1 |
| 28.9 | 2 |
| 29.3 | 11 |
| 29.5 | 1 |
| 30.1 | 4 |
| 30.4 | 4 |
| 30.8 | 1 |
| 31.1 | 1 |
| 31.6 | 2 |
| 31.8 | 2 |
| 32.3 | 3 |
| 33.1 | 6 |
| 33.4 | 1 |
| 33.7 | 1 |
| 34.4 | 2 |
| 34.6 | 13 |
| 34.9 | 7 |
| 35.2 | 1 |
| 35.6 | 9 |
| 35.8 | 4 |
| 36.5 | 2 |
| 37.2 | 1 |
| 37.5 | 2 |
| 39.0 | 1 |
| 39.5 | 1 |
| 39.9 | 1 |

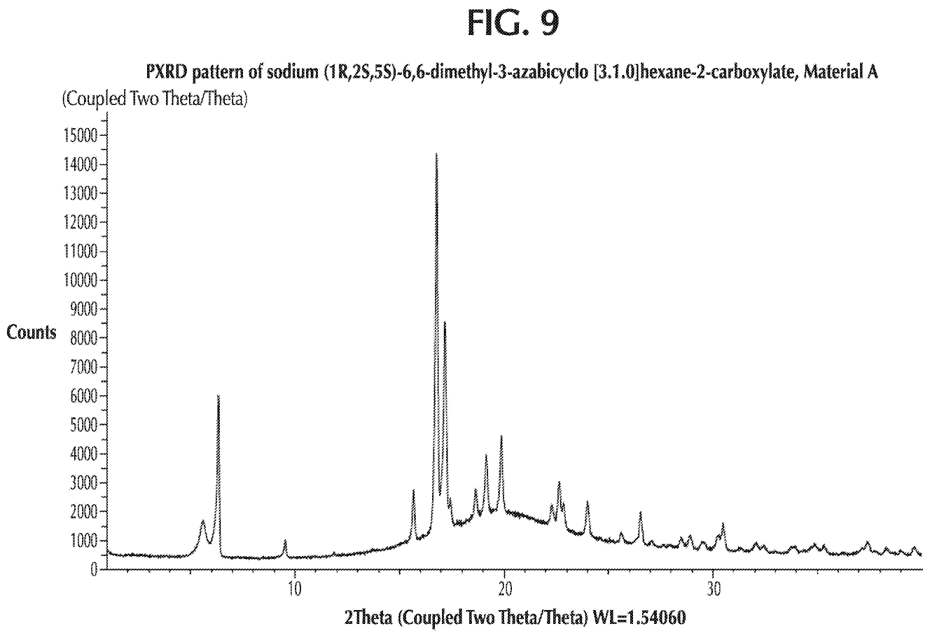

FIG. 9 provides the PXRD pattern for a form of sodium (1R,2S,5S)-6,6-dimethyl-3-azabicyclo[3.1.0]hexane-2-carboxylate designated Material A which is characterized by the PXRD peaks in the following table.

TABLE
PXRD peaks for sodium (1R,2S,5S)-6,6-dimethyl-3-azabicyclo
[3.1.0]hexane-2-carboxylate, Material A:

| Angle | Rel. |
|---|---|
| (degrees 2 THETA) | Intensity |
| 5.6 | 9 |
| 6.3 | 42 |
| 9.5 | 5 |
| 11.9 | 1 |
| 15.7 | 14 |
| 16.8 | 100 |
| 17.2 | 56 |
| 17.4 | 10 |
| 18.6 | 9 |
| 19.2 | 21 |
| 19.9 | 26 |
| 22.3 | 8 |
| 22.6 | 14 |
| 22.9 | 9 |
| 24.0 | 10 |
| 25.6 | 2 |
| 26.5 | 9 |
| 27.1 | 1 |
| 27.6 | 1 |
| 28.5 | 2 |
| 28.9 | 2 |
| 29.5 | 1 |
| 30.2 | 3 |
| 30.5 | 7 |
| 31.3 | 1 |
| 32.1 | 2 |
| 32.5 | 1 |
| 33.7 | 1 |
| 33.9 | 1 |
| 34.9 | 2 |
| 35.3 | 2 |
| 37.1 | 1 |
| 37.4 | 3 |
| 37.7 | 1 |
| 38.3 | 1 |
| 39.0 | 0 |
| 39.7 | 1 |

### Step 2: Preparation of (1R,2S,5S)-3-((S)-3,3-dimethyl-2-(2,2,2-trifluoroacetamido) butanoyl)-6,6-dimethyl-3-azabicyclo[3.1.0]hexane-2-carboxylic Acid, Compound IV

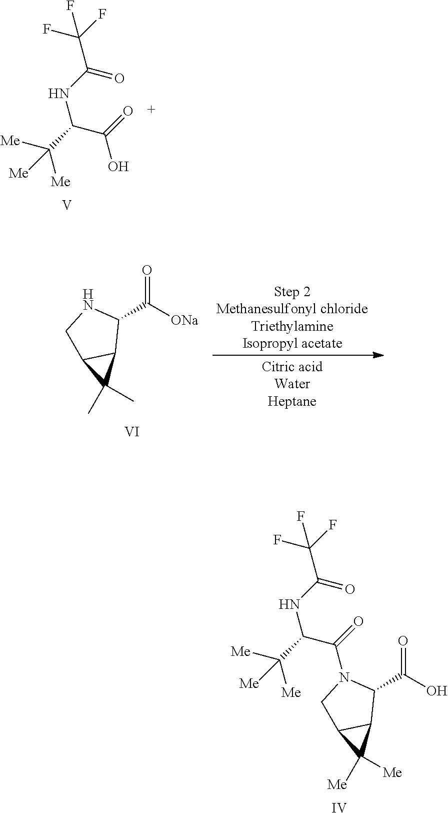

(S)-3,3-dimethyl-2-(2,2,2-trifluoroacetamido)butanoic acid, compound V (38.9 g, 169 mmol, 1.2 equivalents), methanesulfonyl chloride (17.8 g, 155 mmol, 1.1 equivalents) and isopropyl acetate (500 mL, 20 mL/g of compound V) are combined and stirred at 20° C. Triethylamine (49.0 mL, 423 mmol, 2.5 equivalents) is charged at a rate such that the reaction temperature does not exceed 25° C., and the resulting mixture stirred for 1 hour. Sodium (1R,2S,5S)-6,6-dimethyl-3-azabicyclo[3.1.0]hexane-2-carboxylate, compound VI (25.0 g, 141 mmol, 1.0 equivalents) is charged, and the mixture stirred for 4 hours. A sample of the reaction mixture is obtained and analyzed for reaction completion (not more than 3% VI by UPLC). If reaction is not complete, additional triethylamine may be added. The reaction mixture is quenched by addition of aqueous citric acid (74 g citric acid monohydrate, 353 mmol, 2.5 equivalents, in 150 ml water), and the mixture heated to 40° C. The mixture is stirred for at least 10 minutes, then the layers are allowed to settle. The aqueous phase is removed, and the organic phase is washed with 2 portions of water (125 mL each). The organic phase is cooled to 10-15° C. and concentrated by vacuum distillation (−100 mbar, gradually warming to a maximum jacket temperature of 60° C.) to a volume of approximately 192 mL. The mixture is analyzed for water content (Karl-Fischer); if greater than 3 wt % water, the vacuum distillation is repeated with additional isopropyl acetate. The solution is heated to 60° C. and heptane (192 mL) is added. The mixture is stirred and cooled to 10° C. at a rate of ˜−12° C./hour. The slurry is stirred at 10° C. for 3 hours. Solids are collected by filtration and rinsed with 1:1 iPrOAc/heptane (100 mL). The solids are dried at 50° C. in a vacuum oven to provide (1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid, compound IV.

¹H NMR (600 MHz, DMSO-d₆, 298K): δ 12.72 (s, 1H), 9.42 (d, 1H), 4.44 (d, 1H), 4.15 (s, 1H), 3.85 (dd, 1H), 3.72 (d, 1H), 1.53 (dd, 1H), 1.41 (d, 1H), 1.01 (s, 3H), 1.00 (s, 9H), 0.82 (s, 3H).

¹³C NMR (150 MHz, DMSO-d₆, 298K): d 172.3, 167.6, 156.9 (²JCF=37 Hz), 115.8 (²JCF=288 Hz), 59.2, 58.1, 47.2, 34.7, 29.7, 26.7, 26.2, 25.7, 18.8, 12.1.

Note: in the ¹H and ¹³C NMR spectra, 2 sets of resonances were observed due to the presence of both E and Z amide bond rotamers in solution. Only the major resonances (Z rotamer, 92%) are listed here.

HRMS: (ESI⁺) Calcd for C₁₆H₂₄F₃N₂O₄⁺: 365.1610, Found: 365.1684 (mass deviation +0.5 ppm)

### Preparation of Crystalline (1R,2S,5S)-3-((S)-3,3-dimethyl-2-(2,2,2-trifluoroacetamido) butanoyl)-6,6-dimethyl-3-azabicyclo[3.1.0]hexane-2-carboxylic Acid, Form 1, Prepared by a Seeded Crystallization Process From Aqueous Isopropanol

### Step 1

In a 100 mL reactor add (1R,2S,5S)-3-((S)-3,3-dimethyl-2-(2,2,2-trifluoroacetamido) butanoyl)-6,6-dimethyl-3-azabicyclo[3.1.0]hexane-2-carboxylic acid (5.08 g, 13.9 mmol) and Isopropanol (21.25 mL, 277.9 mmol) and stir at 500 rpm for 30 minutes to dissolve.

### Step 2: Reverse Addition

In a separate 100 mL reactor add water (75 mL, 4163.2 mmol) and isopropanol (3.75 mL, 49.0 mmol) (95-05 v/v %) then add (1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid (150 mg, 0.4116 mmol), as a seed and heat the mixture to 50° C. at a rate of 5° C./min and stir for 30 minutes. Add the (1R,2S,5S)-3-((S)-3,3-dimethyl-2-(2,2,2-trifluoro acetamido)butanoyl)-6,6-dimethyl-3-azabicyclo[3.1.0]hexane-2-carboxylic acid solution in isopropanol (from Step 1 above) over a period of 6 hours. Stir the resulting mixture 5 for 1 hour at 50° C. then cool the mixture to 10° C. at a rate of-0.1° C./min and stir overnight. The resulting slurry was then filtered and the solid was washed with a mixture of isopropanol (5 mL, 65.39 mmol) and water (15 mL, 832.64 mmol). The resulting solid was dried under vacuum at 60° C. overnight to provide crystalline (1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid, Form 1. The solid was characterized by 10 powder X-Ray diffraction (PXRD) according to the method described herein.

TABLE
Crystal data and structure refinement for (1R,2S,5S)-3-((S)-
3,3-dimethyl-2-(2,2,2-trifluoroacetamido) butanoyl)-6,6-dimethyl-
3-azabicyclo[3.1.0]hexane-2-carboxylic acid, Form 1:

| Identification code | E152 | |
|---|---|---|
| Crystallization | Ethanol/Water/Ethyl Acetate | |
| Empirical formula | C16 H23 F3 N2 O4 | |
| Formula weight | 364.36 | |
| Temperature | 296(2) K | |
| Wavelength | 1.54178 Å | |
| Crystal system | Trigonal | |
| Space group | R3 | |
| Unit cell dimensions | a = 14.1740(6) Å | α = 114.11°. |
| | b = 14.1740(6) Å | β = 114.11°. |
| | c = 14.1740(6) Å | γ = 114.11°. |

| | |
|---|---|
| Volume | 1715.9(4) Å³ |
| Z | 3 |
| Density (calculated) | 1.058 Mg/m³ |
| Absorption coefficient | 0.788 mm⁻¹ |
| F(000) | 576 |
| Crystal size | 0.220 × 0.100 × 0.100 mm³ |
| Theta range for data collection | 6.445 to 80.034°. |
| Index ranges | −17 <= h <= 16, −14 <= k <= |
| | 16, −14 <= l <= 17 |
| Reflections collected | 13310 |
| Independent reflections | 4011 [R(int) = 0.0369] |
| Completeness to theta = 67.679° | 98.9% |
| Absorption correction | Empirical |
| Refinement method | Full-matrix least-squares on F² |
| Data/restraints/parameters | 4011/6/244 |
| Goodness-of-fit on F² | 1.056 |
| Final R indices [I > 2sigma(I)] | R1 = 0.0582, wR2 = 0.1675 |
| R indices (all data) | R1 = 0.0611, wR2 = 0.1710 |
| Extinction coefficient | n/a |
| Largest diff. peak and hole | 0.292 and −0.174 e · Å⁻³ |

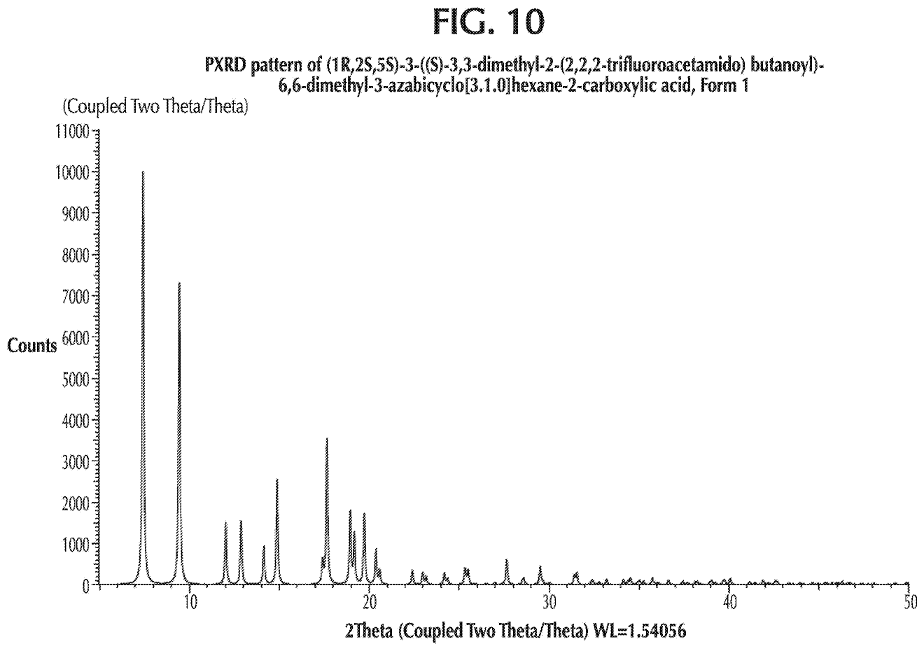

FIG. 10 provides the PXRD pattern for (1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid, Form 1 which is characterized by the PXRD peaks in the following table.

TABLE
PXRD peaks for (1R,2S,5S)-3-((S)-3,3-dimethyl-2-(2,2,2-
trifluoroacetamido) butanoyl)-6,6-dimethyl-3-azabicyclo[3.1.0]hexane-
2-carboxylic acid, Form 1:

| Angle | Rel. |
|---|---|
| (degrees 2 THETA) | Intensity |
| 7.4 | 100 |
| 9.4 | 73 |
| 12.0 | 15 |
| 12.9 | 16 |
| 14.1 | 9 |
| 14.9 | 26 |
| 17.4 | 7 |
| 17.7 | 35 |
| 19.0 | 18 |
| 19.2 | 12 |
| 19.7 | 17 |
| 20.4 | 9 |
| 20.6 | 4 |
| 22.4 | 3 |
| 23.0 | 3 |
| 24.2 | 3 |
| 24.4 | 2 |
| 25.3 | 4 |
| 25.5 | 4 |
| 27.6 | 6 |
| 28.6 | 1 |
| 29.5 | 4 |
| 31.4 | 2 |
| 31.5 | 3 |
| 32.4 | 1 |
| 33.2 | 1 |
| 34.1 | 1 |
| 34.5 | 2 |
| 35.7 | 1 |
| 36.1 | 0 |
| 37.4 | 1 |
| 38.3 | 1 |
| 39.0 | 1 |
| 39.7 | 1 |
| 40.1 | 1 |
| 41.2 | 1 |
| 41.9 | 1 |
| 42.6 | 1 |
| 46.3 | 1 |

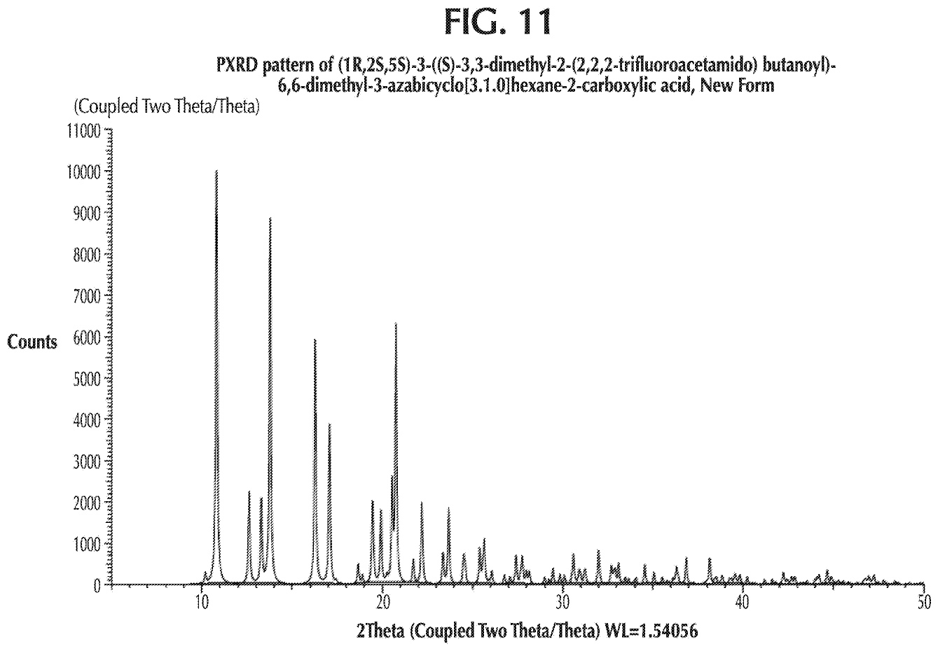

FIG. 11 depicts the PXRD pattern for (1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid, New form which is characterized by the PXRD peaks in the following table.

TABLE
PXRD peaks (degrees 2-theta) for (1R,2S,5S)-
3-((S)-3,3-dimethyl-2-(2,2,2-trifluoroacetamido)
butanoyl)-6,6-dimethyl-3-azabicyclo[3.1.0]hexane-
2-carboxylic acid, New form:

| Angle (2-Theta) | Rel. Intensity |
|---|---|
| 10.2 | 3 |
| 10.8 | 100 |
| 12.6 | 22 |
| 13.3 | 21 |
| 13.8 | 88 |
| 16.3 | 59 |
| 17.1 | 39 |
| 18.7 | 5 |
| 18.9 | 2 |
| 19.5 | 20 |
| 19.9 | 18 |
| 20.3 | 2 |
| 20.6 | 26 |
| 20.8 | 63 |
| 21.7 | 6 |
| 22.2 | 20 |
| 23.4 | 8 |
| 23.7 | 18 |
| 24.5 | 7 |
| 25.4 | 9 |
| 25.7 | 11 |
| 26.1 | 3 |
| 26.8 | 2 |
| 27.1 | 2 |
| 27.4 | 7 |
| 27.8 | 7 |
| 28.0 | 3 |
| 28.2 | 3 |
| 29.0 | 2 |
| 29.5 | 4 |
| 29.8 | 2 |
| 30.1 | 2 |
| 30.6 | 7 |
| 30.9 | 3 |
| 31.2 | 3 |
| 32.0 | 8 |
| 32.7 | 4 |
| 32.9 | 4 |
| 33.1 | 5 |
| 33.5 | 1 |
| 33.7 | 1 |
| 34.1 | 1 |
| 34.6 | 4 |
| 35.1 | 3 |
| 35.5 | 1 |
| 36.3 | 4 |
| 36.9 | 6 |
| 38.2 | 6 |
| 38.8 | 2 |
| 39.3 | 1 |
| 39.6 | 2 |
| 39.8 | 2 |
| 40.2 | 2 |
| 42.2 | 3 |
| 44.2 | 2 |
| 44.7 | 3 |
| 47.0 | 2 |
| 47.3 | 2 |

### Step 3: Comparator Process for of Preparation of (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido) butanoyl)-6,6-dimethyl-3-azabicyclo[3.1.0]hexane-2-carboxamide, Compound II

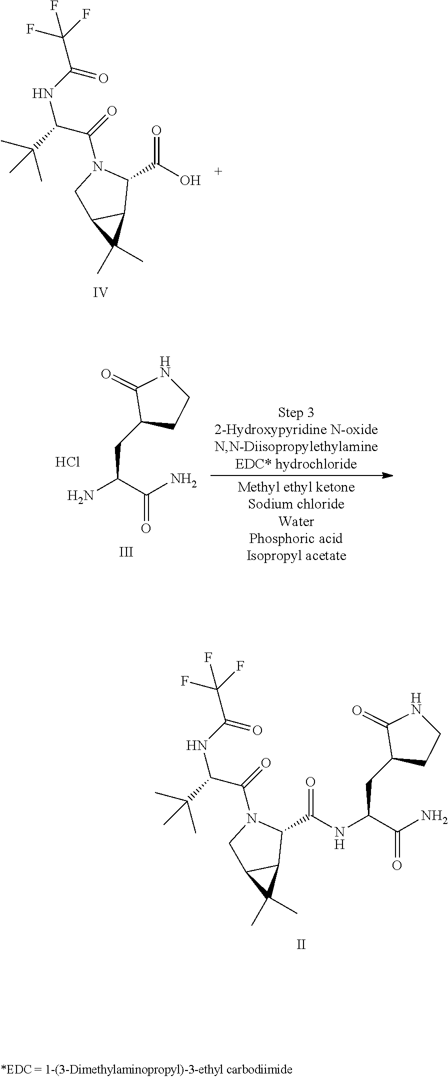

(1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid, compound IV (1.0 kg, 2.74 mol, 1.0 equivalents) and methyl ethyl ketone (5.0 L, 5 L/kg of compound IV) are combined and stirred at 25° C. 2-Hydroxypyridine N-oxide (0.228 kg, 2.05 mol, 0.75 equivalents) and triethylamine (0.416 kg, 4.11 mol, 1.50 equivalents) are added, and the resulting slurry is stirred for 30 minutes. (S)-2-amino-3-((S)-2-oxopyrrolidin-3-yl)propanamide hydrochloride, compound III (0.597 kg, 2.87 mol, 1.05 equivalents) and 1-(3-dimethylaminopropyl)-3-ethyl-carbodiimide hydrochloride (EDC, 0.682 kg, 3.56 mol, 1.30 equivalents) are charged, and stirring is maintained for 16 hours. The reaction is sampled for completion (target of not more than 1.0% of compound IV remaining). If the reaction is not complete, the mixture is stirred for additional time. The reaction is quenched by the addition of aqueous NaCl (3.0 L of a 14 wt % brine solution) and stirring is maintained for 30 minutes. Stirring is stopped and the layers allowed to settle. The lower aqueous phase is removed, and the organic phase is washed with a second portion of aqueous NaCl (3.0 L of a 14 wt % brine solution), following the same protocol. The organic phase is then concentrated by vacuum distillation at 0.3 bar while adding additional isopropyl acetate (13 L) to maintain constant volume of ˜6 L/kg, ending the distillation at 7 L/kg. A sample is analyzed for water content (Karl-Fischer) with a target of not more than 0.2 wt % water. The resulting organic solution of (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide, compound II is used in Step 4 without further purification.

### Preparation and Isolation of (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide, Methyl Ethyl Ketone (MEK) Solvate, II MEK Solvate

A sample of the title MEK solvate was isolated by the following procedure. The Step 3 reaction was performed as described above on 150 g scale and allowed to stir for 3 days. The resulting slurry was combined with aqueous NaCl (325 mL of a 14 wt % solution), warmed to 48° C. and stirred, resulting in two clear phases. The aqueous phase was removed, and the organic phase was washed with a second portion of aqueous NaCl (400 mL of a 14 wt % solution). The organic phase was concentrated at 45° C. under partial vacuum and additional MEK was added. This was repeated until the water content was reduced to 6% and the volume was ˜4 mL/g of starting material. The mixture was cooled gradually to 15° C. and held for 3 hours. The resulting solids were collected by filtration, rinsed with MEK, and dried in a vacuum oven, providing 132 g of (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide, methyl ethyl ketone solvate.

¹H NMR (600 MHz, DMSO-d₆, 298K): δ 9.40 (d, J=8.6 Hz, 1H), 8.76 (d, J=9.2 Hz, minor), 8.48 (d, J=7.2 Hz, minor), 8.28 (d, J=8.9 Hz, 1H), 7.65 (s, minor), 7.55 (s, 1H), 7.45-7.39 (m, minor), 7.35-7.26 (m, 1H), 7.06-6.99 (m, 1H), 6.92 (s, minor), 4.49 (s, minor), 4.43 (d, J=8.6 Hz, 1H), 4.33-4.28 (m, 1H), 4.28 (s, 1H), 4.21 (d, J=9.2 Hz, minor), 4.09 (ddd, J=11.1, 7.2, 3.8 Hz, minor), 3.89 (dd, J=10.3, 5.5 Hz, 1H), 3.67 (d, J=10.4 Hz, 1H), 3.58 (dd, J=12.4, 5.6 Hz, minor), 3.54 (dd, minor), 3.40 (d, J =12.5 Hz, minor), 3.16-3.10 (m, 1H), 3.03 (td, J=9.4, 7.1 Hz, 1H), 2.46-2.36 (m, 3H), 2.18-2.10 (m, 1H), 2.07 (s, 3H), 2.02 (m, minor), 1.94 (ddd, J=13.5, 12.0, 3.6 Hz, 1H), 1.73 (dt, J=12.4, 9.1 Hz, minor), 1.69-1.59 (m, 1H), 1.54-1.46 (m, 2H), 1.38 (d, J=7.6 Hz, 1H), 1.19 (t, J=7.3 Hz, minor), 1.02 (s, 3H), 0.98 (s, 9H), 0.91 (t, J=7.3 Hz, 3H), 0.84 (s, 3H).

¹³C NMR (150 MHz, DMSO-d₆, 298K): δ 208.83, 178.92 (minor), 178.62, 173.74 (minor), 173.51, 170.63, 170.52 (minor), 167.94 (minor), 167.21, 156.87 (q, J=36.9 Hz), 155.52 (d, J=36.7 Hz-minor), 118.96-112.77 (m), 60.61 (minor), 60.25, 58.11, 57.67 (minor), 51.78 (minor), 50.32, 47.70, 47.50 (minor), 45.51 (minor), 42.09 (minor), 40.06, 39.29, 37.88 (minor), 37.32, 36.48 (minor), 36.16, 35.85, 34.67, 34.55, 34.09, 33.58 (minor), 30.57, 29.35, 27.65 (minor), 27.39, 27.10, 26.40, 26.28, 25.87, 24.86 (minor), 19.05 (minor), 18.58, 13.24 (minor), 12.38, 8.51 (minor), 7.66.

Note: in the ¹H and ¹³C NMR spectra, 2 sets of resonances were observed due to the presence of both E and Z amide bond rotamers in solution. Where relevant, the less abundant rotamer signals are designated as “minor.”

HRMS: (ESI⁺) Calcd for C₂₃H₃₅F₃N₅O₅⁺: 518.2585, Found: 518.2582 (mass deviation −0.46 ppm)

### Alternative Step 3: Optimized Process for Preparation of (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide

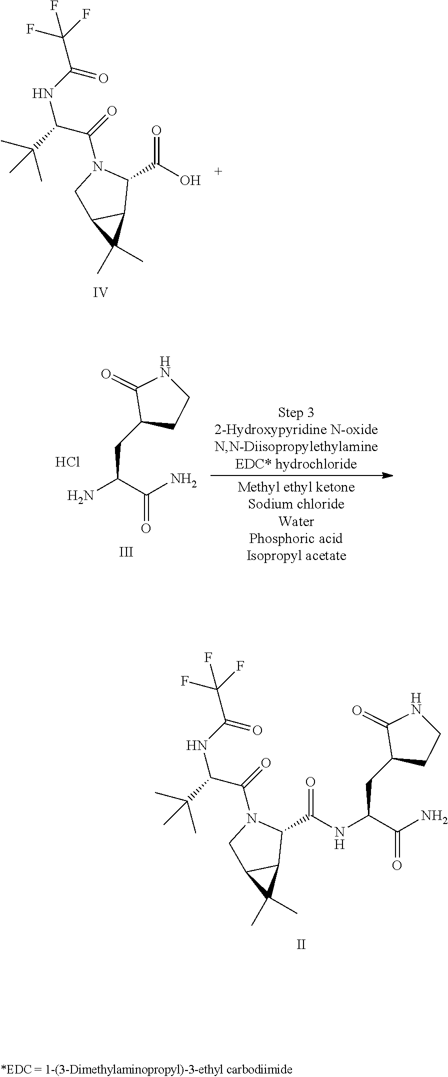

In reactor A, (1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid, compound IV (1.0 kg, 2.74 mol, 1.0 equivalents) and methyl ethyl ketone (2.0 L, 2 L/kg of compound IV) are combined and stirred at 25° C. 2-Hydroxypyridine N-oxide (0.274 kg, 2.47 mol, 0.90 equivalents) and triethylamine (0.694 kg, 6.86 mol, 2.50 equivalents) are added, the resulting slurry is stirred for 30 minutes then warmed up to 50° C. In reactor B, (S)-2-amino-3-((S)-2-oxopyrrolidin-3-yl)propanamide hydrochloride, compound III (0.597 kg, 2.87 mol, 1.05 equivalents) and 1-(3-dimethylaminopropyl)-3-ethyl-carbodiimide hydrochloride (EDC, 0.682 kg, 3.56 mol, 1.30 equivalents) are combined with methyl ethyl ketone (3.0 L, 3 L/kg compound IV), and the resulting slurry is stirred for 30 min then warmed up to 50° C. The now solution from reactor A is then transferred into reactor B while maintaining the temperature at 50° C. in reactor B and stirring is continued for at least 6 hours. The reaction is sampled for completion (target of not more than 3% compound IV remains unreacted). If the reaction is not complete, the mixture is stirred for additional time. The reaction is quenched at 50° C. by the addition of aqueous NaCl (3.0 L of a 14 wt % brine solution, 3.0 L/kg of compound IV) and stirring is maintained for 30 minutes. Stirring is stopped and the layers allowed to settle. The lower aqueous phase is removed, and the organic phase is washed with a second portion of aqueous NaCl (3.0 L of a 14 wt % brine solution), following the same protocol. The organic phase is cooled down then concentrated by vacuum distillation at 0.3 bar while adding additional isopropyl acetate (18 L, 18 L/kg compound IV) to maintain constant volume of ˜6 L/kg, ending the distillation at 8 L/kg. A sample is analyzed for water content (Karl-Fischer) with a target of not more than 0.2 wt % water. The resulting organic solution of (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide is used in Step 4 without further purification.

The (S)-2-amino-3-((S)-2-oxopyrrolidin-3-yl)propanamide hydrochloride, compound III employed in the previous reactions can exist in certain solid forms.

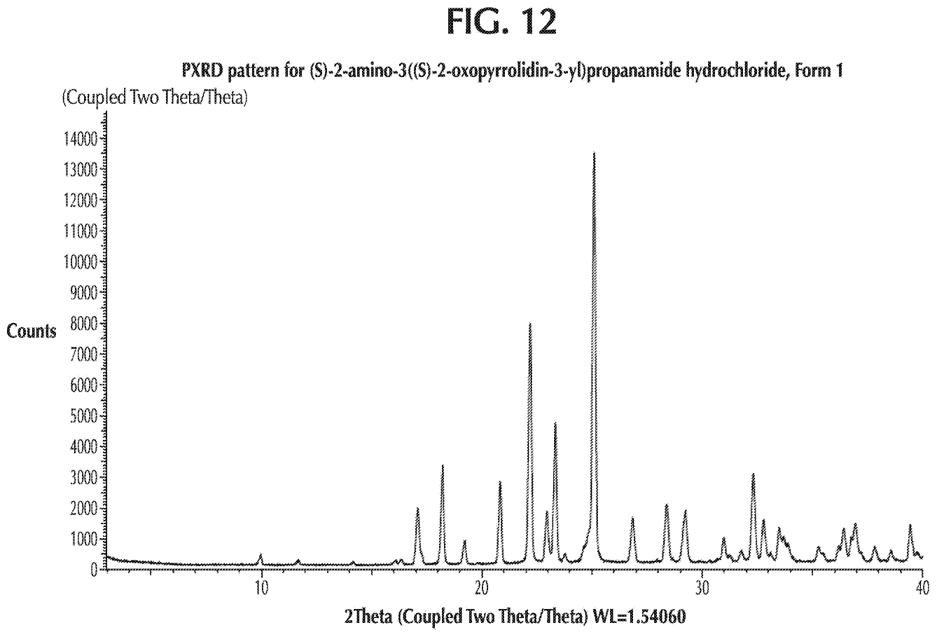

FIG. 12 provides the PXRD pattern for(S)-2-amino-3-((S)-2-oxopyrrolidin-3-yl)propanamide hydrochloride, Form 1 which is characterized by the PXRD peaks in the following table.

TABLE
PXRD peaks for (S)-2-amino-3-((S)-2-oxopyrrolidin-
3-yl)propanamide hydrochloride, Form 1:

| Angle | Rel. |
|---|---|
| (degrees 2 THETA) | Intensity |
| 9.97 | 3 |
| 11.67 | 1 |
| 14.17 | 1 |
| 16.08 | 1 |
| 16.35 | 1 |
| 17.10 | 14 |
| 17.27 | 3 |
| 18.23 | 24 |
| 19.21 | 4 |
| 20.83 | 20 |
| 22.20 | 58 |
| 22.97 | 12 |
| 23.35 | 34 |
| 23.79 | 2 |
| 24.62 | 3 |
| 25.10 | 100 |
| 26.85 | 11 |
| 28.39 | 14 |
| 29.24 | 13 |
| 30.98 | 6 |
| 31.78 | 2 |
| 32.32 | 23 |
| 32.79 | 10 |
| 33.10 | 1 |
| 33.50 | 6 |
| 33.70 | 4 |
| 33.90 | 3 |
| 35.27 | 3 |
| 36.20 | 3 |
| 36.42 | 6 |
| 36.75 | 6 |
| 36.95 | 7 |
| 37.83 | 3 |
| 38.58 | 2 |
| 39.44 | 7 |
| 39.75 | 1 |

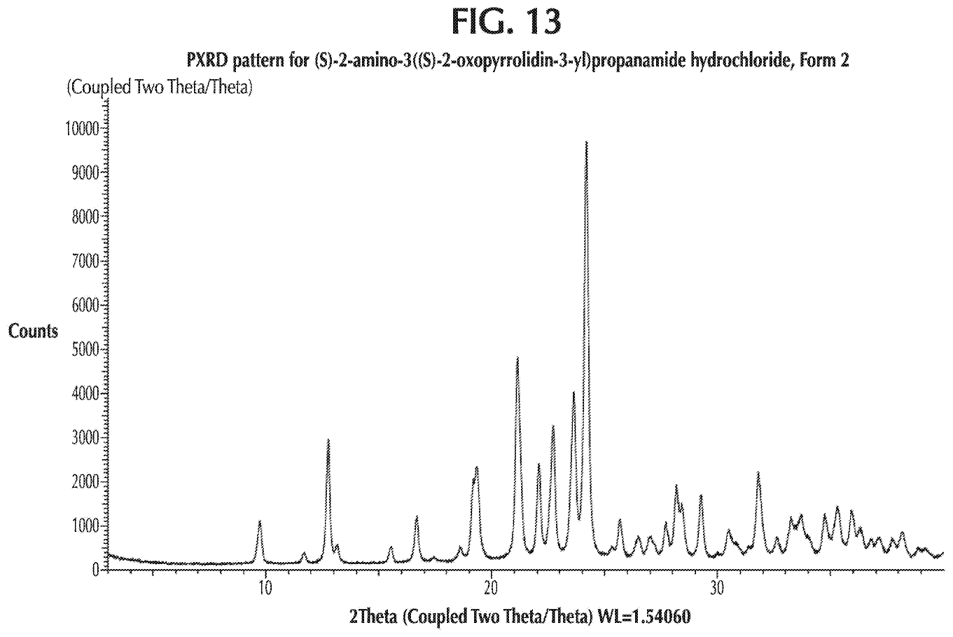

FIG. 13 provides the PXRD pattem for(S)-2-amino-3-((S)-2-oxopyrrolidin-3-yl)propanamide hydrochloride, Form 2 which is characterized by the PXRD peaks in the following table.

TABLE
PXRD peaks for (S)-2-amino-3-((S)-2-oxopyrrolidin-
3-yl)propanamide hydrochloride, Form 2:

| Angle | Rel. |
|---|---|
| (degrees 2 THETA) | Intensity |
| 9.74 | 9 |
| 11.70 | 2 |
| 12.76 | 30 |
| 13.15 | 4 |
| 15.55 | 4 |
| 16.67 | 11 |
| 17.44 | 1 |
| 18.61 | 3 |
| 19.19 | 20 |
| 19.33 | 23 |
| 21.15 | 49 |
| 22.08 | 22 |
| 22.72 | 32 |
| 23.63 | 39 |
| 24.19 | 100 |
| 25.31 | 2 |
| 25.67 | 9 |
| 26.48 | 5 |
| 27.03 | 4 |
| 27.71 | 8 |
| 28.17 | 17 |
| 28.40 | 13 |
| 29.26 | 15 |
| 30.49 | 6 |
| 30.84 | 3 |
| 31.38 | 2 |
| 31.81 | 20 |
| 32.61 | 3 |
| 33.24 | 9 |
| 33.70 | 9 |
| 34.01 | 4 |
| 34.73 | 9 |
| 35.30 | 11 |
| 35.93 | 9 |
| 36.30 | 6 |
| 36.79 | 3 |
| 37.12 | 3 |
| 37.76 | 3 |
| 38.17 | 6 |
| 38.87 | 2 |
| 39.20 | 2 |

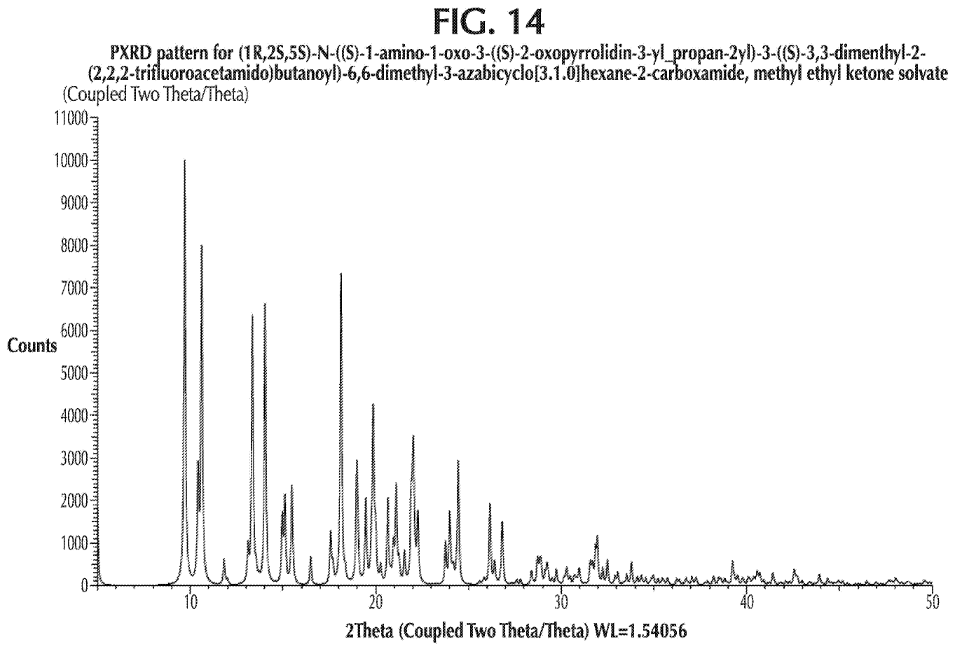

FIG. 14 provides the PXRD pattern for (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide, methyl ethyl ketone (MEK) solvate which is characterized by the PXRD peaks in the following table.

TABLE
PXRD peaks for (1R,2S,5S)-N-((S)-1-amino-1-oxo-
3-((S)-2-oxopyrrolidin-3-yl)propan-2-yl)-3-((S)-
3,3-dimethyl-2-(2,2,2-trifluoroacetamido)butanoyl)-
6,6-dimethyl-3-azabicyclo[3.1.0]hexane-2-carboxamide,
methyl ethyl ketone solvate:

| Angle | Rel. |
|---|---|
| (degrees 2 THETA) | Intensity |
| 9.7 | 100 |
| 10.4 | 29 |
| 10.6 | 80 |
| 11.8 | 6 |
| 12.0 | 1 |
| 13.1 | 10 |
| 13.4 | 63 |
| 14.0 | 67 |
| 15.0 | 17 |
| 15.1 | 21 |
| 15.5 | 23 |
| 16.5 | 7 |
| 17.6 | 12 |
| 17.7 | 6 |
| 18.1 | 73 |
| 18.4 | 5 |
| 19.0 | 29 |
| 19.5 | 20 |
| 24.2 | 5 |
| 24.5 | 29 |
| 25.6 | 1 |
| 25.9 | 2 |
| 26.2 | 19 |
| 26.4 | 5 |
| 26.8 | 15 |
| 27.6 | 1 |
| 27.8 | 1 |
| 28.4 | 3 |
| 28.7 | 6 |
| 28.9 | 6 |
| 29.2 | 5 |
| 29.7 | 3 |
| 30.2 | 2 |
| 30.3 | 4 |
| 30.5 | 2 |
| 30.7 | 2 |
| 35.0 | 2 |
| 35.2 | 1 |
| 35.5 | 1 |
| 35.7 | 1 |
| 36.2 | 1 |
| 36.4 | 1 |
| 36.7 | 2 |
| 37.1 | 2 |
| 37.3 | 2 |
| 38.2 | 2 |
| 38.5 | 1 |
| 38.8 | 1 |
| 39.3 | 5 |
| 39.5 | 2 |
| 39.8 | 1 |
| 40.1 | 1 |
| 40.6 | 3 |
| 41.4 | 3 |

TABLE
Crystal data and structure refinement for (1R,2S,5S)-
N-((S)-1-amino-1-oxo-3-((S)-2-oxopyrrolidin-3-yl)propan-
2-yl)-3-((S)-3,3-dimethyl-2-(2,2,2-trifluoroacetamido)
butanoyl)-6,6-dimethyl-3-azabicyclo[3.1.0]hexane-
2-carboxamide, methyl ethyl ketone solvate:

| Identification code | E222 | |
|---|---|---|
| Empirical formula | C27 H42 F3 N5 O6 | |
| Formula weight | 589.65 | |
| Temperature | 273.15 K | |
| Wavelength | 1.54178 Å | |
| Crystal system | Orthorhombic | |
| Space group | P2₁2₁2₁ | |
| Unit cell dimensions | a = 9.4471(3) Å | α = 90°. |
| | b = 9.6463(3) Å | β = 90°. |
| | c = 35.4946(12) Å | γ = 90°. |

| | |
|---|---|
| Volume | 3234.61(18) Å³ |
| Z | 4 |
| Density (calculated) | 1.211 Mg/m³ |
| Absorption coefficient | 0.821 mm⁻¹ |
| F(000) | 1256 |
| Crystal size | 0.202 × 0.174 × 0.124 mm³ |
| Theta range for data collection | 4.750 to 79.276°. |
| Index ranges | −11 <= h <= 8, −12 <= k <= |
| | 11, −45 <= l <= 43 |
| Reflections collected | 20328 |
| Independent reflections | 6587 [R(int) = 0.0534] |
| Completeness to theta = 67.679° | 99.6% |
| Absorption correction | None |
| Max. and min. transmission | 0.7543 and 0.5564 |
| Refinement method | Full-matrix least-squares on F² |
| Data/restraints/parameters | 6587/5/397 |
| Goodness-of-fit on F² | 1.016 |
| Final R indices [I > 2sigma(I)] | R1 = 0.0718, wR2 = 0.2067 |
| R indices (all data) | R1 = 0.0844, wR2 = 0.2218 |
| Absolute structure parameter | 0.14(16) |
| Extinction coefficient | n/a |
| Largest diff. peak and hole | 0.487 and −0.220 e · Å⁻³ |

### Step 4: Preparation of (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Methyl t-butyl Ether Solvate, I′

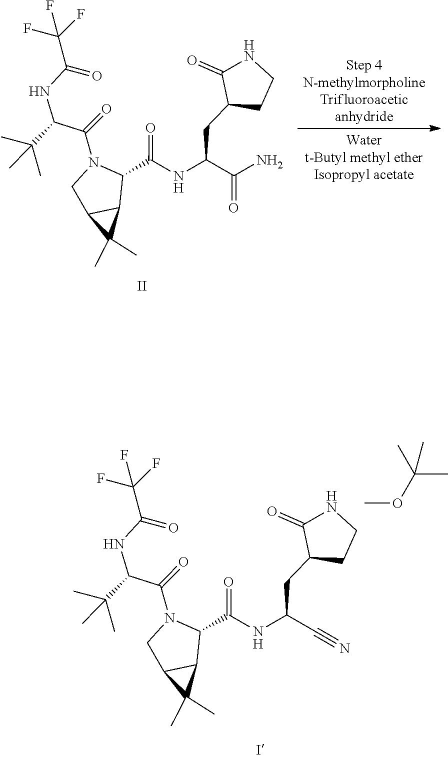

The isopropyl acetate solution of (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide, compound II prepared above in Step 4 (assumed quantitate conversion, 2.74 mol, 1.0 equivalents) is combined with N-methylmorpholine (1.11 kg, 10.4 mol, 4.0 equivalents) and stirred at 20° C. Trifluoroacetic anhydride (1.15 kg, 5.20 mol, 2.0 equivalents) is charged over 60 minutes, maintaining the reaction temperature at not more than 25° C. The resulting mixture is stirred for 1 hour. A sample is analyzed for reaction completion (not more than 0.5% of compound II remaining). If the reaction is not complete, maintain stirring for another 60 minutes, and charge additional trifluoroacetic anhydride if needed. The reaction is quenched by addition of water (3.0 L, 3.0 L/kg of compound IV from previous step), stirring is maintained for 30 min, then stopped and the layers allowed to settle. The aqueous phase is removed, and the organic phase washed with a second 3.0 L portion of water. The organic phase is then concentrated by vacuum distillation (0.1 bar) to a volume of 3.5 L (3.5 L/kg of compound IV from previous step). Isopropyl acetate (5.0 L, 5.0 L/kg of compound IV from previous step) is added, and the solution is concentrated by vacuum distillation to a volume of 3.5 L (3.5 L/kg of compound IV from previous step). This solution is stirred at 50° C., and methyl t-butyl ether (MTBE) is added over 60 minutes. If product crystallization does not occur during this addition, PF-07321332 MTBE solvate seed (10 g, 1.0 wt % based on compound IV from previous step) may be added. An additional portion of MTBE (6.0 L, 6.0 L/kg of compound IV from previous step) is added over 3 hours. This slurry is stirred at 50° C. for 1 hour, cooled to 20° C. at a rate of 0.1 K/min, and stirred at 20° C. for 2 hours. Solids are collected by filtration, rinsed with 2.0 L/kg of compound IV from previous step 80:20 MTBE: iPrOAc solution, and dried at not more than 50° C.

A form of (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, isopropyl acetate solvate was obtained according to the following procedure.

To a 50 mL reactor equipped with magnetic drive stirrer was charged PF-07328615 (in methyl ethyl ketone) (7.87 g, 15.2 mmol). Some solids have crystallized from the PF-07328615 solution. To this was added isopropyl acetate (for distillation) (72 mL, 614.04 mmol) while performing a constant volume distillation with Tj-Tr set to 15° C., Tj max set to 70° C., and vacuum set to 300 mbar (actual vacuum is 295-305 mbar). Dilute to 7.5 mL/g (42 mL) with isopropyl acetate. Cool to 20° C. (Tj), set stir rate to 350 rpm then add 1-Methylmorpholine (6.7 mL, 61 mmol) over 5 min followed by addition of trifluoroacetic anhydride (4.28 mL, 30.4 mmol) via syringe pump over 1 hour and stir for one hour after the addition finishes. The reaction mixture was quenched with water (17 mL, 943.66 mmol), transferred to a separatory funnel and the aqueous layer was removed. The organic layer was returned to the reactor and distilled to a concentration of 3.5 mL/g (20 mL) with Tj-Tr set to 15° C., Tj max set to 70° C., and vacuum set to 100 mbar (actual vacuum is 100-105 mbar). To this was added isopropyl acetate (28 mL, 238.79 mmol) and the mixture was again distilled to a concentration of 3.5 mL/g. Solids crystallized when the volume reached ˜30 mL. Filter 1 mL of solution (yields 71 mg of wet solids) and analyze by PXRD (see FIG. 15).

FIG. 15 provides the PXRD pattern for (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, isopropyl acetate solvate which is characterized by the PXRD peaks in the following statement.

TABLE
PXRD peaks for (1R,2S,5S)-N-{(1S)-1-cyano-2-[(3S)-
2-oxopyrrolidin-3-yl]ethyl}-6,6-dimethyl-3-[3-
methyl-N-(trifluoroacetyl)-L-valyl]-3-azabicyclo[3.1.0]hexane-
2-carboxamide, isopropyl acetate solvate

| Angle (°2-theta) | Rel. Intensity (%) |
|---|---|
| 6.1 | 34 |
| 11.2 | 21 |
| 11.3 | 13 |
| 11.6 | 17 |
| 12.2 | 14 |
| 12.6 | 13 |
| 13.5 | 7 |
| 13.8 | 6 |
| 14.3 | 17 |
| 14.4 | 8 |
| 14.7 | 5 |
| 15.6 | 100 |
| 16.2 | 3 |
| 16.4 | 14 |
| 16.7 | 8 |
| 16.7 | 9 |
| 16.9 | 17 |
| 17.2 | 41 |
| 17.6 | 5 |
| 17.9 | 6 |
| 18.3 | 13 |
| 18.8 | 26 |
| 19.2 | 64 |
| 19.3 | 79 |
| 19.7 | 30 |
| 20.0 | 39 |
| 20.6 | 54 |
| 20.9 | 11 |
| 21.4 | 6 |
| 21.9 | 8 |
| 22.4 | 9 |
| 22.4 | 7 |
| 22.8 | 28 |
| 22.9 | 26 |
| 23.2 | 3 |
| 23.7 | 8 |
| 24.3 | 7 |
| 24.5 | 19 |
| 24.7 | 16 |
| 25.2 | 12 |
| 25.4 | 7 |
| 25.7 | 7 |
| 26.3 | 6 |
| 26.3 | 7 |
| 27.4 | 6 |
| 28.0 | 6 |
| 28.2 | 6 |
| 28.6 | 5 |
| 29.0 | 8 |
| 29.6 | 6 |
| 29.8 | 5 |
| 30.4 | 8 |
| 30.7 | 7 |
| 31.9 | 5 |
| 32.7 | 9 |
| 33.2 | 3 |
| 33.6 | 4 |
| 34.1 | 7 |
| 35.6 | 3 |
| 36.4 | 3 |
| 36.6 | 4 |
| 36.9 | 4 |
| 37.3 | 5 |
| 38.0 | 3 |
| 38.7 | 3 |

1. The compound (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo [3.1.0]hexane-2-carboxamide, methyl ethyl ketone solvate.

2. A process for preparing (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide, compound II 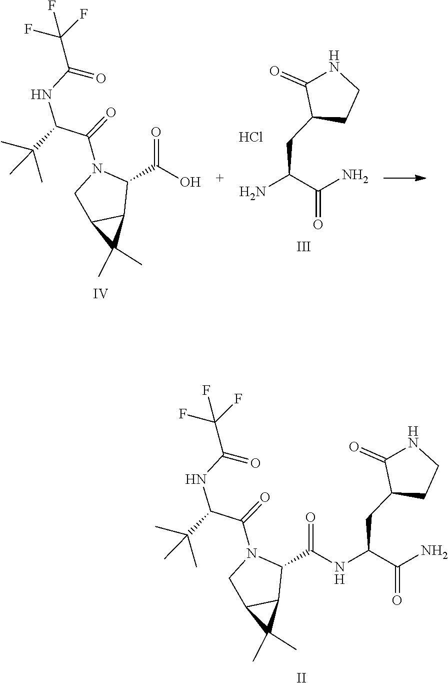
the process of reacting the compound of formula IV with the compound of formula III comprising the steps (a)-(d):
(a) (1R,2S,5S)-3-((S)-3,3-dimethyl-2-(2,2,2-trifluoroacetamido)butanoyl)-6,6-dimethyl-3-azabicyclo [3.1.0]hexane-2-carboxylic acid, compound IV and methyl ethyl ketone are combined followed by addition of 2-Hydroxypyridine N-oxide and triethylamine to provide a first mixture, S3-M1;
(b) (S)-2-amino-3-((S)-2-oxopyrrolidin-3-yl)propanamide hydrochloride, compound III, 1-(3-dimethyl aminopropyl)-3-ethyl-carbodiimide hydrochloride and methyl ethyl ketone are combined to provide a second mixture, S3-M2;
(c) combining the first mixture S3-M1 from step (a) with the second mixture S3-M2 from step (b) to provide a third mixture S3-M3; and
(d) stirring the third mixture S3-M3 from step (c) to provide the compound (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo [3.1.0]hexane-2-carboxamide, compound II.

3. The process of claim 2 wherein in step (a) the first mixture, S3-M1, comprises 1.0 equivalents of (1R,2S,5S)-3-((S)-3,3-dimethyl-2-(2,2,2-trifluoroacetamido) butanoyl)-6,6-dimethyl-3-azabicyclo [3.1.0]hexane-2-carboxylic acid, compound IV, 2 L of methyl ethyl ketone per kg of compound IV, 0.9 equivalents of 2-Hydroxypyridine N-oxide and 2.50 equivalents of triethylamine.

4. The process of claim 3 wherein in step (a) the first mixture, S3-M1, is prepared at about 25° C. and is stirred for about 30 minutes at about 25° C. and then is warmed to about 50° C.

5. The process of claim 4 wherein in step (b) the second mixture, S3-M2, comprises 1.05 equivalents of (S)-2-amino-3-((S)-2-oxopyrrolidin-3-yl)propanamide hydrochloride, compound III, 1.30 equivalents of 1-(3-dimethyl aminopropyl)-3-ethyl-carbodiimide hydrochloride and 3 L of methyl ethyl ketone per kg of compound IV.

6. The process claim 5 wherein in step (b) the second mixture, S3-M2, is prepared at about 25° C. and is stirred for 30 minutes at about 25° C. and then is warmed to about 50° C.

7. The process of claim 6 wherein in step (c) the first mixture, S3-M1, from step (a) is at about 50° C. and is combined with the second mixture, S3-M2, from step (b) which is at about 50° C. to provide the third mixture, S3-M3, while maintaining the temperature of the third mixture, S3-M3, at about 50° C.

8. The process of claim 7 wherein in step (d) the third mixture, S3-M2, from step (c) is stirred for at least 6 hours at about 50° C.

9. A process for preparing (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide, compound II 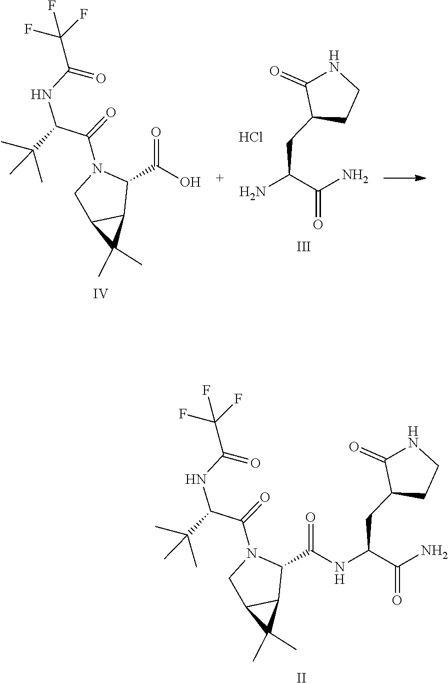
the process of reacting the compound of formula IV with the compound of formula III comprising the steps (a)-(d):
(a) (1R,2S,5S)-3-((S)-3,3-dimethyl-2-(2,2,2-trifluoroacetamido) butanoyl)-6,6-dimethyl-3-azabicyclo[3.1.0]hexane-2-carboxylic acid, compound IV (1.0 equivalent) and methyl ethyl ketone (2 L/kg of compound IV) at about 25° C. are combined followed by addition of 2-Hydroxypyridine N-oxide (0.90 equivalents) and triethylamine (2.50 equivalents), to provide a first mixture, S3-M1, which is stirred for about 30 minutes and then is warmed to about 50° C.;
(b) (S)-2-amino-3-((S)-2-oxopyrrolidin-3-yl)propanamide hydrochloride, compound III, (1.05 equivalents), 1-(3-dimethyl aminopropyl)-3-ethyl-carbodiimide hydrochloride (1.30 equivalents) and methyl ethyl ketone (3 L/kg of compound IV) are combined to provide a second mixture, S3-M2, which is stirred for about 30 minutes then warmed to about 50° C.;
(c) combining the first mixture, S3-M1, from step (a) with the second mixture, S3-M2, from step (b) while maintaining the temperature at about 50° C. to provide a third mixture, S3-M3; and
(d) stirring the third mixture, S3-M3, from step (c) at about 50° C. for at least 6 hours to provide the compound (1R,2S,5S)-N-((S)-1-amino-1-oxo-3-((S)-2-oxopyrrolidin-3-yl)propan-2-yl)-3-((S)-3,3-dimethyl-2-(2,2,2-trifluoroacetamido)butanoyl)-6,6-dimethyl-3-azabicyclo [3.1.0]hexane-2-carboxamide, compound II.

10. The process of claim 9 wherein the amount of acylurea impurities IMP-S3-3, (2S,4S)-4-(2-aminoethyl)-5-oxopyrrolidine-2-carboxamide, and IMP-S3-4, (1R,2S,5S)-N-(2-((3S,5S)-5-carbamoyl-2-oxopyrrolidin-3-yl)ethyl)-3-((S)-3,3-dimethyl-2-(2,2,2-trifluoroacetamido)butanoyl)-6,6-dimethyl-3-azabicyclo [3.1.0]hexane-2-carboxamide formed is not more than 10%.

11. The process of claim 9 wherein the amount of acylurea impurities IMP-S3-3, (2S,4S)-4-(2-aminoethyl)-5-oxopyrrolidine-2-carboxamide, and IMP-S3-4, (1R,2S,5S)-N-(2-((3S,5S)-5-carbamoyl-2-oxopyrrolidin-3-yl)ethyl)-3-((S)-3,3-dimethyl-2-(2,2,2-trifluoroacetamido)butanoyl)-6,6-dimethyl-3-azabicyclo [3.1.0]hexane-2-carboxamide formed is not more than 5%.

12. The process of claim 11 wherein the amount of rearrangement impurity IMP-S3-3, (2S,4S)-4-(2-aminoethyl)-5-oxopyrrolidine-2-carboxamide formed is not more than 2%.

13. The process of claim 11 wherein the amount of rearrangement impurity IMP-S3-4, (1R,2S,5S)-N-(2-((3S,5S)-5-carbamoyl-2-oxopyrrolidin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide formed is not more than 2%.

14. A process for preparing (1R,2S,5S)-3-((S)-3,3-dimethyl-2-(2,2,2-trifluoroacetamido)butanoyl)-6,6-dimethyl-3-azabicyclo [3.1.0]hexane-2-carboxylic acid, compound IV 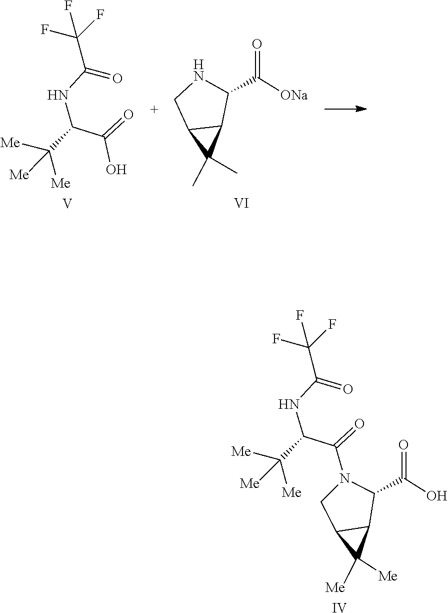
the process of reacting the compound of formula V with the compound of formula VI comprising the steps (a) to (d):
(a) combining(S)-3,3-dimethyl-2-(2,2,2-trifluoroacetamido)butanoic acid (compound V), methanesulfonyl chloride and isopropyl acetate to provide a first mixture, S2-M1;
(b) adding triethylamine to the first mixture, S2-M1, to provide a second mixture, S2-M2;
(c) adding Sodium (1R,2S,5S)-6,6-dimethyl-3-azabicyclo[3.1.0] hexane-2-carboxylate, compound VI, to the second mixture, S2-M2, to provide a third mixture, S2-M3; and
(d) stirring the third mixture, S2-M3, to provide (1R,2S,5S)-3-((S)-3,3-dimethyl-2-(2,2,2-trifluoroacetamido)butanoyl)-6,6-dimethyl-3-azabicyclo [3.1.0]hexane-2-carboxylic acid, compound IV.

15. The process of claim 14 wherein the first mixture, S2-M1, in step (a) comprises 1.2 equivalents of (S)-3,3-dimethyl-2-(2,2,2-trifluoroacetamido) butanoic acid, compound V, 1.1 equivalents of methanesulfonyl chloride and 20 mL of isopropyl acetate per g of compound V.

16. The process of claim 15 wherein in step (b) 2.5equivalents of triethylamine is added to the first mixture, S2-M1 which is at about 20° C., at a rate such that the temperature does not exceed 25° C. to provide the second mixture S2-M2.

17. The process of claim 16 wherein in step (c) 1.0 equivalents of Sodium (1R,2S,5S)-6,6-dimethyl-3-azabicyclo[3.1.0]hexane-2-carboxylate, compound VI, is added to the second mixture, S2-M2, to provide the third mixture S2-M3 which is stirred for about 4 hours.

18. The process of claim 17 wherein 2.5 equivalents of aqueous citric acid is added to the third mixture, S2-M3, and the resulting mixture is stirred for at least 10 minutes at about 40° C.

19. The process of claim 18 wherein the organic and aqueous layers of the resulting mixture are allowed to settle and the organic isopropyl acetate layer is separated from the aqueous layer, washed with water and concentrated to approximately 40% of its initial volume to provide organic layer, S2-M4.

20. The process of claim 19 wherein the organic layer S2-M4 is heated to 60° C. and to it is added one volume of heptane, then the resulting mixture is cooled to 10° C., stirred for 3 hours and the resulting solid is collected by filtration, washed with 1:1 isopropyl acetate/heptane and dried to provide (1R,2S,5S)-3-((S)-3,3-dimethyl-2-(2,2,2-trifluoroacetamido)butanoyl)-6,6-dimethyl-3-azabicyclo [3.1.0]hexane-2-carboxylic acid, IV.

21. A process for preparing (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1, compound I 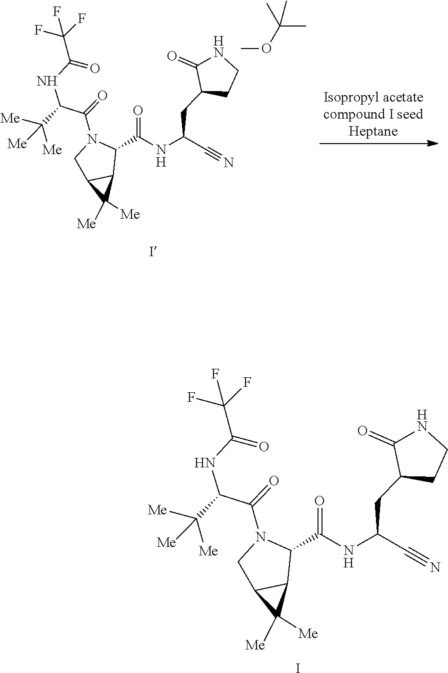
the process comprising the steps (a) to (d):
(a) dissolving (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, methyl tert-butyl ether, compound I′, in isopropyl acetate wherein the concentration of compound I′ in isopropyl acetate is about 7 mL to about 9 mL of isopropyl acetate/1 gram of compound I′;
(b) seeding the solution with 0.5 weight % to 0.75 weight % of (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1
(c) adding heptane to the mixture from step (b) over a period of 6 to 15 hours wherein the amount of heptane added is about 10 mL to about 14 mL heptane/gram of compound I′; and
(d) isolating the resulting (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1.

22. The process of claim 21 wherein the amount of isopropyl acetate used in step (a) is about 8 mL of isopropyl acetate/gram of compound I′.

23. The process of claim 22 wherein the amount of (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1 used to seed the solution in step (b) is about 0.75 w %.

24. The process of claim 23 wherein the amount of heptane added in step (c) is about 12 mL/gram of compound I′.

25. The process of claim 24 wherein the heptane is added over a period of about 10 hours.

26. The process of claim 25 wherein the (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1 is isolated by filtration.

27. The process of claim 26 wherein the (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1 isolated in step (d) has a particle size distribution with a D[v, 0.5] count of about 12 microns to about 18 microns.

28. The process of claim 27 wherein the (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1 isolated in step (d) has a particle size distribution with a D[v, 0.5] count of about 14 microns to about 16 microns.

29. The process of claim 28 wherein the (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, Form 1 isolated in step (d) has a particle size distribution with a D[v, 0.5] count of about 15 microns.

30. The compound (1R,2S,5S)-N-{(1S)-1-cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide, isopropyl acetate solvate.

31. The compound of claim 30 wherein the compound is crystalline.

32. The compound of claim 31 which is characterized by the PXRD pattern [symbol]
